(19) United States
(12) Patent Application Publication
(10) Pub. No.: US 2006/0063772 A1
(43) Pub. Date: Mar. 23, 2006

(54) New compounds

(76) Inventors: Jalaj Arora, Milton (CA); Louise Edwards, Mississauga (CA); Methvin Isaac, Etobicoke (CA); Donald A. McLeod, Salt Lake City, UT (US); Abdelmalik Slassi, Mississauga (CA); Tomislav Stefanac, Burlington (CA); Thomas M. Stormann, Salt Lake City, UT (US); David Wensbo, Sodertalje (SE); Tao Xin, Woodbridge (CA); Annika Kers, Sodertalje (SE); Johan Malmberg, Sodertalje (SE); Karin Oscarsson, Sodertalje (SE)

Correspondence Address: BIRCH, STEWART, KOLASCH & BIRCH, LLP, P.O. BOX 747, 8110 GATEHOUSE ROAD, SUITE 500 EAST, FALLS CHURCH, VA, 22040-0747, US

(21) Appl. No.: 11/270,617

(22) Filed: Nov. 10, 2005

Related U.S. Application Data
(63) Continuation of application No. 10/636,965, filed on Aug. 8, 2003.
(60) Provisional application No. 60/402,039, filed on Aug. 9, 2002.

(51) Int. Cl. A61K 31/496; C07D 403/14

(52) U.S. Cl. 514/253.1; 544/358; 514/254.02; 514/254.05

(57) ABSTRACT

The present invention relates to new compounds of formula I, 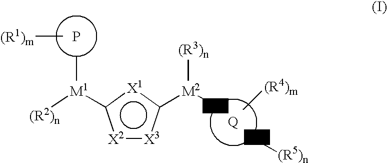
wherein P, Q, X¹, X², X³, X⁴, X⁴, R, R¹, R², R³, R⁴, R⁵, R⁶, R⁷, m, n, o, p and q are defined as in any one of claims 1 to 12, a process for their preparation and new intermediates prepared therein, pharmaceutical formulations containing said compounds and to the use of said compounds in therapy.

### CROSS-REFERENCE TO RELATED APPLICATIONS

The present application is a 37 C.F.R. § 1.53(b) continuation of U.S. application Ser. No. 10/636,965, filed on Aug. 8, 2003, which claims priority on U.S. Provisional Application No. 60/402,039 filed Aug. 9, 2002. The contents of each of the above-identified applications is hereby incorporated by reference.

### FIELD OF THE INVENTION

The present invention relates to a new class of compounds, to pharmaceutical formulations containing said compounds and to the use of said compounds in therapy. The present invention further relates to the process for the preparation of said compounds and to new intermediates prepared therein.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluR) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Eight distinct mGluR subtypes, termed mGluR1 through mGluR8, have been identified by molecular cloning. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluR5, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

Attempts at elucidating the physiological roles of Group I mGluRs suggest that activation of these receptors elicits neuronal excitation. Various studies have demonstrated that Group I mGluRs agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1(1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated. Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders. Because of their physiological and pathophysiological significance, there is a need for new potent mGluR agonists and antagonists that display a high selectivity for mGluR subtypes, particularly the Group I receptor subtype, most particularly the mGluR5 subtype.

The object of the present invention is to provide compounds exhibiting an activity at metabotropic glutamate receptors (mGluRs), especially at the mGluR5 receptor.

### SUMMARY OF THE INVENTION

In one aspect of the invention there are provided compounds having the formula I 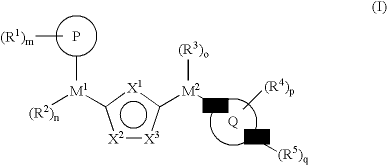
wherein:
P is selected from the group consisting of C₃₋₇alkyl and a 3- to 8-membered ring containing one or more atoms independently selected from C, N, O or S, wherein said ring may be fused with a 5- or 6-membered ring containing one or more atoms independently selected from C, N, O or S;
R¹ is selected from the group consisting of hydrogen, hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, (CO)R⁶, O(CO)R⁶, O(CO)OR⁶, C₁₋₆alkylOR⁶, OC₂₋₆alkylOR⁶, C₁₋₆alkyl(CO)R⁶, OC₁₋₆alkyl(CO)R⁶, C₀₋₆alkylCO₂R⁶, OC₁₋₆alkylCO₂R⁶, C₀₋₆alkylcyano, OC₂₋₆alkylcyano, C₀₋₆alkylNR⁶R⁷, OC₂₋₆alkylNR⁶R⁷, C₁₋₆alkyl(CO)NR⁶R⁷, OC₁₋₆alkyl(CO)NR⁶R⁷, C₀₋₆alkylNR⁶(CO)R⁷, OC₂₋₆alkylNR⁶(CO)R⁷, C₀₋₆alkylNR⁶(CO)NR⁶R⁷, C₀₋₆alkylSR⁶, OC₂₋₆alkylSR⁶, C₀₋₆alkyl(SO)R⁶, OC₂₋₆alkyl(SO)R⁶, C₀₋₆alkylSO₂R⁶, OC₂₋₆alkylSO₂R⁶, CO₀₋₆alkyl(SO₂)NR⁶R⁷, OC₂₋₆alkyl(SO₂)NR⁶R⁷, C₀₋₆alkylNR⁶(SO₂)R⁷, OC₂₋₆alkylNR⁶(SO₂)R⁷, C₀₋₆alkylNR⁶(SO₂)NR⁶R⁷, OC₂₋₆alkylNR⁶(SO₂)NR⁶R⁷, (CO)NR⁶R⁷, O(CO)NR⁶R⁷, NR⁶OR⁷, C₀₋₆alkylNR⁶(CO)OR⁷, OC₂₋₆alkylNR⁶(CO)OR⁷, SO₃R⁶ and a 5- or 6-membered ring containing one or more atoms independently selected from C, N, O or S, wherein said ring may be substituted by one or more A;
M¹ is selected from the group consisting of a bond, C₁₋₃alkyl, C₂₋₃alkenyl, C₂₋₃alkynyl, C₀₋₄alkyl(CO)C₀₋₄alkyl, C₀₋₃alkylOC₀₋₃alkyl, C₀₋₃alkyl(CO)NR⁷R⁶, C₀₋₃alkyl(CO)NR⁷R⁶C₁₋₃alkyl, C₀₋₄alkylNR⁷R⁶, C₀₋₃alkylSC₀₋₃alkyl, C₀₋₃alkyl(SO)C₀₋₃alkyl and C₀₋₃alkyl(SO₂)C₀₋₃alkyl;
X¹, X² and X³ are independently selected from the group consisting of CR, CO, N, NR, O and S;
R is selected from the group consisting of hydrogen, C₀₋₃alkyl, halo, C₀₋₃alkylOR⁵, C₀₋₃alkylNR⁵R⁶, C₀₋₃alkyl(CO)OR⁵, C₀₋₃alkylNR⁵R⁶ and C₀₋₃alkylaryl;
R² is selected from the group consisting of hydrogen, hydroxy, oxo, ═NR⁶, ═NOR⁶, C₁₋₄alkylhalo, halo, C₁₋₄alkyl, OC₁₋₄alkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₀₋₄alkyl, C₀₋₄alkylcyano, C₁₋₄alkylOR⁶ and C₀₋₄alkylNR⁶R⁷;
M² is selected from the group consisting of a bond, C₁₋₃alkyl, C₂₋₃alkenyl, C₂₋₃alkynyl, C₀₋₄alkyl(CO)C₀₋₄alkyl, C₀₋₃alkylOC₀₋₃alkyl, C₀₋₃alkylNR⁶C₁₋₃alkyl, C₀₋₃alkyl(CO)NR⁶, C₀₋₄alkylNR⁶R⁷, C₀₋₃alkylSC₀₋₃alkyl, C₀₋₃alkyl(SO)C₀₋₃alkyl and C₀₋₃alkyl(SO₂)C₀₋₃alkyl;
R³ is selected from the group consisting of hydrogen, hydroxy, oxo, ═NR⁶, ═NOR⁶, C₁₋₄alkylhalo, halo, C₁₋₄alkyl, OC₁₋₄alkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₀₋₄alkylcyano, C₁₋₄alkylOR⁶ and C₀₋₄alkylNR⁶R⁷;
X⁴ is selected from C, CR or N;
X⁵ is selected from C, CR or N;
Q is a 4- to 8-membered ring or bicycle containing one or more atoms independently selected from C, N, O or S, wherein said ring or bicycle may be fused with a 5- or 6-membered ring containing one or more atoms independently selected from C, N, O or S and wherein the fused ring may be substituted by one or more A;
R⁴ is selected from the group consisting of hydrogen, hydroxy, halo, nitro, oxo, C₁₋₆alkylhalo, C₁₋₆alkyl, OC₁₋₆alkyl, C₀₋₆alkylC₃₋₆cycloalkyl, C₀₋₆alkylaryl, OC₀₋₆alkylaryl, (CO)R⁶, O(CO)R⁶, C₁₋₆alkylOR⁶, OC₂₋₆alkylOR⁶, C₁₋₆alkyl(CO)R⁶, OC₁₋₆alkyl(CO)R⁶, C₀₋₆alkylCO₂R⁶, OC₁₋₆alkylCO₂R⁶, C₀₋₆alkylcyano, OC₁₋₆alkylcyano, C₀₋₆alkylNR⁶R⁷, OC₂₋₆alkylNR⁶R⁷, C₀₋₆alkyl(CO)NR⁶R⁷, OC₀₋₆alkyl(CO)NR⁶R⁷, C₀₋₆alkylNR⁶(CO)R⁷, OC₂₋₆alkylNR⁶(CO)R⁷, C₀₋₆alkylNR⁶(CO)NR⁶R⁷, C₀₋₆alkylSR⁶, OC₂₋₆alkylSR⁶, C₀₋₆alkyl(SO)R⁶, OC₂₋₆alkyl(SO)R⁶, C₀₋₆alkylSO₂R⁶, OC₀₋₆alkylSO₂R⁶, C₀₋₆alkyl(SO₂)NR⁶R⁷, OC₀₋₆alkyl(SO₂)NR⁶R⁷, C₀₋₆alkylNR⁶(SO₂)R⁷, OC₂₋₆alkylNR⁶(SO₂)R⁷, NR⁶OR⁷, NR⁶(CO)OR⁷, SO₃R⁶ and a 5- or 6-membered ring containing one or more atoms independently selected from C, N, O or S, wherein said ring may be substituted by one or more A;
R⁵ is selected from the group consisting of hydrogen, hydroxy, halo, oxo, C₁₋₆alkylhalo, OC₁₋₆alkylhalo, C₁₋₆alkyl, OC₁₋₆alkyl, C₀₋₆alkylC₃₋₆cycloalkyl, C₀₋₆alkylaryl, OC₀₋₆alkylaryl, (CO)R⁶, O(CO)R⁶, O(CO)OR⁶, (CO)OR⁶, C₁₋₆alkylOR⁶, OC₂₋₆alkylOR⁶, C₁₋₆alkyl(CO)R⁶, OC₁₋₆alkyl(CO)R⁶, C₀₋₆alkylCO₂R⁶, OC₁₋₆alkylCO₂R⁶, C₀₋₆alkylcyano, OC₀₋₆alkylcyano, C₀₋₆alkylNR⁶R⁷, OC₂₋₆alkylNR⁶R⁷, C₁₋₆alkyl(CO)NR⁶R⁷, C₀₋₆alkyl(CO)heteroaryl, C₀₋₆alkyl(CO)aryl, OC₁₋₆alkyl(CO)NR⁶R⁷, C₁₋₆alkyl(CO)NR⁶R⁷, C₀₋₆alkylNR⁶(CO)R⁷, OC₂₋₆alkylNR⁶(CO)R⁷, C₀₋₆alkylNR⁶(CO)NR⁶R⁷, C₁₋₆alkylNR⁶(CO)OR⁷ C₀₋₆alkylSR⁶, OC₂₋₆alkylSR⁶, C₀₋₆alkyl(SO)R⁶, OC₁₋₆alkyl(SO)R⁶, C₀₋₆alkylSO₂R⁶, OC₀₋₆alkylSO₂R⁶, C₀₋₆alkyl(SO₂)NR⁶R⁷, OC₀₋₆alkyl(SO₂)NR⁶R⁷,C₀₋₆alkylNR⁶(SO₂)R⁷, OC₂₋₆alkylNR⁶(SO₂)R⁷, C₀₋₆alkylNR⁶(SO₂)NR⁶R⁷, OC₂₋₆alkylNR⁶(SO₂)NR⁶R⁷, (CO)NR⁶R⁷, O(CO)NR⁶R⁷, NR⁶OR⁷, NR⁶(CO)OR⁷, SO₃R⁶ and a 5-or 6-membered ring containing one or more atoms independently selected from C, N, O or S, wherein said ring may be substituted by one or more A;
R⁶ and R⁷ are independently selected from hydrogen, C₁₋₆alkyl, C₀₋₆alkylC₃₋₆cycloalkyl, C₀₋₆alkylaryl, C₁₋₆alkylheteroaryl and a 5- or 6-membered ring containing one or more atoms independently selected from C, N, O or S, and wherein R⁶ and R⁷ may together form a 5- or 6-membered ring containing one or more atoms independently selected from C, N, O or S;
wherein any C₁₋₆alkyl, C₂₋₆alkenyl, C₂₋₆alkynyl, C₀₋₆alkylC₃₋₆cycloalkyl, CO₀₋₆alkylaryl and C₀₋₆alkylheteroaryl defined under R¹, R², R³, R⁴, R⁵, R⁶ and R⁷ may be substituted by one or more A;
A is selected from the group consisting of hydrogen, hydroxy, oxo, halo, nitro, C₁₋₆alkylhalo, OC₁₋₆alkylhalo, C₁₋₆alkyl, C₀₋₄alkylC₃₋₆cycloalkyl, C₂₋₆alkenyl, OC₁₋₆alkyl, C₀₋₃alkylaryl, C₁₋₆alkylOR⁶, OC₂₋₆alkylOR⁶, C₁₋₆alkylSR⁶, OC₂₋₆alkylSR⁶, (CO)R⁶, O(CO)R⁶, OC₂₋₆alkylcyano, C₀₋₆alkylcyano, C₀₋₆alkylCO₂R⁶, OC₁₋₆alkylCO₂R⁶, O(CO)OR⁶, OC₁₋₆alkyl(CO)R⁶, C₁₋₆alkyl(CO)R⁶, NR⁶OR⁷, C₀₋₆alkylNR⁶R⁷, OC₂₋₆alkylNR⁶R⁷, C₀₋₆alkyl(CO)NR⁶R⁷, OC₁₋₆alkyl(CO)NR⁶R⁷, OC₂₋₆alkylNR⁶(CO)R⁷, C₀₋₆alkylNR⁶(CO)R⁷, C₀₋₆alkylNR⁶(CO)NR⁶R⁷, O(CO)NR⁶R⁷, NR⁶(CO)OR⁷, C₀₋₆alkyl(SO₂)NR⁶R⁷, OC₂₋₆alkyl(SO₂)NR⁶R⁷, C₀₋₆alkylNR⁶(SO₂)R⁷, OC₂₋₆alkylNR⁶(SO₂)R⁷, SO₃R⁶, C₁₋₆alkylNR⁶(SO₂)NR⁶R⁷, OC₂₋₆alkyl(SO₂)R⁶, C₀₋₆alkyl(SO₂)R⁶, C₀₋₆alkyl(SO)R⁶ and OC₂₋₆alkyl(SO)R⁶;
m and p are independently selected from the group consisting of 0, 1, 2, 3 and 4;
n, o and q are each independently selected from 0, 1, 2 or 3;
or salt thereof.

In another aspect of the invention there are provided compounds according to claim 1 wherein:
P is selected from the group consisting of a 3- to 8-membered ring containing one or more atoms independently selected from C, N, O or S, wherein said ring may be fused with a 5- or 6-membered ring containing one or more atoms independently selected from C, N, O or S;
M¹ is a bond;
M² is selected from the group consisting of a bond, C₁alkyl, CO,
X⁴ is N;
X⁵ is N;
Q is a 6-membered ring or bicycle containing two N atoms, wherein said ring or bicycle may be fused with a 5- or 6-membered ring containing one or more atoms independently selected from C, N, O or S and wherein the fused ring may be substituted by one or more A;
R⁵ is selected from the group consisting of (CO)OR⁶ and (CS)OR⁶, (CO)SR⁶, CONR6R7 wherein, R⁶ are independently selected from the group consisting of methyl and ethyl, propyl, ipropyl, n-butyl and i-butyl;
m is selected from 1 and 2;
n is 0;
o is selected from 0, and 1;
p is selected from 0, 1 and 2; and
q is selected from 0 and 1;or salt thereof
with the proviso that the compound is not:
1-Piperazinecarboxylic acid, 4-[5-(4-chlorophenyl)-4-(4-pyridinyl)-1H-pyrazol-3-yl]-methyl ester,
1-Piperazinecarboxylic acid, 4-[5-phenyl-4-(4-pyridinyl)-1H-pyrazol-3-yl]-ethyl ester,
1-Piperazinecarboxylic acid-4-[[4-(10Hphenothiazine-2-yl)-2-thiazolyl]methyl]-methyl ester,
1-piperazinecarboxylic acid, 4-[[4-[3,5-bis(1,1-dimethylethyl)-4-hydroxyphenyl]-2-thizolyl]methyl]-methyl ester monohydrochloride,
1-piperazinecarboxylic acid, 4-[[4-[3,5-bis(1,1-dimethylethyl)-4-hydroxyphenyl]-2-thizolyl]methyl]-methyl ester,
1-Piperazinecarboxylic acid, 4-[[5-[4-(trifluoromethyl)-3-pyridinyl]-1,2,4-oxadiazol-3-yl]carbonyl]-ethyl ester,
1-Piperazinecarboxylic acid, 4-[1-(acetylamino)-4-(4-bromophenyl)-1H-imidazol-2-yl]-ethyl ester,
1-Piperazinecarboxylic acid, 4-[[2-(3-pyridinyl)-4-thiazolidinyl]carbonyl]-ethyl ester,
1-Piperazinecarboxylic acid, 4-[[2-(3-pyridinyl)-4-thiazolidinyl]carbonyl]-ethyl ester dihydrochloride,
1-Piperazinecarboxylic acid, 4-[5-(1-methyl-5-nitro-1H-imidazol-2-yl)-1,3,4-thiadiazol-2-yl]-ethyl ester, and
1-Piperazinecarboxylic acid, 4(4,5-diphenyl-2-oxazolyl)-ethyl ester.

In a further aspect of the invention there are provided compounds of formula 1 wherein:
P is phenyl;
M¹ is a bond;
M² is selected from the group consisting of a bond, C₁alkyl
q is 1, m is 1, n is 0, o is;
X1 is selected from N and C, X2 is O and X3 is N;
X⁴ is N;
X⁵ is N;
Q is a 6-membered ring; and
R⁵ is (CO)OR⁸ wherein R⁸ is selected from methyl and ethyl

Specific embodiments of the invention include:
4-(5-m-Tolyl-[1,2,4]oxadiazol-3-ylmethyl)-piperazine-1-carboxylic acid ethyl ester hydrochloride,
4-[5-(3-Methoxyphenyl)-[1,2,4]oxadiazol-3-ylmethyl)-piperazine-1-carboxylic acid ethyl ester hydrochloride,
4-[5-(3-Trifluoromethyl-phenyl)-[1,2,4]oxadiazol-3-ylmethyl]-piperazine-1-carboxylic acid ethyl ester,
4-[5-(3-Cyano-phenyl)-[1,2,4]oxadiazol-3-ylmethyl]-piperazine-1-carboxylic acid ethyl ester),
4-[5-(3-Fluoro-phenyl)-[1,2,4]oxadiazol-3-ylmethyl]-piperazine-1-carboxylic acid ethyl ester,
4-[5-(3-Iodo-phenyl)-[1,2,4]oxadiazol-3-ylmethyl]-piperazine-1-carboxylic acid ethyl ester,
4-[5-(3-Chloro-phenyl)-[1,2,4]oxadiazol-3-ylmethyl]-piperazine-1-carboxylic acid ethyl ester,
4-[5-(3-Trifluoromethoxy-phenyl)-[1,2,4]oxadiazol-3-ylmethyl]-piperazine-1-carboxylic acid ethyl ester,
4-[5-(3-Bromo-phenyl)-[1,2,4]oxadiazol-3-ylmethyl]-piperazine-1-carboxylic acid ethyl ester,
4-(5-m-Tolyl-[1,2,4]oxadiazol-3-ylmethyl)-piperazine-1-carboxylic acid methyl ester,
4-(5-m-Tolyl-[1,2,4]oxadiazol-3-ylmethyl)-piperazine-1-carboxylic acid propyl ester,
4-(5-m-Tolyl-[1,2,4]oxadiazol-3-ylmethyl)-piperazine-1-carboxylic acid butyl ester,
4-[5-(3-Methoxy-phenyl)-[1,2,4]oxadiazol-3-ylmethyl]-2-methyl-piperazine-1-carboxylic acid ethyl ester,
4-(5-m-Tolyl-[1,2,4]oxadiazol-3-ylmethyl)-piperazine-1-carboxylic acid isopropyl ester,
4-[1-(5-(3-Methyl-phenyl)-[1,2,4]oxadiazol-3-yl)-ethyl]-piperazine-carboxylic acid ethyl ester or
4-[5-(3-Furan-3-yl-phenyl)-[1,2,4]oxadiazol-3-ylmethyl]-piperazine-1-carboxylic acid ethyl ester,
4-{Cyano-[5-(2-fluoro-5-methyl-phenyl)-isoxazol-3-yl]-methyl}-piperazine-1-carboxylic acid ethyl ester,
4-[5-(3-Chloro-phenyl)-[1,2,4]oxadiazol-3-ylmethyl]-2-oxo-piperazine-1-carboxylic acid ethyl ester,
4-[1-(5-m-Tolyl-[1,2,4]oxadiazol-3-yl)-ethyl]-piperazine-1-carboxylic acid ethyl-methyl-amide,
(R)-and (S)-4-[1-(5-(3-Methyl-phenyl)-[1,2,4]oxadiazol-3-yl)-ethyl]-piperazine-carboxylic acid ethyl ester,
(R)-and (S)-4-[1-(5-(3-Methyl-phenyl)-[1,2,4]oxadiazol-3-yl)-ethyl]-piperazine-carboxylic acid ethyl ester,
4-{1-[5-(3-Chloro-phenyl)-[1,2,4]oxadiazol-3-yl]-propyl}-piperazine-1-carboxylic acid ethyl ester,
(S)-4-{1-[5-(5-Chloro-2-fluoro-phenyl)-[1,2,4]oxadiazol-3-yl]-ethyl}-piperazine-1-carboxylic acid ethyl ester,
(S)-{1-[5-(2-Fluoro-5-methyl-phenyl)-[1,2,4]oxadiazol-3-yl]-ethyl}-piperazine-1-carboxylic acid ethyl ester,
(S)-4-{1-[5-(3-Chloro-phenyl)-[1,2,4]oxadiazol-3-yl]-ethyl}-piperazine-1-carboxylic acid ethyl ester,
(R)-4-[5-(2-Fluoro-5-methyl-phenyl)-[1,2,4]oxadiazol-3-ylmethyl]-2-methyl-piperazine-1-carboxylic acid ethyl ester,
(S)-4-[5-(2-Fluoro-5-methyl-phenyl)-[1,2,4]oxadiazol-3-ylmethyl]-2-methyl-piperazine-1-carboxylic acid ethyl ester,
(R)-3-Methyl-4-(5-m-tolyl-[1,2,4]oxadiazol-3-ylmethyl)-piperazine-1-carboxylic acid ethyl ester,
(S)-3-Methyl-4-(5-m-tolyl-[1,2,4]oxadiazol-3-ylmethyl)-piperazine-1-carboxylic acid ethyl ester, 4-[5-(3-Methylsulfanyl-phenyl)-[1,2,4]oxadiazol-3-ylmethyl]-piperazine-1-carboxylic acid ethyl ester,
4-[5-(2-Fluoro-5-methyl-phenyl)-[1,2,4]oxadiazol-3-ylmethyl]-piperazine-1-carboxylic acid ethyl ester,
4-[5-(3-Chloro-phenyl)-isoxazol-3-ylmethyl]-piperazine-1-carboxylic acid ethyl ester,
4-[5-(2-Fluoro-5-methyl-phenyl)-[1,2,4]oxadiazol-3-yl-(R)-methyl]-3-methyl-piperazine-1-carboxylic acid ethyl ester,
4-[5-(2-Fluoro-5-methyl-phenyl)-[1,2,4]oxadiazol-3-yl-(S)-methyl]-3-methyl-piperazine-1-carboxylic acid ethyl ester,
4-[5-(5-Bromo-2-fluoro-phenyl)-[1,2,4]oxadiazol-3-ylmethyl]-piperazine-1-carboxylic acid ethyl ester,
4-[5-(2,5-Dichloro-phenyl)-[1,2,4]oxadiazol-3-ylmethyl]-piperazine-1-carboxylic acid ethyl ester,
4-(5-Thiophen-3-yl-isoxazol-3-ylmethyl)-piperazine-1-carboxylic acid ethyl ester,
4-[5-(2-Fluoro-5-methyl-phenyl)-isoxazol-3-ylmethyl]-piperazine-1-carboxylic acid ethyl ester,
4-{1-[5-(3-Chloro-phenyl)-isoxazol-3-yl]-ethyl}-piperazine-1-carboxylic acid ethyl ester,
4-{1-[5-(2-Fluoro-5-methyl-phenyl)-isoxazol-3-yl]-ethyl}-piperazine-1-carboxylic acid ethyl ester,
(R)- and (S)-4-{1-[5-(2-Fluoro-5-methyl-phenyl)-isoxazol-3-yl]-ethyl}-piperazine-1-carboxylic acid ethyl ester enantiomers,
4-{1-[5-(2-Fluoro-5-methyl-phenyl)-isoxazol-3-yl]-propyl}-piperazine-1-carboxylic acid ethyl ester,
4-{Cyclopropyl-[5-(2-fluoro-5-methyl-phenyl)-isoxazol-3-yl]-methyl}-piperazine-1-carboxylic acid ethyl ester,
4-{1-[5-(2-Fluoro-5-methyl-phenyl)-isoxazol-3-yl]-ethyl}-3-(R)-methyl-piperazine-1-carboxylic acid ethyl ester, (2 diastereomers)
4-{1-[5-(2-Fluoro-5-methyl-phenyl)-isoxazol-3-yl]-ethyl}-3-(S)-methyl-piperazine-1-carboxylic acid ethyl ester, (2 diastereomers)
4-{1-[5-(3-Chloro-phenyl)-isoxazol-3-yl]-ethyl}-3-(R)-methyl-piperazine-1-carboxylic acid ethyl ester, (2 diastereomers)
4-{1-[5-(3-Chloro-phenyl)-isoxazol-3-yl]-ethyl}-3-(S)-methyl-piperazine-1-carboxylic acid ethyl ester, (2 diastereomers)
4-{1-[5-(3-Chloro-phenyl)-isoxazol-3-yl]-ethyl}-2-(R)-methyl-piperazine-1-carboxylic acid ethyl ester, (2 diastereomers)
4-{1-[5-(3-Chloro-phenyl)-isoxazol-3-yl]-ethyl}-2-(S)-methyl-piperazine-1-carboxylic acid ethyl ester, (2 diastereomers)
(R)-4-[5-(3-Chloro-phenyl)-isoxazol-3-ylmethyl]-3-methyl-piperazine-1-carboxylic acid ethyl ester,
(R)-4-[5-(2-Fluoro-5-methyl-phenyl)-isoxazol-3-ylmethyl]-3-methyl-piperazine-1-carboxylic acid ethyl ester,
(S)-4-[5-(3-Chloro-phenyl)-isoxazol-3-ylmethyl]-3-methyl-piperazine-1-carboxylic acid ethyl ester,
(S)-4-[5-(2-Fluoro-5-methyl-phenyl)-isoxazol-3-ylmethyl]-3-methyl-piperazine-1-carboxylic acid ethyl ester,
4-[5-(3-Chloro-phenyl)-oxazol-2-ylmethyl]-piperazine-1-carboxylic acid ethyl ester,
4-[5-(5-Chloro-2-fluoro-phenyl)-[1,2,4]oxadiazol-3-ylmethyl]-piperazine-1-carboxylic acid ethyl ester,
4-[5-(2-Chloro-5-methyl-phenyl)-[1,2,4]oxadiazol-3-ylmethyl]-piperazine-1-carboxylic acid ethyl ester,
4-{1-[5-(3-Chloro-phenyl)-[1,2,4]oxadiazol-3-yl]-ethyl}-piperazine-1-carboxylic acid ethyl ester,
4-{1-[5-(3-Chloro-phenyl)-[1,2,4]oxadiazol-3-yl]-ethyl}-3-(S)-methyl-piperazine-1-carboxylic acid ethyl ester,
4-{1-[5-(3-Chloro-phenyl)-[1,2,4]oxadiazol-3-yl]-ethyl}-3-(R)-methyl-piperazine-1-carboxylic acid ethyl ester,
4-{1-[5-(3-Chloro-phenyl)-[1,2,4]oxadiazol-3-yl]-ethyl}-3-(R)-methyl-piperazine-1-carboxylic acid ethyl ester,
4-[5-(5-Chloro-2-fluoro-phenyl)-[1,3,4]oxadiazol-2-ylmethyl]-piperazine-1-carboxylic acid ethyl ester,
4-{1-[5-(5-Chloro-2-fluoro-phenyl)-[1,3,4]oxadiazol-2-yl]-ethyl}-piperazine-1-carboxylic acid ethyl ester,
4-[5-(2-Fluoro-5-methyl-phenyl)-[1,3,4]oxadiazol-2-ylmethyl]-piperazine-1-carboxylic acid ethyl ester,
4-{1-[5-(2-Fluoro-5-methyl-phenyl)-[1,3,4]oxadiazol-2-yl]-ethyl}-piperazine-1-carboxylic acid ethyl ester,
4-(5-m-Tolyl-isoxazol-3-ylmethyl)-piperazine-1-carboxylic acid ethyl ester,
4-[5-(3-methoxy-phenyl)-isoxazol-3-ylmethyl]-piperazine-1-carboxylic acid ethyl ester,
4-[5-(3-cyano-phenyl)-isoxazol-3-ylmethyl]-piperazine-1-carboxylic acid ethyl ester,
4-[5-(3-Formyl-phenyl)-isoxazol-3-ylmethyl]-piperazine-1-carboxylic acid ethyl ester,
4-[5-(5-Cyano-2-fluoro-phenyl)-isoxazol-3-ylmethyl]-piperazine-1-carboxylic acid ethyl ester,
4-[5-(5-Chloro-2-fluoro-phenyl)-isoxazol-3-ylmethyl]-piperazine-1-carboxylic acid ethyl ester,
4-{1-[5-(5-Chloro-2-fluoro-phenyl)-isoxazol-3-yl]-ethyl}-piperazine-1-carboxylic acid ethyl ester,
4-[1-(5-m-Tolyl-isoxazol-3-yl)-ethyl]-piperazine-1-carboxylic acid ethyl ester,
4-{1-[5-(3-Methoxy-phenyl)-isoxazol-3-yl]-ethyl}-piperazine-1-carboxylic acid ethyl ester,
4-{1-[5-(3-Cyano-phenyl)-isoxazol-3-yl]-ethyl}-piperazine-1-carboxylic acid ethyl ester,
4-{1-[5-(5-Cyano-2-fluoro-phenyl)-isoxazol-3-yl]-ethyl}-piperazine-1-carboxylic acid ethyl ester,
4-{1-[5-(2-Methyl-pyridin-4-yl)-isoxazol-3-yl]-ethyl}piperazine-1-carboxylic acid ethyl ester,
4-{1-[5-(5-Chloro-2-fluoro-phenyl)-isoxazol-3-yl]-2,2,2-trifluoro-ethyl}-piperazine-1-carboxylic acid ethyl ester,
4-[5-(2-Fluoro-5-iodo-phenyl)-[1,2,4]oxadiazol-3-ylmethyl]-piperazine-1-carboxylic acid ethyl ester,
4-[5-(2-Hydroxy-5-methyl-phenyl)-[1,2,4]oxadiazol-3-ylmethyl]-piperazine-1-carboxylic acid ethyl ester,
4-[5-(5-Chloro-2-hydroxy-phenyl)-[1,2,4]oxadiazol-3-ylmethyl]-piperazine-1-carboxylic acid ethyl ester, or
a salt thereof

In a further aspect of the invention there is provided pharmaceutical formulations comprising a therapeutically effective amount of a compound of formula I and a pharmaceutically acceptable diluent, excipients and/or inert carrier.

In yet a further aspect of the invention there is provided a pharmaceutical formulation including a compound of formula I for the treatment of mGluR5 receptor-mediated disorders, and particularly neurological disorders, psychiatric disorders, acute and chronic pain.

In still a further aspect of the invention there is provided a compound of formula I for use in therapy for the treatment of mGluR5 receptor-mediated disorders, and particularly neurological disorders, psychiatric disorders, acute and chronic pain.

In another aspect of the invention there is provided a process for the preparation of a compound of formula I, and the intermediates provided therein.

These and other aspects of the present invention are described in greater detail herein below.

### DETAILED DESCRIPTION OF THE INVENTION

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl. The term “C₁₋₃alkyl” refers to an alkyl group having 1 to 3 carbon atoms, and may be methyl, ethyl, n-propyl and i-propyl.

In this specification, unless stated otherwise, the term “cycloalkyl” refers to an optionally substituted, saturated cyclic hydrocarbon ring system. The term “C₃₋₇cycloalkyl” may be cyclopropyl, cyclobutyl, cyclopentyl, cyclohexyl and cycloheptyl.

In this specification, unless stated otherwise, the term “alkenyl” includes both straight and branched chain alkenyl groups. The term “C₂₋₆alkenyl” refers to an alkenyl group having 2 to 6 carbon atoms and one or two double bonds, and may be, but is not limited to vinyl, allyl, propenyl, i-propenyl, butenyl, i-butenyl, crotyl, pentenyl, i-pentenyl and hexenyl.

In this specification, unless stated otherwise, the term “alkynyl” includes both straight and branched chain alkynyl groups. The term “C₂₋₆alkynyl” refers to a group having 2 to 6 carbon atoms and one or two triple bonds, and may be, but is not limited to ethynyl, propargyl, butynyl, i-butynyl, pentynyl, i-pentynyl and hexynyl.

The term “aryl” refers to an optionally substituted monocyclic or bicyclic hydrocarbon ring system containing at least one unsaturated aromatic ring. Examples and suitable values of the term “aryl” are phenyl, naphthyl, 1,2,3,4-tetrahydronaphthyl, indyl and indenyl.

In this specification, unless stated otherwise, the term “heteroaryl” refers to an optionally substituted, unsaturated cyclic or bicyclic hydrocarbon ring system comprising at least one heteroatom and includes, but is not limited to furyl, isoxazolyl, isothiazolyl, oxazolyl, thiazolyl, pyrazinyl, pyridazinyl, pyridyl, pyrimidyl, pyrrolyl, thiazolyl, imidazolyl, imidazolinyl, pyrazolinyl, tetrahydropyranyl, indolinyl, indolyl, chromanyl, osichromanyl, quinolinyl, benzothiazolyl, quinoxalinyl, azulenyl, indenyl, benzimidazolyl, indazolyl, benzofuranyl and dihydro-benzo-oxazin-one.

In this specification, unless stated otherwise, the term “5- or 6-membered ring containing one or more atoms independently selected from C, N, O or S” includes aromatic and heteroaromatic rings as well as carbocyclic and heterocyclic rings which may be saturated or unsaturated. Examples of such rings may be, but are not limited to furyl, isoxazolyl, isothiazolyl, oxazolyl, pyrazinyl, pyrazolyl, pyridazinyl, pyridyl, pyrimidyl, pyrrolyl, thiazolyl, thienyl, imidazolyl, imidazolidinyl, imidazolinyl, triazolyl, morpholinyl, piperazinyl, piperidyl, piperidonyl, pyrazolidinyl, pyrazolinyl, pyrrolidinyl, pyrrolinyl, tetrahydropyranyl, thiomorpholinyl, phenyl, cyclohexyl, cyclopentyl and cyclohexenyl.

In this specification, unless stated otherwise, the terms “3- to 8-membered ring containing one or more atoms independently selected from C, N, O or S” includes aromatic and heteroaromatic rings as well as carbocyclic and heterocyclic rings which may be saturated or unsaturated. Examples of such rings may be, but are not limited to imidazolidinyl, imidazolinyl, morpholinyl, piperazinyl, piperidyl, piperidonyl, pyrazolidinyl, pyrazolinyl, pyrrolidinyl, pyrrolinyl, tetrahydropyranyl or thiomorpholinyl, tetrahydrothiopyranyl, furyl, pyrrolyl, isoxazolyl, isothiazolyl, oxazolyl, oxazolidinonyl, pyrazinyl, pyrazolyl, pyridazinyl, pyridyl, pyrimidyl, pyrrolyl, thiazolyl, thienyl, imidazolyl, triazolyl, phenyl, cyclopropyl, aziridinyl, cyclobutyl, azetidinyl, cyclopentyl, cyclopentenyl, cyclohexyl, cyclohexenyl, cycloheptyl, cycloheptenyl, cyclooctyl and cyclooctenyl.

In this specification, unless stated otherwise, the term “3- to 8-membered ring containing one or more atoms independently selected from C, N, O or S, which group may optionally be fused with a 5- or 6-membered ring containing one or more atoms independently selected from C, N, O or S” includes aromatic and heteroaromatic rings as well as carbocyclic and heterocyclic rings which may be saturated or unsaturated. Examples of such rings may be, but are not limited to naphthyl, norcaryl, chromyl, isochromyl, indanyl, benzoimidazol or tetralinyl, benzooxazolyl, benzothiazolyl, benzofuryl, benzothienyl, benzotriazolyl, indolyl, azaindolyl, indazolyl, indolinyl, isoindolinyl, benzimidazolyl, oxadiazolyl, thiadiazolyl, quinolinyl, quinoxalinyl and benzotriazolyl.

In this specification, unless stated otherwise, the term “═NR⁶” and “═NOR⁶” include imino- and oximogroups carrying an R⁶ substituent and may be, or be part of, groups including, but not limited to iminoalkyl, iminohydroxy, iminoalkoxy, amidine, hydroxyamidine and alkoxyamidine.

In the case where a subscript is the integer 0 (zero) the group to which the subscript refers to indicates that the group is absent, i.e. there is a direct bond between the groups.

In this specification, unless stated otherwise, the term “bond” may be a saturated or unsaturated bond.

In this specification, unless stated otherwise, the term “halo” may be fluoro, chloro, bromo or iodo.

In this specification, unless stated otherwise, the term “alkylhalo” means an alkyl group as defined above, which is substituted with one or more halo. The term “C₁₋₆alkylhalo” may include, but is not limited to fluoromethyl, difluoromethyl, trifluoromethyl, fluoroethyl, difluoroethyl, bromopropyl. The term “OC₁₋₆alkylhalo” may include, but is not limited to fluoromethoxy, difluoromethoxy, trifluoromethoxy, fluoroethoxy and difluoroethoxy.

In one embodiment of the invention there is provided compounds of formula I wherein P is C₃₋₇alkyl. In another embodiment P is a 3- to 8 membered ring containing one or more atoms independently selected from C, N, O or S, wherein said ring may be fused with a 5- or 6-membered ring containing one or more atoms independently selected from C, N, O or S.

In a further embodiment P is a 5- or 6 membered ring. In yet a further embodiment P is selected from aromatic and heteroaromatic rings. In still a further embodiment P is phenyl, pyridinyl or thiophenyl.

P is optionally substituted with 1, 2, 3 or 4 groups R¹ wherein the number of R¹ substituents on the P ring is designated by the term m. In suitable embodiments of the invention m is 1 or 2, in further embodiments of the invention m is 1.

In a suitable embodiment of the invention R₁ is selected from the group consisting of hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CO, (CO)R⁶, O(CO)R⁶, O(CO)OR⁶, C₁₋₆alkylOR⁶, OC₂₋₆alkylOR⁶, C₁₋₆alkyl(CO)R⁶, OC₁₋₆alkyl(CO)R⁶, C₀₋₆alkylCO₂R⁶, OC₁₋₆alkylCO₂R⁶, C₀₋₆alkylcyano, OC₂₋₆alkylcyano, C₀₋₆alkylNR⁶R⁷, OC₂₋₆alkylNR⁶R⁷, C₁₋₆alkyl(CO)NR⁶R⁷, OC₁₋₆alkyl(CO)NR⁶R⁷, C₀₋₆alkylNR⁶(CO)R⁷, OC₂₋₆alkylNR⁶(CO)R⁷, C₀₋₆alkylNR⁶(CO)NR⁶R⁷, C₀₋₆alkylSR⁶, OC₂₋₆alkylSR⁶, C₀₋₆alkyl(SO)R⁶, OC₂₋₆alkyl(SO)R⁶, C₀₋₆alkylSO₂R⁶, OC₂₋₆alkylSO₂R⁶, C₀₋₆alkyl(SO₂)NR⁶R⁷, OC₂₋₆alkyl(SO₂)NR⁶R⁷, C₀₋₆alkylNR⁶(SO₂)R⁷, OC₂₋₆alkylNR⁶(SO₂)R⁷, C₀₋₆alkylNR⁶(SO₂)NR⁶R⁷, OC₂₋₆alkylNR⁶(SO₂)NR⁶R⁷, (CO)NR⁶R⁷, O(CO)NR⁶R⁷, NR⁶OR⁷, C₀₋₆alkylNR⁶(CO)OR⁷, OC₂₋₆alkylNR⁶(CO)OR⁷, SO₃R⁶ and a 5- or 6-membered ring containing one or more atoms independently selected from C, N, O or S, wherein said ring may be substituted by one or more A.

More suitably R1 is selected from the group consisting of Meo, OH, CN, furyl, OCF₃,CHO, SMe and CF3.

In another suitable embodiment, P is a 6-member aryl or heteroaryl ring, and R¹ is selected from hydroxy, halo, cyano, S-Me, C₁₋₆alkylhalo, OC₁₋₆alkylhalo, C₁₋₆alkyl, OC₁₋₆alkyl, CO, C₀₋₆alkylcyano, C₀₋₆alkylSR⁶ and a 5-membered ring containing one or more atoms independently selected from C or O.

In yet another embodiment P is phenyl or pyridinyl and R1 is selected from Cl, F, Me, Meo, OH, CN, furyl, OCF₃,CHO, SMe and CF3.

In still a further suitable embodiment P is thiophenyl and R¹ is hydrogen. Another embodiment of invention relates to compound of formula I wherein M¹ is a bond directly between P and the 5-member ring containing X¹, X² and X³.

Embodiments of the invention include compounds of formula I where X1, X2 and X3 are each independently selected from CR, CO, N, NR, O and S. In another embodiment X¹ and X² are independently selected from the group consisting of CR, N and O and X³ is N.

In a further embodiment X3 is N, X2 is O and X1 is selected from N and C. In still another embodiment X¹ is N, X² is O and X³ is N. The ring containing X¹, X² and X³ may form an oxadiazole, isoxazole, or an oxazole.

Embodiments of the invention include those where M² is a direct bond from the 5-member ring to the variable X⁴ and those where M² is a linker group selected from C₁₋₃alkyl, C₂₋₃alkenyl, C₂₋₃alkynyl, C₀₋₄alkyl(CO)C₀₋₄alkyl, C₀₋₃alkylOC₀₋₃alkyl, C₀₋₃alkylNR⁶C₁₋₃alkyl, C₀₋₃alkyl(CO)NR⁶, C₀₋₄alkylNR⁶R⁷, C₀₋₃alkyl(SO)C₀₋₃alkyl and C₀₋₃alkyl(SO₂)C₀₋₃alkyl.

In preferred embodiments of the invention M² is selected from a bond and C₁₋₃alkyl and CO.

In another preferred embodiment M² is a bond or a methylene linker group.

When M² is not a direct bond, M² may be further substituted with 0, 1, 2 or 3, R³ groups, wherein the number of substituents R³ is designated by the term o. In a preferred embodiment o is 0, 1 or 2.

The substituent R³ may be selected from the group consisting of hydrogen, hydroxy, oxo, ═NR⁶, ═NOR⁶, C₁₋₄alkylhalo, halo, C₁₋₄alkyl, C₀₋₃alkylcycloalkyl, OC₁₋₄alkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₀₋₄alkylcyano, C₁₋₄alkylOR⁶ and C₀₋₄alkylNR⁶R⁷. In a preferred embodiment R³ is selected from hydrogen, C₁₋₄alkylhalo, C₁₋₄alkyl, C₀₋₃alkylcycloalkyl and C₀₋₄alkylcyano. Further preferred embodiments include R³ is methyl, ethyl, cyclopropyl, trifluoromethyl or cyano.

In suitable embodiments of the invention there are provided compounds of formula I where Q is a 4- to 8-membered ring or bicycle containing one or more atoms independently selected from C, N, O or S, wherein said ring or bicycle may be fused with a 5- or 6-membered ring containing one or more atoms independently selected from C, N, O or S and wherein the fused ring may be substituted by one or more A.

In suitable embodiments of the invention Q is a 6-membered ring containing one or more atoms independently selected from C and N. In another suitable embodiment Q is selected from 6 membered cycloalkyl, heterocycloalkyl, aromatic and heteroaromatic rings. Q may be a 6-membered heterocyclic ring, particularly a piperazinyl or piperidinyl ring.

In suitable embodiments of the invention the ring Q contains to variables X⁴ and X⁵, where X⁴ and X⁵ are independently selected from C, CR and N, wherein R is selected from hydrogen, C₀₋₃alkyl, halo, CO₀₋₃alkylOR⁵, CO₀₋₃alkylNR⁵R⁶, CO₀₋₃alkyl(CO)OR⁵, C₀₋₃alkylNR⁵R⁶ and C₀₋₃alkylaryl.

In a preferred embodiment of the invention X⁴ is N.

In another preferred embodiment X⁵ is C or N.

The variable X⁵ may be further substituted with 0, 1 or 2 substituents R⁵, wherein the number of substituents R⁵ is designated by the variable q.

The substituents R⁵ are selected from the group consisting of hydrogen, hydroxy, halo, oxo, C₁₋₆alkylhalo, OC₁₋₆alkylhalo, C₁₋₆alkyl, OC₁₋₆alkyl, C₀₋₆alkylC₃₋₆cycloalkyl, C₀₋₆alkylaryl, OC₀₋₆alkylaryl, (CO)R⁶, O(CO)R⁶, O(CO)OR⁶, (CO)OR⁶, C₁₋₆alkylOR⁶, OC₂₋₆alkylOR⁶, C₁₋₆alkyl(CO)R⁶, OC₁₋₆alkyl(CO)R⁶, C₀₋₆alkylCO₂R⁶, OC₁₋₆alkylCO₂R⁶, C₀₋₆alkylcyano, OC₀₋₆alkylcyano, C₀₋₆alkylNR⁶R⁷, OC₂₋₆alkylNR⁶R⁷, C₁₋₆alkyl(CO)NR⁶R⁷, C₀₋₆alkyl(CO)heteroaryl, C₀₋₆alkyl(CO)aryl, OC₁₋₆alkyl(CO)NR⁶R⁷, C₁₋₆alkyl(CO)NR⁶R⁷, C₀₋₆alkylNR⁶(CO)R⁷, OC₂₋₆alkylNR⁶(CO)R⁷, C₀₋₆alkylNR⁶(CO)NR⁶R⁷, C₁₋₆alkylNR⁶(CO)OR⁷C₀₋₆alkylSR⁶, OC₂₋₆alkylSR⁶, C₀₋₆alkyl(CO)SR⁶, C₀₋₆alkyl(CS)OR⁶ C₀₋₆alkyl(SO)R⁶, OC₁₋₆alkyl(SO)R⁶, C₀₋₆alkylSO₂R⁶, OC₀₋₆alkylSO₂R⁶, C₀₋₆alkyl(SO₂)NR⁶R⁷, OC₀₋₆alkyl(SO₂)NR⁶R⁷,C₀₋₆alkylNR⁶(SO₂)R⁷, OC₂₋₆alkylNR⁶(SO₂)R⁷, C₀₋₆alkylNR⁶(SO₂)NR⁶R⁷, OC₂₋₆alkylNR⁶(SO₂)NR⁶R⁷, (CO)NR⁶R⁷, O(CO)NR⁶R⁷, NR⁶OR⁷, NR⁶(CO)OR, SO₃R⁶ and a 5-or 6-membered ring containing one or more atoms independently selected from C, N, O or S, wherein said ring may be substituted by one or more A.

In a preferred embodiment the substituents R⁵ are selected from the group consisting of hydrogen, C₀₋₆alkylCO₂R⁶, C₀₋₆alkyl(CO)SR^{6,} C₀₋₆alkyl(CS)OR⁶ and (CO)NR⁶R⁷.

In another suitable embodiment R⁵ is (CO)OR⁶, wherein R⁶ is selected from methyl, ethyl, n-propyl i-propyl and n-butyl or R⁵ is (CO)SEt, or (CO)NMe₂, or (CO)NEt₂.

In a preferred embodiment the substituents R⁵ is selected from (CO)OMe and (CO)OEt.

In suitable embodiments of the invention the ring Q may be substituted with 1, 2, 3, or 4 substituents R⁴ wherein the number of R⁴ substituents is designated by the term p. In preferred embodiments there is one substituent R⁴.

The substituents R⁴ may be selected from the group consisting of hydrogen, hydroxy, halo, nitro, oxo, C₁₋₆alkylhalo, C₁₋₆alkyl, OC₁₋₆alkyl, C₀₋₆alkylC₃₋₆cycloalkyl, CO₀₋₆alkylaryl, OC₀₋₆alkylaryl, (CO)R⁶, O(CO)R⁶, C₁₋₆alkylOR⁶, OC₂₋₆alkylOR⁶, C₁₋₆alkyl(CO)R⁶, OC₁₋₆alkyl(CO)R⁶, C₀₋₆alkylCO₂R⁶, OC₁₋₆alkylCO₂R⁶, C₀₋₆alkylcyano, OC₁₋₆alkylcyano, C₀₋₆alkylNR⁶R⁷, OC₂₋₆alkylNR⁶R⁷, C₀₋₆alkyl(CO)NR⁶R⁷, OC₀₋₆alkyl(CO)NR⁶R⁷, C₀₋₆alkylNR⁶(CO)R⁷, OC₂₋₆alkylNR⁶ (CO)R⁷, C₀₋₆alkylNR⁶(CO)NR⁶R⁷, C₀₋₆alkylSR⁶, OC₂₋₆alkylSR⁶, C₀₋₆alkyl(SO)R⁶, OC₂₋₆alkyl(SO)R⁶, C₀₋₆alkylSO₂R⁶, OC₀₋₆alkylSO₂R⁶, C₀₋₆alkyl(SO₂)NR⁶R⁷, OC₀₋₆alkyl(SO₂)NR⁶R⁷, C₀₋₆alkylNR⁶(SO₂)R⁷, OC₂₋₆alkylNR⁶(SO₂)R⁷, NR⁶OR⁷, NR⁶(CO)OR⁷, SO₃R⁶ and a 5- or 6-membered ring containing one or more atoms independently selected from C, N, O or S, wherein said ring may be fused with a 5- or 6-membered ring containing one or more atoms independently selected from C, N, O or S and wherein said ring and said fused ring may be substituted by one or more A.

In preferred embodiments R⁴ is selected from hydrogen, oxo, C₁₋₆alkyl, C₀₋₆alkylCO₂R⁶ and a 6-membered ring containing one or more atoms independently selected from C, N or O, wherein said ring may be fused with phenyl and wherein said ring may be substituted by one or more A and R⁶ is C₁₋₆alkyl. In a suitable embodiment R⁴ is selected from hydrogen, oxo, methyl, ethylcarboxy and dihydro-benzo-oxazin-one.

In more preferred embodiments R⁴ is selected from hydrogen and methyl.

Furthermore, any C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl defined under R¹, R², R³, R⁴, R⁵, R⁶ and R⁷ may be substituted by one or more A and A may be selected from the group consisting of hydrogen, hydroxy, oxo, halo, nitro, C₁₋₆alkylhalo, OC₁₋₆alkylhalo, C₁₋₆alkyl, C₀₋₄alkylC₃₋₆cycloalkyl, C₂₋₆alkenyl, OC₁₋₆alkyl, C₀₋₃alkylaryl, C₁₋₆alkylOR⁶, OC₂₋₆alkylOR⁶, C₁₋₆alkylSR⁶, OC₂₋₆alkylSR⁶, (CO)R⁶, O(CO)R⁶, OC₂₋₆alkylcyano, C₀₋₆alkylcyano, C₀₋₆alkylCO₂R⁶, OC₁₋₆alkylCO₂R⁶, O(CO)OR⁶, OC₁₋₆alkyl(CO)R⁶, C₁₋₆alkyl(CO)R⁶, NR⁶OR⁷, C₀₋₆alkylNR⁶R⁷, OC₂₋₆alkylNR⁶R⁷, C₀₋₆alkyl(CO)NR⁶R⁷, OC₁₋₆alkyl(CO)NR⁶R⁷, OC₂₋₆alkylNR⁶(CO)R⁷, C₀₋₆alkylNR⁶(CO)R⁷, C₀₋₆alkylNR⁶(CO)NR⁶R⁷, O(CO)NR⁶R⁷, NR⁶(CO)OR⁷, C₀₋₆alkyl(SO₂)NR⁶R⁷, OC₂₋₆alkyl(SO₂)NR⁶R⁷, C₀₋₆alkylNR⁶(SO₂)R⁷, OC₂₋₆alkylNR⁶(SO₂)R⁷, SO₃R⁶, C₁₋₆alkylNR⁶(SO₂)NR⁶R⁷, OC₂₋₆alkyl(SO₂)R⁶, C₀₋₆alkyl(SO₂)R⁶, C₀₋₆alkyl(SO)R⁶ and OC₂₋₆alkyl(SO)R⁶.

In a preferred embodiment A is selected form hydrogen, oxo and NR⁶(CO)OR⁷. In a suitable embodiment of the invention R⁴ is substituted with A, wherein A is oxo or NR⁶(CO)OR⁷, and wherein R⁶ and R⁷ are C₁₋₂alkyl.

In a more suitable embodiment of the invention ring Q may be substituted with ethoxyamidomethyl or dihydro-benzo-oxazin-one.

Further examples of compounds of formula I are compounds wherein:
P is selected from the group consisting of C₃₋₇alkyl and a 3- to 8-membered ring containing one or more atoms independently selected from C, N, O or S, wherein said ring may be fused with a 5- or 6-membered ring containing one or more atoms independently selected from C, N, O or S;
R¹ is selected from the group consisting of hydrogen, hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, (CO)R⁶, O(CO)R⁶, O(CO)OR⁶, C₁₋₆alkylOR⁶, OC₂₋₆alkylOR⁶, C₁₋₆alkyl(CO)R⁶, OC₁₋₆alkyl(CO)R⁶, C₀₋₆alkylCO₂R⁶, OC₁₋₆alkylCO₂R⁶, C₀₋₆alkylcyano, OC₂₋₆alkylcyano, C₀₋₆alkylNR⁶R⁷, OC₂₋₆alkylNR⁶R⁷, C₁₋₆alkyl(CO)NR⁶R⁷, OC₁₋₆alkyl(CO)NR⁶R⁷, C₀₋₆alkylNR⁶(CO)R⁷, OC₂₋₆alkylNR⁶(CO)R⁷, C₀₋₆alkylNR⁶(CO)NR⁶R⁷, C₀₋₆alkylSR⁶, OC₂₋₆alkylSR⁶, C₀₋₆alkyl(SO)R⁶, OC₂₋₆alkyl(SO)R⁶, C₀₋₆alkylSO₂R⁶, OC₂₋₆alkylSO₂R⁶, C₀₋₆alkyl(SO₂)NR⁶R⁷, OC₂₋₆alkyl(SO₂)NR⁶R⁷, C₀₋₆alkylNR⁶(SO₂)R⁷, OC₂₋₆alkylNR⁶(SO₂)R⁷, C₀₋₆alkylNR⁶(SO₂)NR⁶R⁷, OC₂₋₆alkylNR⁶(SO₂)NR⁶R⁷, (CO)NR⁶R⁷, O(CO)NR⁶R⁷, NR⁶OR⁷, C₀₋₆alkylNR⁶(CO)OR⁷, OC₂₋₆alkylNR⁶(CO)OR⁷, SO₃R⁶ and a 5- or 6-membered ring containing one or more atoms independently selected from C, N, O or S, wherein said ring may be substituted by one or more A;
M¹ is selected from the group consisting of a bond, C₁₋₃alkyl, C₂₋₃alkenyl, C₂₋₃alkynyl, C₀₋₄alkyl(CO)C₀₋₄alkyl, C₀₋₃alkylOC₀₋₃alkyl, C₀₋₃alkyl(CO)NR⁷R⁶, C₀₋₃alkyl(CO)NR⁷R⁶C₁₋₃alkyl, C₀₋₄alkylNR⁷R⁶, C₀₋₃alkylSC₀₋₃alkyl, C₀₋₃alkyl(SO)C₀₋₃alkyl and C₀₋₃alkyl(SO₂)C₀₋₃alkyl;
X¹, X² and X³ are independently selected from the group consisting of CR, CO, N, NR, O and S;
R is selected from the group consisting of hydrogen, C₀₋₃alkyl, halo, C₀₋₃alkylOR⁵, C₀₋₃alkylNR⁵R⁶, C₀₋₃alkyl(CO)OR⁵, C₀₋₃alkylNR⁵R⁶ and C₀₋₃alkylaryl;
R² is selected from the group consisting of hydrogen, hydroxy, oxo, ═NR⁶, ═NOR⁶, C₁₋₄alkylhalo, halo, C₁₋₄alkyl, OC₁₋₄alkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₁₋₄alkyl, C₀₋₄alkylcyano, C₁₋₄alkylOR⁶ and C₀₋₄alkylNR⁶R⁷;
M² is selected from the group consisting of a bond, C₁₋₃alkyl, C₂₋₃alkenyl, C₂₋₃alkynyl, C₀₋₄alkyl(CO)C₀₋₄alkyl, C₀₋₃alkylOC₀₋₃alkyl, C₀₋₃alkylNR⁶C₁₋₃alkyl, C₀₋₃alkyl(CO)NR⁶, C₀₋₄alkylNR⁶R⁷, C₀₋₃alkylSC₀₋₃alkyl, C₀₋₃alkyl(SO)C₀₋₃alkyl and C₀₋₃alkyl(SO₂)C₀₋₃alkyl;
R³ is selected from the group consisting of hydrogen, hydroxy, oxo, ═NR⁶, ═NOR⁶, C₁₋₄alkylhalo, halo, C₁₋₄alkyl, OC₁₋₄alkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₀₋₄alkylcyano, C₁₋₄alkylOR⁶ and C₀₋₄alkylNR⁶R⁷;
X⁴ is selected from C, CR or N;
X⁵ is selected from C, CR or N;
Q is a 4- to 8-membered ring or bicycle containing one or more atoms independently selected from C, N, O or S, wherein said ring or bicycle may be fused with a 5- or 6-membered ring containing one or more atoms independently selected from C, N, O or S and wherein the fused ring may be substituted by one or more A;
R⁴ is selected from the group consisting of hydrogen, hydroxy, halo, nitro, oxo, C₁₋₆alkylhalo, C₁₋₆alkyl, OC₁₋₆alkyl, C₀₋₆alkylC₃₋₆cycloalkyl, C₀₋₆alkylaryl, OC₀₋₆alkylaryl, (CO)R⁶, O(CO)R⁶, C₁₋₆alkylOR⁶, OC₂₋₆alkylOR⁶, C₁₋₆alkyl(CO)R⁶, OC₁₋₆alkyl(CO)R⁶, C₀₋₆alkylCO₂R⁶, OC₁₋₆alkylCO₂R⁶, C₀₋₆alkylcyano, OC₁₋₆alkylcyano, C₀₋₆alkylNR⁶R⁷, OC₂₋₆alkylNR⁶R⁷, C₀₋₆alkyl(CO)NR⁶R⁷, OC₀₋₆alkyl(CO)NR⁶R⁷, C₀₋₆alkylNR⁶(CO)R⁷, OC₂₋₆alkylNR⁶(CO)R⁷, C₀₋₆alkylNR⁶(CO)NR⁶R⁷, C₀₋₆alkylSR⁶, OC₂₋₆alkylSR⁶, C₀₋₆alkyl(SO)R⁶, OC₂₋₆alkyl(SO)R⁶, C₀₋₆alkylSO₂R⁶, OC₀₋₆alkylSO₂R⁶, C₀₋₆alkyl(SO₂)NR⁶R⁷, OC⁰⁻⁶alkyl(SO₂)NR⁶R⁷, C₀₋₆alkylNR⁶(SO₂)R⁷, OC₂₋₆alkylNR⁶(SO₂)R⁷, NR⁶OR⁷, NR⁶(CO)OR⁷, SO₃R⁶ and a 5- or 6-membered ring containing one or more atoms independently selected from C, N, O or S, wherein said ring may be substituted by one or more A;
R⁵ is selected from the group consisting of hydrogen, hydroxy, halo, oxo, C₁₋₆alkylhalo, OC₁₋₆alkylhalo, C₁₋₆alkyl, OC₁₋₆alkyl, C₀₋₆alkylC₃₋₆cycloalkyl, C₀₋₆alkylaryl, OC₀₋₆alkylaryl, (CO)R⁶, O(CO)R⁶, O(CO)OR⁶, (CO)OR⁶, C₁₋₆alkylOR⁶, OC₂₋₆alkylOR⁶, C₁₋₆alkyl(CO)R⁶, OC₁₋₆alkyl(CO)R⁶, C₀₋₆alkylCO₂R⁶, OC₁₋₆alkylCO₂R⁶, C₀₋₆alkylcyano, OC₀₋₆alkylcyano, C₀₋₆alkylNR⁶R⁷, OC₂₋₆alkylNR⁶R⁷, C₁₋₆alkyl(CO)NR⁶R⁷, CO₀₋₆alkyl(CO)heteroaryl, C₀₋₆alkyl(CO)aryl, OC₁₋₆alkyl(CO)NR⁶R⁷, C₁₋₆alkyl(CO)NR⁶R⁷, C₀₋₆alkylNR⁶(CO)R⁷, OC₂₋₆alkylNR⁶(CO)R⁷, C₀₋₆alkylNR⁶(CO)NR⁶R⁷, C₁₋₆alkylNR⁶(CO)OR⁷ C₀₋₆alkylSR⁶, OC₂₋₆alkylSR⁶, C₀₋₆alkyl(SO)R⁶, OC₁₋₆alkyl(SO)R⁶, C₀₋₆alkylSO₂R⁶, OC₀₋₆alkylSO₂R⁶, C₀₋₆alkyl(SO₂)NR⁶R⁷, OC₀₋₆alkyl(SO₂)NR⁶R⁷,C₀₋₆alkylNR⁶(SO₂)R⁷, OC₂₋₆alkylNR⁶(SO₂)R⁷, C₀₋₆alkylNR⁶(SO₂)NR⁶R⁷, OC₂₋₆alkylNR⁶(SO₂)NR⁶R⁷, (CO)NR⁶R⁷, O(CO)NR⁶R⁷, NR⁶OR⁷, NR⁶(CO)OR⁷, SO₃R⁶ and a 5-or 6-membered ring containing one or more atoms independently selected from C, N, O or S, wherein said ring may be substituted by one or more A;
R⁶ and R⁷ are independently selected from hydrogen, C₁₋₆alkyl, C₀₋₆alkylC₃₋₆cycloalkyl, C₀₋₆alkylaryl, C₁₋₆alkylheteroaryl and a 5- or 6-membered ring containing one or more atoms independently selected from C, N, O or S, and wherein R⁶ and R⁷ may together form a 5- or 6-membered ring containing one or more atoms independently selected from C, N, O or S;
wherein any C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl defined under R¹, R², R³, R⁴, R⁵, R⁶ and R⁷ may be substituted by one or more A;
A is selected from the group consisting of hydrogen, hydroxy, oxo, halo, nitro, C₁₋₆alkylhalo, OC₁₋₆alkylhalo, C₁₋₆alkyl, C₀₋₄alkylC₃₋₆cycloalkyl, C₂₋₆alkenyl, OC₁₋₆alkyl, C₀₋₃alkylaryl, C₁₋₆alkylOR⁶, OC₂₋₆alkylOR⁶, C₁₋₆alkylSR⁶, OC₂₋₆alkylSR⁶, (CO)R⁶, O(CO)R⁶, OC₂₋₆alkylcyano, C₀₋₆alkylcyano, C₀₋₆alkylCO₂R⁶, OC₁₋₆alkylCO₂R⁶, O(CO)OR⁶, OC₁₋₆alkyl(CO)R⁶, C₁₋₆alkyl(CO)R⁶, NR⁶ OR⁷, C₀₋₆alkylNR⁶R⁷, OC₂₋₆alkylNR⁶R⁷, C₀₋₆alkyl(CO)NR⁶R⁷, OC₁₋₆alkyl(CO)NR⁶R⁷, OC₂₋₆alkylNR⁶(CO)R⁷, C₀₋₆alkylNR⁶(CO)R⁷, C₀₋₆alkylNR⁶(CO)NR⁶R⁷, O(CO)NR⁶R⁷, NR⁶(CO)OR⁷, C₀₋₆alkyl(SO₂)NR⁶R⁷, OC₂₋₆alkyl(SO₂)NR⁶R⁷, C₀₋₆alkylNR⁶(SO₂)R⁷, OC₂₋₆alkylNR⁶(SO₂)R⁷, SO₃R⁶, C₁₋₆alkylNR⁶(SO₂)NR⁶R⁷, OC₂₋₆alkyl(SO₂)R⁶, C₀₋₆alkyl(SO₂)R⁶, C₀₋₆alkyl(SO)R⁶ and OC₂₋₆alkyl(SO)R⁶;
m is selected from 0, 1, 2, 3 or 4; and
n is selected from 0, 1, 2 or 3;
or salt thereof.

The present invention relates to the use of compounds of formula I as hereinbefore defined as well as to the salts thereof. Salts for use in pharmaceutical formulations will be pharmaceutically acceptable salts, but other salts may be useful in the production of the compounds of formula I.

Examples of pharmaceutically acceptable salts may be, but are not limited to hydrochloride, 4-aminobenzoate, anthranilate, 4-aminosalicylate, 4-hydroxybenzoate, 3,4-dihydroxybenzoate, 3-hydroxy-2-naphthoate, nitrate and trifluoroacetate. Other pharmaceutically acceptable salts and methods of preparing these salts may be found in, for example, Remington's Pharmaceutical Sciences (18^{th} Edition, Mack Publishing Co.).

Some compounds of formula I may have chiral centres and/or geometric isomeric centres (E- and Z-isomers), and it is to be understood that the invention encompasses all such optical, diastereoisomers and geometric isomers.

The invention relates to any and all tautomeric forms of the compounds of formula I.

The invention further relates to solvate or hydrate forms of compounds of formula 1. The term solvate as used here refers to a compound of formula 1 wherein molecules of a suitable solvent are incorporated in the crystal lattice. One example of a suitable solvent is ethanol. The term hydrate as used here refers to a compound of formula 1 wherein molecules of water are incorporated in the crystal lattice.

The invention relates to the following compounds, which may be used as intermediates in the preparation of a compound of formula I;
N,N-Bis-(2-trifluoromethanesolfonyl-ethyl)-2-nitrobenzenesulfonamide,
(Cyano-methyl-methyl)-carbamic acid tert-butyl ester,
2-Chloro-N-hydroxy-acetamidine,
[1-(N-Hydroxycarbamimidoyl)-ethyl]-1-carbamic acid tert-butyl ester,
3-Chloromethyl-5-m-tolyl-[1,2,4]oxadiazole,
3-(3-Chloromethyl-[1,2,4]oxadiazol-5-yl)-benzonitrile,
3-Chloromethyl-5-(3-fluoro-phenyl)-[1,2,4]oxadiazole,
3-Chloromethyl-5-(3-iodo-phenyl)-[1,2,4]oxadiazole,
3-Chloromethyl-5-(3-chloro-phenyl)-[1,2,4]oxadiazole,
3-Chloromethyl-5-(3-trifluoromethoxy-phenyl)-[1,2,4]oxadiazole,
5-(3-Bromo-phenyl)-3-chloromethyl-[1,2,4]oxadiazole,
1-(5-(3-Methylphenyl-[1,2,4]oxadiazol-3-yl)-ethylamine,
1-[1-(5-(3-Methyl-phenyl)-[1,2,4]oxadiazol-3-yl)-ethyl]-piperazine,
1-(5-m-Tolyl-[1,2,4]oxadiazol-3-ylmethyl)-piperazine or
1-[5-(3-Methoxy-phenyl)-[1,2,4]oxadiazol-3-ylmethyl]-3-methyl-piperazine.
   Pharmaceutical Formulations

According to one aspect of the present invention there is provided a pharmaceutical formulation comprising a compound of formula I, or salt thereof, for use in the prevention and/or treatment of metabotropic glutamate receptor subtype 5 receptor (mGluR5) mediated disorders and any disorder listed below.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration as an ointment, patch or cream or for rectal administration as a suppository.

In general the above compositions may be prepared in a conventional manner using one or more conventional excipients, pharmaceutical diluents and/or inert carriers.

According to another aspect of the invention there is provided a pharmaceutical formulation comprising as active ingredient a therapeutically effective amount of a compound of formula I in association with one or more pharmaceutically acceptable diluent, excipients and/or inert carrier.

Suitable daily doses of the compounds of formula I in the treatment of a mammal, including man are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

Medical Use

It has been found that the compounds according to the present invention, or salts thereof, exhibit a high degree of potency and selectivity for individual metabotropic glutamate receptor (mGluR) subtypes. In particular there are compounds according to the present invention that are potent and selective for the mGluR Group I receptor and more particularly for mGluR5. Accordingly, the compounds of the present invention are expected to be useful in the prevention and/or treatment of conditions associated with excitatory activation of an mGluR Group I receptor and for inhibiting neuronal damage caused by excitatory activation of an mGluR Group I receptor, specifically when the mGluR Group I receptor is mGluR5. The compounds may be used to produce an inhibitory effect of mGluR Group I, especially mGluR5, in mammals, including man.

mGluR5 is highly expressed in the central and peripheral nervous system and in other tissues. Thus, it is expected that the compounds of the invention are well suited for the prevention and/or treatment of mGluR5 receptor-mediated disorders such as acute and chronic neurological and psychiatric disorders and chronic and acute pain disorders.

Further disorders are Alzheimer's disease senile dementia, AIDS-induced dementia, Parkinson's disease, amyotrophic lateral sclerosis, Huntington's Chorea, migraine, epilepsy, schizophrenia, depression, anxiety, acute anxiety, obsessive compulsive disorder, ophthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, addiction and craving disorders, neurodevelopmental disorders including Fragile X, autism, mental retardation, schizophrenia and Down's Syndrome.

The compounds are also well suited for the prevention and/or treatment of pain related to migraine, inflammatory pain, neuropathic pain disorders such as diabetic neuropathies, arthritis and rheumatitiod diseases, low back pain, post-operative pain and pain associated with various conditions including angina, renal or billiary colic, menstruation, migraine and gout.

Other disorders are stroke, head trauma, anoxic and ischemic injuries, hypoglycemia, cardiovascular diseases and epilepsy.

The dose required for the therapeutic or preventive treatment of a particular disorder will necessarily be varied depending on the host treated, the route of administration and the severity of the illness being treated.

The invention relates to compounds of formula I as defined hereinbefore, for use in therapy.

The invention relates to compounds of formula I as defined hereinbefore, for use in prevention and/or treatment of neurological disorders.

The invention relates to compounds of formula I as defined hereinbefore, for use in prevention and/or treatment of psychiatric disorders.

The invention relates to compounds of formula I as defined hereinbefore, for use in prevention and/or treatment of chronic and acute pain disorders.

The invention relates to compounds of formula I as defined hereinbefore, for use in prevention and/or treatment of mGluR5 receptor-mediated disorders.

The invention relates to compounds of formula I as defined hereinbefore, for use in prevention and/or treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, ophthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of formula I as defined hereinbefore, for use in prevention and/or treatment of pain related to migraine, inflammatory pain, neuropathic pain disorders such as diabetic neuropathies, arthritis and rheumatitiod diseases, low back pain, post-operative pain and pain associated with various conditions including angina, renal or billiary colic, menstruation, migraine and gout.

The invention relates to compounds of formula I as defined hereinbefore, for use in prevention and/or treatment of stroke, head trauma, anoxic and ischemic injuries, hypoglycemia, cardiovascular diseases and epilepsy.

The present invention relates also to the use of a compound of formula I as defined hereinbefore, in the manufacture of a medicament for the prevention and/or treatment of mGluR5 receptor-mediated disorders and any disorder listed above.

The invention also provides a method of treatment and/or prevention of mGluR5 receptor-mediated disorders and any disorder listed above, in a patient suffering from, or at risk of, said condition, which comprises administering to the patient an effective amount of a compound of formula I, as hereinbefore defined.

In the context of the present specification, the term “therapy” includes treatment as well as prevention, unless there are specific indications to the contrary. The terms “therapeutic” and “therapeutically” should be construed accordingly.

In this specification, unless stated otherwise, the term ‘antagonist’ means a compound that by any means, partly or completely, blocks the transduction pathway leading to the production of a response by the ligand.

The term “disorder”, unless stated otherwise, means any condition and disease associated with metabotropic glutamate receptor activity.

Non-Medical Use

In addition to their use in therapeutic medicine, the compounds of formula I or salt thereof, are also useful as pharmacological tools in the development and standardisation of in vitro and in vivo test systems for the evaluation of the effects of inhibitors of mGluR related activity in laboratory animals such as cats, dogs, rabbits, monkeys, rats and mice, as part of the search for new therapeutics agents.

Pharmacology

The pharmacological properties of the compounds of the invention can be analyzed using standard assays for functional activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay that measures the mobilization of intracellular calcium, [Ca²⁺]ᵢ in cells expressing mGluR5.

Intracellular calcium mobilization was measured by detecting changes in fluorescence of cells loaded with the fluorescent indicator fluo-3. Fluorescent signals were measured using the FLIPR system (Molecular Devices). A two addition experiment was used that could detect compounds that either activate or antagonize the receptor.

For FLIPR analysis, cells expressing human mGluR5d were seeded on collagen coated clear bottom 96-well plates with black sides and analysis of [Ca^{2+]}ᵢ mobilization was done 24 hours after seeding.

FLIPR experiments were done using a laser setting of 0.800 W and a 0.4 second CCD camera shutter speed. Each FLIPR experiment was initiated with 160 μL of buffer present in each well of the cell plate. After each addition of the compound, the fluorescence signal was sampled 50 times at 1 second intervals followed by 3 samples at 5 second intervals. Responses were measured as the peak height of the response within the sample period.

EC₅₀ and IC₅₀ determinations were made from data obtained from 8-point concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

We have validated a secondary functional assay for mGluR5d based on Inositol Phosphate (IP₃) turnover. IP₃ accumulation is measured as an index of receptor mediated phospholipase C turnover. GHEK cells stably expressing the human mGluR5d receptors were incubated with [3H] myo-inositol overnight, washed three times in HEPES buffered saline and pre-incubated for 10 minutes with 10 mM LiCl. Compounds (agonists) were added and incubated for 30 minutes at 37° C. Antagonist activity was determined by pre-incubating test compounds for 15 minutes, then incubating in the presence of glutamate (80 μM) or DHPG (30 μM) for 30 minutes. Reactions were terminated by the addition of perchloric acid (5%). Samples were collected and neutralized, and inositol phosphates were separated using Gravity-Fed Ion-Exchange Columns.

A detailed protocol for testing the compounds of the invention is provided below in Pharmaceutical Examples.

One aspect of the invention relates to a method for inhibiting activation of mGluR5 receptors, comprising treating a cell containing said receptor with an effective amount of a compound of formula I.

### Abbreviations

FLIPR Fluorometric Imaging Plate reader
CCD Charge Coupled Device
CRC Concentration Response Curve
GHEK Human Embrionic Kidney expressing Glutamate Transporter
HEPES 4-(2-hydroxyethyl)-1-piperazineethanesulfonic acid (buffer)
IP₃ inositol triphosphate
DHPG 3,5-dihydroxyphenylglycine;
BSA Bovine Serum Albumin
EDTA Ethylene Diamine Tetraacetic Acid
DIPEA N-Ethyldiisopropylamine
TBAF Tetrabutylammonium fluoride
   Methods of Preparation

Another aspect of the present invention provides a process for preparing a compound of formula I or salt thereof.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, 1999.

Throughout the following description of such processes it is to be understood that cross-couplings can be performed in a manner that will be readily understood by one skilled in the art of organic synthesis. Conventional procedures for cross-coupling are described, for example, in “Organicmetallics in Syntheses”, M. Schlosser (Ed.), John Wiley and Sons (year)

Unless specified otherwise, P, Q, X¹, X², X³, X⁴, X⁵, R, R¹, R², R³, R⁴, R⁵, R⁶, R⁷, m, n, o, p and q are defined as in formula I.

All starting materials are commercially available or earlier described in the literature. The ¹H and ¹³C NMR spectra were recorded on one of a Bruker 300 at 300 MHz Bruker, DPX400 at 400 MHz or Varian +400 spectrometer at 100 MHz, using TMS or the residual solvent signal as reference.

Mass spectra were recorded on a QTOF Global Micromass or a Waters LCMS consisting of an Alliance 2795 (LC) and a ZQ single quadropole mass spectrometer. The mass spectrometer was equipped with an electrospray ion source operated in a positive or negative ion mode. The ion spray voltage was ±3 kV and the mass spectrometer was scanned from m/z 100-700 with a scan time of 0.8 s. Column: X-Terra MS, Waters, C8, 2.1×50 mm, 3.5 μm and the column temperature was set to 40° C. A linear gradient was applied, run at 0% to 100% acetonitrile in 4 minutes, flow rate 0.3 ml/min. Mobile phase: acetonitrile/10 mM ammonium acetate in 5% acetonitrile in MilliQ Water.

Preparative chromatography was run on a Gilson autopreparative HPLC with a diode array detector. Column: XTerra MS C8, 19×300 mm, 7 μm. Gradient with acetonitrile/0.1M ammonium acetate in 5% acetonitrile in MilliQ Water, generally run from 20% to 60% acetonitrile, in 13 min. Flowrate: 20 ml/min.

MS-triggered prep-LC was run on a Waters autopurification LC-MS system with a diode array detector and a ZQ mass detector. Column: XTerra MS C8, 19×100 mm, 5 μm. Gradient with acetonitrile/0.1M ammonium acetate in 5% acetonitrile in MilliQ Water, run from 0% to 100% acetonitrile, in 10 min. Flowrate: 20 ml/min.

In some cases purification by a chromatotron was performed on rotating silica gel/gypsum (Merck, 60 PF-254 with calcium sulphate) coated glass sheets, with coating layer of 2 mm using a TC Research 7924T chromatotron. Alternatively Chem Elut Extraction Column (Varian, cat #1219-8002) and Mega BE-SI (Bond Elut Silica) SPE Columns (Varian, cat # 12256018; 12256026; 12256034) were used during purification of the products.

The microwave heating was performed in a Smith Synthesizer Single-mode microwave cavity producing continuous irradiation at 2450 MHz (Personal Chemistry AB, Uppsala, Sweden).

### Abbreviations

DMF N,N-dimethylformamide
EDCI 1-(3-dimethylaminopropyl)-3-ethylcarbodiimide hydrochloride
HOBt 1-hydroxybenzotriazole hydrate
THF tetrahydrofuran
TFA trifluoroacetic acid
Et ethyl
Ac acetyl
DIBAL diisobutylaluminum hydride
M, N molar and normal
HBTU O-Benzotriazol-1-yl-N,N,N′,N′-tetramethyluronium hexafluorophosphate
Boc tert-butoxycarbonyl
MCPBA meta-chloroperoxybenzoic acid
SPE solid phase extraction
   General Syntheses of Compounds of Formula V

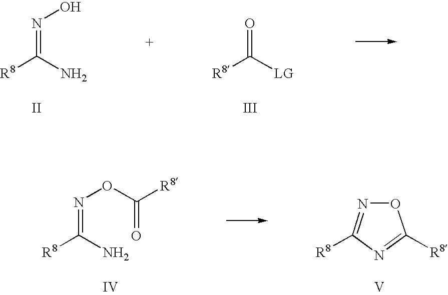

A compound of formula V, wherein R⁸ and R⁸′ are independently selected from a group consisting of M¹-(R²)ₙ—P—(R¹)ₘ or M² (R³)ₙ-Q(R⁴)ₘ-R⁵ or M² (R³)ₙ-G², wherein LG² is a leaving group such as chloro or mesylate, or a chemical functional group which may subsequently be transformed into M²(R³)ₙ-Q(R⁴)ₘ-R⁵, may be prepared through cyclization of a compound of formula IV, which in turn may be formed from a suitably activated compound of formula III with a compound of formula II.

Compounds of formula II may be prepared from a suitable nitrile, or from a suitably substituted cyanamide in the case where M² is a bond and X⁴ is N, by addition of hydroxylamine, for example as the hydrochloride salt, in a suitable solvent such as, methanol, ethanol, water, dioxane or mixture thereof, using an appropriate base such as hydroxide, carbonate, acetate, or pyrdine. Compound of formula II wherein R⁸ is M² (R³)ₙ-Q(R⁴)ₘ-R⁵ and Q(R⁴)ₘ-R⁵ contains a suitable nucleophilic residue, may be formed via nucleophilic displacement using a compound of formula II wherein R⁸ is M²(R³)ₙLG².

The compound of formula III may be activated in the following non-limiting ways: i) as the acid chloride formed from the acid using a suitable reagent such as oxalyl chloride or thionyl chloride; ii) as an anhydride or mixed anhydride formed from treatment with a reagent such as alkyl chloroformate; iii) using traditional methods to activate acids in amide coupling reactions such as EDCI with HOBt or uronium salts like HBTU; iv) as an alkyl ester when the hydroxyamidine is deprotonated using a strong base like sodium tert-butoxide or sodium hydride in a solvent such as ethanol or toluene at elevated temperatures (80-110° C.).

This transformation of compounds II and III into compounds of type V may be performed as two consecutive steps via an isolated intermediate of type IV, as described above, or the cyclization of the intermediate formed in situ may occur spontaneously during the ester formation. The formation of ester IV may be accomplished using an appropriate aprotic solvent such as dichloromethane, tetrahydrofuran, N,N-dimethylformamide or toluene, with optionally an appropriate organic base such as triethylamine, diisopropylethylamine and the like or an inorganic base such sodium bicarbonate or potassium carbonate. The cyclization of compounds of formula IV to form an oxadiazole may be carried out on the crude ester with evaporation and replacement of the solvent with a higher boiling solvent such as DMF or with aqueous extraction to provide a semi-purified material or with material purified by standard chromatographic methods. The cyclization may be accomplished by heating conventionally or by microwave irradiation (100-180° C.), in a suitable solvent such as pyridine or N,N-dimethylformamide or using a lower temperature method employing reagents like tetrabutylammonium fluoride in tetrahydrofuran or by any other suitable known literature method.

Further examples of the above described reactions can be found in Poulain et al., Tetrahedron Lett., (2001), 42, 1495-98, Ganglott et al., Tetrahedron Lett., (2001), 42, 1441-43, and Mathvink et al, Bioorg. Med. Chem. Lett. (1999), 9, 1869-74, which are hereby included as references

Synthesis of Nitriles and Acids for use in Preparation of Compounds of Formula II & III

Substituted cyanamides, for use in the formation of compounds of formula II wherein M² is a bond and X⁴ is N, may be commercially available or may be formed by treatment of an suitably substituted amine with a cyanogen halide in a suitable solvent such as diethyl ether.

Aryl nitriles are available by a variety of methods including cyanation of an aryl halide or triflate under palladium or nickel catalysis using an appropriate cyanide source such as zinc cyanide in an appropriate solvent such as N,N-dimethylformamide. The corresponding acid is available from the nitrile by hydrolysis under either acidic or basic conditions in an appropriate solvent such as aqueous alcohols. Aryl acids are also available from a variety of other sources, including iodo- or bromo-lithium exchange followed by trapping with CO₂ to give directly the acid.

Carboxylic acids may be converted to primary amides using any compatible method to activate the acid, including via the acid chloride or mixed anhydride, followed by trapping with any source of ammonia, including ammonium chloride in the presence of a suitable base, ammonium hydroxide, methanolic ammonia or ammonia in an aprotic solvent such as dioxane. This amide intermediate may be converted to the nitrile using a variety of dehydration reagents such as oxalyl chloride or thionyl chloride. This reaction sequence to convert an acid into a nitrile may also be applied to non-aromatic acids, including suitably protected amino acid derivatives. A suitable protecting group for an amine, in an amino acid or in a remote position of any other acid starting material, may be any group which removes the basicity and nucleophilicity of the amine functionality, including such carbamate protecting group as Boc.

Some acids are more easily prepared taking advantage of commercially available analogs. For example, 6-methylpyridine-4-carboxylic acid is prepared by dechlorination of 2-chloro-6-methylpyridine-4-carboxylic acid. Certain types of substituted fluoro-benzonitriles and benzoic acids are available from bromo-difluoro-benzene via displacement of one fluoro group with a suitable nucleophile such as imidazole in the presence of a base such as potassium carbonate in a compatible solvent such as NAN-dimethylformamide at elevated temperatures (80-120° C.) for extended periods of time. The bromo group may subsequently be elaborated into the acid or nitrile as above.

1,3-Disubsituted and 1,3,5-trisubstituted benzoic acids and benzonitriles may be prepared by taking advantage of readily available substituted isophthalic acid derivatives. Monohydrolysis of the diester allows selective reaction of the acid with a variety of reagents, most typically activating agents such as thionyl chloride, oxalyl chloride or isobutyl chloroformate and the like. From the activated acid, a number of products are available. In addition to the primary amide used to form the nitrile by dehydration as mentioned above, reduction to the hydroxymethyl analog may be carried out on the mixed anhydride or acid chloride using a variety of reducing agents such as sodium borohydride in a compatible solvent such as tetrahydrofuran. The hydroxymethyl derivative may be further reduced to the methyl analog using catalytic hydrogenation with an appropriate source of catalyst such as palladium on carbon in an appropriate solvent such as ethanol. The hydroxymethyl group may also be used in any reaction suitable for benzylic alcohols such as acylation, alkylation, transformation to halogen and the like. Halomethylbenzoic acids of this type may also be obtained from bromination of the methyl derivative when not commercially available. Ethers obtained by alkylation of the hydroxymethyl derivatives may also be obtained from the halomethylaryl benzoate derivatives by reaction with the appropriate alcohol using an appropriate base such as potassium carbonate or sodium hydroxide in an appropriate solvent such as tetrahydrofuran or the alcohol. When other substituents are present, these may also be employed in standard transformation reactions. Treatment of an aniline with acid and sodium nitrite may yield a diazonium salt, which may be transformed into a halide such as fluoride using tetrafluoroboric acid. Phenols react in the presence of a suitable base such as potassium carbonate with alkylating agents to form aromatic ethers.
Formation of compounds of formula IX

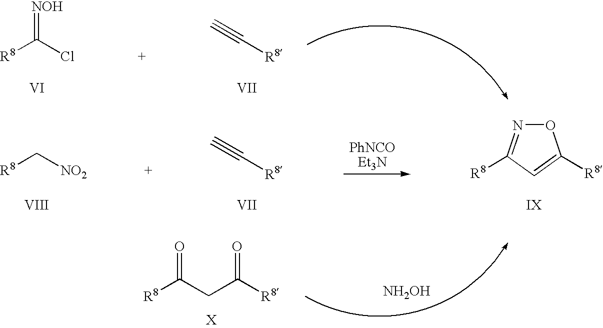

A compound of formula IX, wherein R⁸ and R⁸′ are independently selected from a group consisting of M¹-(R²)ₙ-P-(R¹)ₘ or M²(R³)ₙ-Q(R⁴)ₘ-R⁵ or M² (R³)ₙLG² or a chemical functional group which may subsequently be transformed into M²(R³)ₙ-Q(R⁴)ₘ-R⁵, may be prepared by a 1,3-dipolar cycloaddition between compounds of formula VI and VII under basic conditions using a suitable base such as sodium bicarbonate or triethylamine at suitable temperatures (0° C.-100° C.) in solvents such as toluene. Synthesis of compounds of type VI has previously been described in the literature, e.g. Kim, Jae Nyoung; Ryu, Eung K; J. Org. Chem. (1992), 57, 6649-50. 1,3-Dipolar cycloaddition with acetylenes of type VII can also be effected using substituted nitromethanes of type VIII via activation with an electrophilic reagent such as PhNCO in the presence of a base such as triethylamine at elevated temperatures (50-100° C.). Li, C-S.; Lacasse, E.; Tetrahedron Lett. (2002) 43; 3565-3568. Several compounds of type VII are commercially available, or may be synthesized by standard methods as known by one skilled in the art.

Alternatively, compounds of formula X, which are available from a Claisen condensation of a methyl keone and an ester using basic conditions using such bases as sodium hydride or potassium tert-butoxide, may yield compounds of formula IX via condensation and subsequent cyclization using hydroxylamine, for example in the form of the hydrochloric acid salt, at elevated temperatures (60-120° C.).

It is understood that for both methods subsequent functional group transformations may be necessary. In the case of an ester group, these transformations may include, but is not limited to either of following three procedures: a) Complete reduction using a suitable reducing agent such as LAH in solvents such as THF. b) Partial reduction using a suitable selective reducing agent such as DIBAL followed by alkylation with an alkylhalide. c) Alkylation using an alkylmetal reagent such as an alkyl magnesium halide in solvents such as toluene or THF, followed by reduction with for example sodium borohydride in methanol.
Formation of compounds of formula XIV

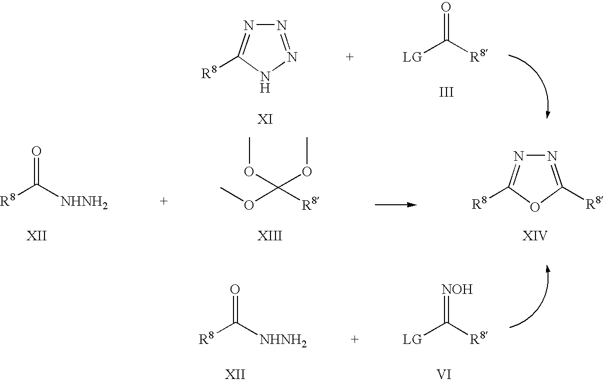

A compound of formula XIV, wherein R⁸ and R⁸′ are independently selected from a group consisting of M¹-(R²)ₙ—P—(R¹)ₘ or M² (R³)ₙ-Q(R⁴)ₘ-R⁵ or M² (R³)ₙLG² or a chemical functional group which may subsequently be transformed into M² (R³)ₙ-Q(R⁴)ₘ-R⁵, may be prepared from tetrazole compounds of type XI via acylation using an isolable compound of type III such as an acid chloride or anhydride, or a compound of type III wherein the LG may be formed in situ, for example from activation of an acid using a reagent such as DCC or EDCI, followed by rearrangement to the 1,3,4-oxadizaole. Jursic, B. S.; Zdravkovski, Z.; Synth.Commun.; (1994) 24; 1575-1582.

Alternatively, compounds of formula XIV may also be prepared from acyl hydrazide of type XII via heating in the presence of compounds of formula XIII or VI, wherein LG is a leaving group such as chloride or alkoxide, at elevated temperatures (60-130° C.) in one step. The reaction of compounds of Formula XIII may be carried out neat or using a suitable aprotic solvent such as benzene or xylene, or a protic solvent such as ethanol or n-butanol, and may be facilitated by the presence of a mild base such as KOtBu or a mild acid such as p-toluene sulfonic acid or acetic acid. Se references: Saunders, J.; Cassidy, M.; Freedman, S. B.; Harley, E. A.; Iversen, L. L. J. Med. Chem.; (1990) 33; 1128-1138; Peet, N. P.; Sunder, S. J. Heterocycl. Chem.; (1984) 21; 1807-1816. For compounds of formula VI a dehydrating agent such as phosphorous pentoxide may be used to increase cyclization of the formed reaction intermediate as has been previously been described for example by Kakefuda, Akio; et al.; Bioorg. Med. Chem. (2002), 10; 1905-1912.
Formation of Compounds of Formula XVI

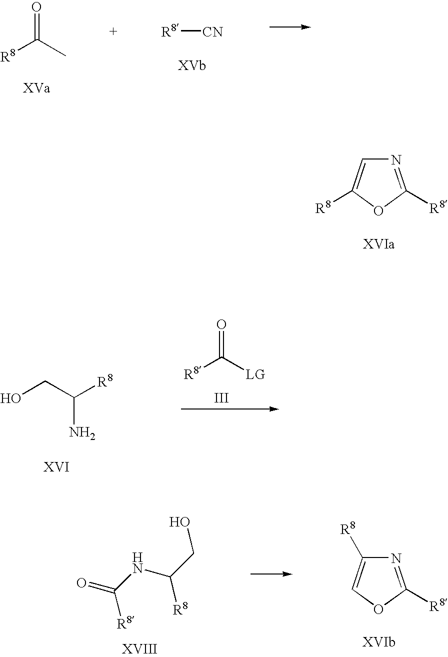

A compound of formula XVIa, wherein R⁸ and R⁸′ are independently selected from a group consisting of M¹-(R²)ₙ—P—(R¹)ₘ or M²(R³)ₙ-Q(R⁴)ₘ—R⁵ or M²(R³)ₙLG² or a chemical functional group which may subsequently be transformed into M² (R³)ₙ-Q(R⁴)ₘ—R⁵, may be prepared by the reaction of compounds of formula XVa and XVb in the presence of in situ generated Tl(OTf)3 under acidic conditions according to the procedure of Lee and Hong; Tetrahedron Lett., (1997), 38, 8959-60.

Alternatively isomer XVIb is available from reaction of compounds of formula III and XVII are reacted as described above for formula V to give an intermediate of formula XVIII. Such an intermediate may give the required oxazole by cyclodehydration with Deoxo-Fluor to generate the oxazoline followed by dehydrogenation using BrCCl₃ in the same reaction pot. Phillips, A. J.; Uto, Y.; Wipf, P.; Reno, M. J. and Williams, D. R., Organic Letters, (2000) 2, 1165-8.

General Syntheses of Compounds of Formula I

Compounds of formula I, wherein one of R⁸ and R^{8′} is M¹-(R²)ₙ—P—(R¹)ₘ and one of R⁸ and R^{8′} is M²(R³)ₙ-Q(R⁴)ₘ—R⁵, may lead directly to compounds of formula I using the general syntheses of compounds of formula V, IX, XIV or XVIa,b. For example, oxadiazoles may be formed when compound II contains M²(R³)ₙ-Q(R⁴)ₘ—R⁵, and compound III contains M¹-(R²)ₙ—P—(R¹)ₘ. In another example, isoxazoles may be formed from compounds of formula VII containing M¹-(R²)ₙ—P—(R¹)ₘ and compounds of formula VII containing M²(R³)ₙ-Q(R⁴)ₘ—R⁵.

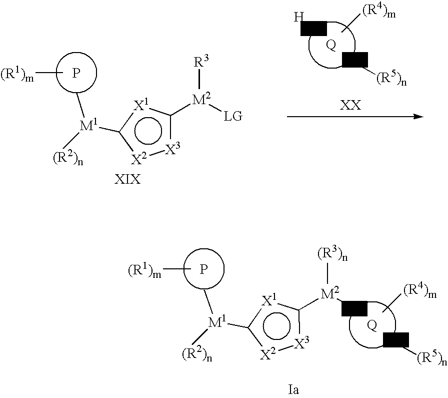

Compounds of formula XIX may be available from direct cyclization with an intermediate containing the M²(R³)LG group as described in the general syntheses of compounds of formula V, IX, XIV or XVIa,b, or may be formed subsequent to cyclization from another functional group using transformations known to one skilled in the art. For example, when an ester functional group is present, it may be reduced to the alcohol or aldehyde, which may undergo nucleophilic additions with reagents such as R³MgX to form secondary alcohols. Grignard reagents, R³MgX, when used in excess, may be added to the ester to provide the tertiary alcohol, or may provide a ketone when used in limiting quantities. The ketones and aldehydes may undergo reduction using a reducing agent such as NaBH₄ or the like, and the resulting alcohols may be converted to leaving groups, for example mesylate or chloride.

Compounds of formulae I, wherein X⁴ is N, may also be prepared from the reaction of compounds of formula XIX with an appropriate cyclic amine nucleophile of formula XX in a suitable solvent such as DMF or acetonitrile. Optional addition of an appropriate base such as potassium carbonate to absorb any excess acid produced in the reaction minimizes the equivalents of the nucleophile required. Examples of this reaction include the use of cyclic bisamines, wherein X⁵ is N, such as piperazine and homopiperazine, including N-mono-substituted piperazines which may be commercially available or may be prepared using methods known to one skilled in the art.

Monoprotected bisamines, such as N-Boc-piperazine, may lead to compounds of formula Ia, wherein X⁴ is N and R⁵ is N-Boc, and can be used to increase the scope and diversity in the R⁵ group beyond commercially available bisamines. Secondary amines of formula Ia, such as piperazines, wherein X⁴ is N and R⁵ is H, available from deprotection of such protected derivatives, are also available via reaction of the unprotected bisamine and XX, wherein X⁴ is N and R⁵═H, with the compounds of formula XIX. The secondary amine thus formed can be employed as nucleophiles in reactions with many types of electrophiles, such as alkyl halides, acid chlorides or anhydrides, chloroformates, carbamoyl chlorides, sulfonyl chlorides, isocyanates, isothiocyanates and the like.

Compounds of formulae I, wherein X⁴ is C, may be prepared from the reaction of compounds of formula VIII with an appropriate stabilized carbon nucleophile XX generated for example, using an appropriate cyclic 1,3-diketone or dithiane or the like, or where compatible, from an appropriate organometallic reagent such as an organocopper or zinc with an appropriate metal catalyst, or with an organocuprate reagent using conditions known to one skilled in the art.

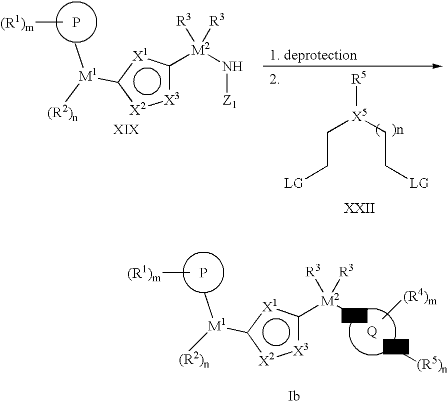

Compound of formula XXI, bearing one or more substituents R³ in the M² group, may be available from the general syntheses listed above for compounds V, IX, XIV or XVIa,b using with the appropriate starting material containing an amine residue with a suitable protecting group Z¹. For example, compounds of formula XXI wherein X¹ and X² are N and X³ is O are available from the amino acid, and as such are easily available optically enriched. Similarly, compounds of formula XXI wherein X¹ and X³ are N and X² is O are available from the amino nitrile obtained via dehydration of the primary amide formed from the acid functionality, then hydroxyamidine formation from the resulting nitrile, followed by ester formation and cyclization as above to yield the required protected aminomethyl oxadiazole of formula XXI. Isoxazoles of formula XXI wherein X¹ is C, X² is O and X³ is N may be available from compounds of formula IV via the suitably protected amino aldehyde.

The Q ring may be constructed following deprotection of the amine functionality to give compounds of formula Ib via any compatible method. One such method involves sequential displacement of the leaving groups of compound of formula XXII, wherein R⁵ is any suitable non-reactive functional group including carbamates or sulfonamides and may also be a recognized protecting group such as Boc or 2-nitrobenzene sulfonyl and LG is any suitably activated leaving group such as triflate, mesylate or chloride. It may be advantageous to use the 2-nitrobenzene sulfonyl protecting group since this may facilitate the reaction as well as the product isolation.

This method to form the piperazine ring may be employed with any methods general syntheses listed above for compounds V, IX, XIV or XVIa,b where the analogous primary amine, may be formed via displacement of LG² with ammonia, for example as a concentrated ammonium hydroxide or ammonia solution in a solvent such as methanol or dioxane, or an equivalent species such as azide which may be converted into a primary amine using conditions known to one skilled in the art.

### EXAMPLES

Embodiments of the present invention will now be illustrated by the following non-limiting examples.

NMR measurements were made on the delta scale (δ).

### Example 1

### N,N-Bis-(2-trifluoromethanesolfonyl-ethyl)-2-nitrobenzenesulfonamide

To a solution of diethanolamine (5.0 g, 47.6 mmol) in 2 N Na₂CO₃ (25 mL) at 75° C. was added nosyl chloride (10.5 g, 47.6 mmol) and the resulting mixture was heated to 95° C. for 90 min. The mixture was then cooled to room temperature and extracted with dichloromethane (3×50 mL). The organic extract was washed with brine and dried over magnesium sulfate (anhydrous) and the solvent was removed in vacuo to give the 6.2 g (45%) crude product as a yellow oil. ¹H-NMR (CDCl₃), δ (ppm): 7.95 (m, 1H), 7.70 (m, 2H), 7.61 (m, 1H), 4.04 (br, 2H), 3.82 (br, 4H), 3.46 (t, 3H).

To a solution of N,N-bis-(2-hydroxy-ethyl)-2-nitrobenzenesulfonamide (1.0 g, 3.4 mmol) in dichloromethane (20 mL) at 0° C. was added collidine (1.65 g, 13.6 mmol) followed by triflic anhydride (2.11 g, 7.5 mmol). The resulting mixture was stirred at room temperature for 2 h. The mixture was diluted with dichloromethane, washed with water, then 1 N HCl (3×20 mL). The organic extract was washed with brine and dried over magnesium sulfate (anhydrous) and the solvent was removed in vacuo to give 842 mg (48%) of the crude title compound as a white semi solid.

### Example 2

### (Cyano-methyl-methyl)-carbamic acid tert-butyl ester

A solution of N-Boc alanine (5.0 g, 26.4 mmol) in tetrahydrofuran (70 mL) was cooled to 0° C. and triethylamine (5.0 mL) was added followed by ethyl chloroformate (2.78 mL, 29.0 mmol). The resulting mixture was left stirring at room temperature for 1 h. Concentrated aqueous ammonia (11.3 mL) was added to the above reaction mixture and the clear reaction mixture was stirred at room temperature overnight. The reaction mixture was concentrated in vacuo and the isolated residue was dissolved in ethyl acetate (300 mL). The organic phase was successively washed with water (300 mL) and brine (200 mL), dried (sodium sulfate), filtered and concentrated in vacuo. Product was isolated as a white solid (2.1 g, 42%). ¹H-NMR (CDCl₃), δ (ppm): 6.20 (bs, 1H), 5.53 (bs, 1H), 5.02 (bs, 1H), 4.19 (bs, 1H), 1.42 (s, 9H), 1.24 (d, 3H).

Oxalyl chloride (7 mL, 14 mmol, 2 M dichloromethane) was added to a solution of acetonitrile (20 mL) and dimethylformamide (1.1 mL, 14 mmol) cooled to 0° C. and the resulting mixture was stirred for 15 min. This was followed by addition of a solution of (1-carbamoyl-ethyl)-carbamic acid tert-butyl ester (2.1 g, 11.2 mmol) in acetonitrile (10 mL) and pyridine (0.91 mL, 11.2 mmol). Reaction mixture was left stirring at room temperature 30 min. The reaction mixture was concentrated in vacuo and the residue was dissolved in ethyl acetate (300 mL). The organic phase was successively washed with water (300 mL) and brine (200 mL), dried (sodium sulfate), filtered and concentrated in vacuo to isolate the title compound as a white solid (1.15 g, 60%). ¹H-NMR (CDCl₃), δ (ppm): 5.05 (br, d, 1H), 4.62 (m, 1H), 1.51 (d, 3H), 1.41 (s, 9H).

### Example 3

### 2-Chloro-N-hydroxy-acetamidine

Using a modification of the procedure of Shine et al., J. Heterocyclic Chem. (1989) 26:125-128, a solution of chloroacetonitrile (20 g, 265 mmol), hydroxylamine hydrochloride (18.4 g, 265 mmol) and water (66 mL) were cooled to 15° C. using a cold water bath. Sodium carbonate (14 g, 132 mmol) was added portion-wise to the reaction mixture, keeping the temperature below 30° C. The reaction mixture was stirred at 30° C. for 1 h using a warm water bath. Solid sodium chloride was added to the reaction mixture. The aqueous phase was extracted with diethyl ether (4×150 mL). Combined organic phase was dried (sodium sulfate), filtered and concentrated in vacuo. Crude residue was triturated with a mixture of diethyl ether in hexanes to isolate the title compound (13.5 g) as a lemon yellow solid. ¹H-NMR (CDCl₃), δ (ppm): 4.71 (bs, 2H), 4.04 (s, 2H).

### Example 4

### [1-(N-Hydroxycarbamimidoyl)-ethyl]-1-carbamic acid tert-butyl ester

[1-(N-Hydroxycarbamimidoyl)-ethyl]-1-carbamic acid tert-butyl ester (1.01 g, 74%, white solid) was prepared as described for example 3 using hydroxylamine hydrochloride (2.35 g, 33.8 mmol), sodium carbonate (3.58 g, 33.8 mmol) in water (50 mL), methyl alcohol (50 mL) and (cyano-methyl-methyl)-carbamic acid tert-butyl ester (1.15 g, 6.76 mmol). The product was used without further purification.

### Example 5

### 3-Chloromethyl-5-m-tolyl-[1,2,4]oxadiazole

3-Methyl-benzoyl chloride (802 μL, 6.1 mmol) was added to a suspension of 2-chloro-N-hydroxy-acetamidine (440 mg, 4.1 mmol) in dichloromethane (10 mL) at room temperature. After stirring for 30 min., triethylamine (622 μL, 4.5 mmol) was added and stirred for an additional hour. The reaction mixture was diluted with dichloromethane, washed with water and brine, dried over anhydrous sodium sulfate, filtered and concentrated in vacuo. Flash column chromatography using 10-20% ethyl acetate in hexanes afforded 814 mg of the acyclic ester intermediate. DMF was added to this intermediate and then heated at 135° C. for 4 h to effect cyclization to oxadiazole. After cooling the reaction mixture was washed with water (3 times) and brine, dried over anhydrous sodium sulfate, filtered, and concentrated. Purification by flash column chromatography on silica gel using 5% ethyl acetate in hexanes afforded 3-chloromethyl-5-m-tolyl-[1,2,4]oxadiazole, 469 mg (54% over 2 steps) as a white solid. ¹H NMR (CDCl₃), δ (ppm): 7.99 (s, 1H), 7.97 (m, 1H), 7.43 (d, 2H), 4.68 (s, 2H), 2.45 (s, 3H).

### Example 6

### 3-(3-Chloromethyl-[1,2,4]oxadiazol-5-yl)-benzonitrile

3-(3-Chloromethyl-[1,2,4]oxadiazol-5-yl)-benzonitrile (3.57 g, 43%) was prepared as described for example 5 using 2-chloro-N-hydroxy-acetamidine (4.05 g, 37.4 mmol) and 3-cyanobenzoyl-chloride (6.2 g, 37.4 mmol) in dichloromethane (60 mL) with triethylamine (6.5 mL, 46.7 mmol). Purification was performed by silica gel chromatography. ¹H NMR (CDCl₃), δ (ppm): 8.47 (bs, 1H), 8.41 (dd, 1H), 7.91 (dd, 1H), 7.72(t, 1H), 4.70 (s, 2H); GC-MS (M+): 219.

### Example 7

### 3-Chloromethyl-5-(3-fluoro-phenyl)-[1,2,4]oxadiazole

DMF (10 mL) was added to a mixture of 3-fluorobenzoic acid (710 mg, 5.07 mmol), EDCI (972 mg, 5.07 mmol), HOBt (685 mg, 5.07 mmol) and 2-chloro-N-hydroxy-acetamidine (500 mg, 4.61 mmol) at room temperature and then stirred overnight. The reaction mixture was diluted with ethyl acetate, washed with water (3 times) and brine, dried over anhydrous sodium sulfate, filtered and concentrated. DMF (14 mL) was added to the residue and the resulting solution was heated 135° C. for 3.5 h to effect cyclization to oxadiazole. After cooling the reaction mixture was washed with water (3 times) and brine, dried over anhydrous sodium sulfate, filtered, and concentrated. 3-Chloromethyl-5-(3-fluoro-phenyl)-[1,2,4]oxadiazole (383 mg, 35% yield over 2 steps, yellow oil) was obtained by flash chromatography on silica gel, using 5% ethyl acetate in hexane. ¹H NMR (CDCl₃) δ (ppm): 7.96 (d, 1H), 7.86 (m, 1H), 7.54 (m, 1H), 7.33 (m, 1H), 4.68 (s, 2H).

Examples 8 to 12 were prepared as described for example 7.

### Example 8

### 3-Chloromethyl-5-(3-iodo-phenyl)-[1,2,4]oxadiazole

3-Chloromethyl-5-(3-iodo-phenyl)-[1,2,4]oxadiazole (2.9 g, 44%, white solid) was obtained from 3-iodo-benzoic acid (5.0 g, 20.2 mmol), 2-chloro-N-hydroxy-acetamidine (2.4 g, 22.2 mmol), EDCI (4.3 g, 22.2 mmol) and HOBt (3.0 g, 22.2 mmol) in DMF (10 mL). The acyclic ester intermediate was purified by flash column chromatography using 50-80% ethyl acetate in hexanes. Purification of the title compound was performed by SPE (flash) chromatography using 5% ethyl acetate in hexanes. ¹H NMR (CDCl₃), δ (ppm): 8.52 (s, 1H), 8.13 (d, 1H), 7.96 (d, 1H), 7.29 (t, 1H), 4.68 (s, 2H).

### Example 9

### 3-Chloromethyl-5-(3-chloro-phenyl)-[1,2,4]oxadiazole

3-Chloromethyl-5-(3-chloro-phenyl)-[1,2,4]oxadiazole (406 mg, 43% yield over 2 steps, white solid) was obtained from 3-chlorobenzoic acid (708 mg, 4.52 mmol), EDCI (866 mg, 4.52 mmol), HOBt (611 mg, 4.52 mmol) and 2-chloro-N-hydroxy-acetamidine (446 mg, 4.11 mmol) in DMF (10 mL). Purification was performed by flash column chromatography using 5% ethyl acetate in hexane. ¹H NMR (CDCl₃) δ (ppm): 8.17 (t, 1H), 8.05 (d, 1H), 7.59 (t, 1H), 7.50 (t, 1H), 4.68 (s, 2H)

### Example 10

### 3-Chloromethyl-5-(3-trifluoromethoxy-phenyl)-[1,2,4]oxadiazole

3-Chloromethyl-5-(3-trifluoromethoxy-phenyl)-[1,2,4]oxadiazole (707 mg, 55% yield over 2 steps, light yellow oil) was obtained from 3-trifluoromethoxybenzoic acid (1.05 g, 5.07 mmol), EDCI (972 mg, 5.07 mmol), HOBt (685 mg, 5.07 mmol) and 2-chloro-N-hydroxy-acetamidine (500 mg, 4.61 mmol) in DMF (10 mL). Purification was performed by flash column chromatography using 5% ethyl acetate in hexane. ¹H NMR (CDCl₃) δ (ppm): 8.10 (m, 1H), 8.03 (s, 1H), 7.61 (t, 1H), 7.48 (d, 1H), 4.69 (s, 2H)

### Example 11

### 5-(3-Bromo-phenyl)-3-chloromethyl-[1,2,4]oxadiazole

5-(3-Bromo-phenyl)-3-chloromethyl-[1,2,4]oxadiazole (707 mg, 55% yield over 2 steps, white solid) was obtained from 3-bromobenzoic acid (1.05 g, 5.07 mmol), EDCI (972 mg, 5.07 mmol), HOBt (685 mg, 5.07 mmol) and 2-chloro-N-hydroxy-acetamidine (500 mg, 4.61 mmol) in DMF (10 mL). Purification was performed by flash column chromatography using 5% ethyl acetate in hexane. ¹H NMR (CDCl₃) δ (ppm): 8.10 (m, 1H), 8.03 (s, 1H), 7.61 (t, 1H), 7.48 (d, 1H), 4.69 (s, 2H)

### Example 12

### 1-(5-(3-Methylphenyl-[1,2,4]oxadiazol-3-yl)-ethylamine

[1-5-(3-Methylphenyl)-[1,2,4]oxadiazol-3-yl)-ethyl]-carbamic acid tert-butyl ester was obtained from [1-(N-hydroxycarbamimidoyl)-ethyl]-1-carbamic acid tert-butyl ester (Example 4) (1.01 g, 4.97 mmol), m-toluic acid (680 mg, 5.0 mmol) and EDCI (959 mg, 5.0 mmol), HOBt (675 mg, 5.0 mmol), DMF (15 mL). The crude residue was deprotected without further purification.

Trifluoroacetic acid (5 mL) was added to a solution of [1-5-(3-methylphenyl)-[1,2,4]oxadiazol-3-yl)-ethyl]-carbamic acid tert-butyl ester in dichloromethane (5 mL) at 0° C. The resulting mixture was stirred at this temperature for 90 min., and then added to cold saturated NaHCO₃ and the resulting neutralized mixture was extracted with dichloromethane (30 mL). The organic extract was washed with brine and dried over magnesium sulfate (anhydrous) and the solvent was removed in vacuo. The residue was then purified by flash column silica gel chromatography with 5% (2 M ammonia methanol) in dichloromethane as eluant giving 280 mg (79%) of the title compound as a light brown oil. ¹H-NMR (CDCl₃), δ (ppm): 7.92 (m, 2H), 7.40 (m, 2H), 4.26 (q, 1H), 2.43 (s, 3H), 1.76 (br, 2H), 1.55 (d, 3H).

### Example 13

### 1-[1-(5-(3-Methyl-phenyl)-[1,2,4]oxadiazol-3-yl)-ethyl]-piperazine

To a solution of 1-(5-(3-methyphenylyl-[1,2,4]oxadiazol-3-yl)-ethylamine (270 mg, 1.33 mmol) and N,N-Bis-(2-trifluoromethanesolfonyl-ethyl)-2-nitrobenzenesulfonamide (842 mg, 1.52 mmol) in acetonitrile (25 mL) was added Na₂CO₃ (282 mg, 2.66 mmol) and the mixture was stirred vigorously at room temperature for 24 h. The mixture was diluted with ethyl acetate and washed with water. The organic extract was then washed with brine and dried over magnesium sulfate (anhydrous) and the solvent was removed in vacuo. The residue was then purified by flash column silica gel chromatography with 5% (2 M ammonia methanol) in dichloromethane as eluant giving 101 mg (84%) of the product as a yellow oil. ¹H-NMR (CDCl₃), δ (ppm): ): 7.96 (m, 3H), 7.70 (m, 2H), 7.55 (m, 1H), 7.40 (m, 2H), 4.10 (q, 1H), 3.38 (t, 4H), 2.70 (t, 4H), 2.45 (s, 3H), 1.55 (d, 3H).

To a solution of 1-(2-nitrobenzenesulfonyl)-4-[1-(5-(3-methyl-phenyl)-[1,2,4]oxadiazol-3-yl)-ethyl]-piperazine (501 mg, 1.10 mmol) in DMF (10 mL) was added LiOH (189 mg, 4.4 mmol) followed by mercaptoacetic acid (202 mg, 2.2 mmol) and the mixture was stirred at room temperature for 90 min. The mixture was diluted with dichloromethane and washed with water. The organic extract was then washed with brine and dried over magnesium sulfate (anhydrous) and the solvent was removed in vacuo. The residue was purified by flash column silica gel chromatography with ethylacetate/hexane as eluant giving 101 mg (34%) of the title compound as a yellow oil. ¹H-NMR (CDCl₃), δ (ppm): 7.96 (m, 2H), 7.40 (m, 2H), 3.98 (q, 1H), 2.97 (t, 4H), 2.60 (t, 4H), 2.42 (s, 3H), 1.80 (br, 1H), 1.45 (d, 3H).

### Example 14

### 4-(5-m-Tolyl-[1,2,4]oxadiazol-3-ylmethyl)-piperazine-1-carboxylic acid ethyl ester hydrochloride

Piperazine-1-carboxylic acid ethyl ester (42 μL, 0.29 mmol) was added to a mixture of 3-chloromethyl-5-m-tolyl-[1,2,4]oxadiazole (50 mg, 0.24 mmol) and potassium carbonate (99 mg, 0.72 mmol) in acetonitrile (1 mL) and the resulting mixture was stirred at room temperature overnight. The reaction mixture was diluted with ethyl acetate, washed with water and brine, dried over anhydrous sodium sulfate, filtered and concentrated. The title compound was obtained by solid phase extraction chromatography (SPE) on silica gel using 10-50% ethyl acetate in hexanes. ¹H NMR (CDCl₃), δ (ppm): 7.98 (s, 1H), 7.94 (m, 1H), 7.40 (d, 2H), 4.12 (q, 2H), 3.78 (s, 2H), 3.54 (t, 4H), 2.58 (t, 4H), 2.43 (s, 3H), 1.24 (t, 3H).

1 M HCl in diethyl ether (1.2 mL) was added to a solution of 4-(5-m-tolyl-[1,2,4]oxadiazol-3-ylmethyl)-piperazine-1-carboxylic acid ethyl ester (97 mg, 0.29 mmol) in dichloromethane (2 mL) at 0° C. and then warmed to room temperature. After stirring for 30 min., the reaction mixture was diluted with diethyl ether and then sonicated. The precipitate was isolated by filtration to afford the title compound, 74 mg (70%) as a white solid. ¹H NMR (DMSO), δ (ppm): 7.97 (m, 2H), 7.57 (m, 2H), 4.54 (bs, 2H), 4.06 (q, 2H), 3.45 (bs, 8H), 2.43 (s, 3H), 1.19 (t, 3H). LS-MS (ES+full scan, C₁₇H₂₂N₄O₃) M⁺ calc. 330.17, found (M+1)⁺ 331.17.

Examples 15 to 24 were prepared as described for example 14, with the optional salt formation from the free base generated.

### Example 15

### 4-[5-(3-Methoxyphenyl)-[1,2,4]oxadiazol-3-ylmethyl)-piperazine-1-carboxylic acid ethyl ester hydrochloride

4-[5-(3-Methoxyphenyl)-[1,2,4]oxadiazol-3-ylmethyl)-piperazine-1-carboxylic acid ethyl ester hydrochloride (14 mg, white solid) was obtained from piperazine-1-carboxylic acid ethyl ester (108 mg, 0.68 mmol), 3-chloromethyl-5-(3-methoxy-phenyl)-[1,2,4]oxadiazole (30 mg, 0.13 mmol), K₂CO₃ (50 mg, 0.36 mmol) in acetonitrile (2 mL) at 80°for 2 h. Purification was performed by silica gel chromatography. The oil was converted to HCl salt as described for Example 14. ¹H-NMR (CD₃OD), δ (ppm): 7.76 (d, 1H), 7.70 (s, 1H), 7.53 (t, 1H), 7.27 (d, 1H), 4.84 (m, 4H), 4.73 (s, 2H), 4.16 (q, 2H), 3.88 (s, 3H), 3.51 (m, 4H), 1.27 (t, 3H).

### Example 16

### 1-(5-m-Tolyl-[1,2,4]oxadiazol-3-ylmethyl)-piperazine

1-(5-m-Tolyl-[1,2,4]oxadiazol-3-ylmethyl)-piperazine (598 mg, 97%, white waxy solid) was obtained from piperazine (1.45 g, 16.8 mmol) in tetrahydrofuran (15 mL) and 3-chloromethyl-5-m-tolyl-[1,2,4,]oxadiazole (500 mg, 2.40 mmol) in tetrahydrofuran (5 mL) (note: reverse order of addition). Purification was performed on silica gel using 10% ammonia (2 N methanol) in dichloromethane. ¹H-NMR (CDCl₃), δ (ppm): 7.95 (m, 2H), 7.39 (m, 2H), 3.75 (s, 2H), 2.96 (m, 4H), 2.61 (m, 4H), 2.43 (s, 3H), 2.00 (bs, 1H).

### Example 17

### 1-[5-(3-Methoxy-phenyl)-[1,2,4]oxadiazol-3-ylmethyl]-3-methyl-piperazine

1-[5-(3-Methoxy-phenyl)-[1,2,4]oxadiazol-3-ylmethyl]-3-methyl-piperazine (124.4 mg, 97%, colorless oil) was obtained from 3-chloromethyl-5-(3-methoxy-phenyl)-[1,2,4]oxadiazole (100 mg, 0.444 mmol), potassium carbonate (156.3 mg, 1.112 mmol), and (±)-2-methylpiperazine (111.5 mg, 1.112 mmol) in acetonitrile (3 mL). Purification by SPE flash chromatography using 7% 2 M ammonia in methanol in dichloromethane yielded a colorless oil.

### Example 18

### 4-[5-(3-Trifluoromethyl-phenyl)-[1,2,4]oxadiazol-3-ylmethyl]-piperazine-1-carboxylic acid ethyl ester

4-[5-(3-Trifluoromethyl-phenyl)-[1,2,4]oxadiazol-3-ylmethyl]-piperazine-1-carboxylic acid ethyl ester (19 mg, 21%, colorless oil) was obtained from 3-chloromethyl-5-(3-trifluoromethyl-phenyl)-[1,2,4]oxadiazole (60 mg, 0.23 mmol), potassium carbonate (95 mg, 0.69 mmol), and piperazine-1-carboxylic acid ethyl ester (40 μL, 0.27 mmol) in acetonitrile (1 mL). Purification was performed by SPE (flash) chromatography using 15-40% ethyl acetate in hexanes. ¹H NMR (CDCl₃), δ (ppm): 8.46 (s, 1H), 8.35 (d, 1H), 7.87 (d, 1H), 7.70 (t, 1H), 4.14 (q, 2H), 3.81 (s, 2H), 3.56 (t, 4H), 2.60 (t, 4H), 1.26 (t, 3H).

### Example 19

### 4-[5-(3-Cyano-phenyl)-[1,2,4]oxadiazol-3-ylmethyl]-piperazine-1-carboxylic acid ethyl ester)

4-[5-(3-Cyano-phenyl)-[1,2,4]oxadiazol-3-ylmethyl]-piperazine-1-carboxylic acid ethyl ester (194 mg, 64%) was obtained from 3-(3-chloromethyl-[1,2,4]oxadiazol-5-yl)-benzonitrile (200 mg, 0.91 mmol) and piperazine-1-carboxylic acid ethyl ester (0.16 mL, 1.09 mmol) in acetonitrile with K₂CO₃ (0.378 g, 2.73 mmol). Purification was performed by silica gel chromatography using 50% ethyl acetate in dichloromethane. ¹H NMR (CDCl₃), δ (ppm): 8.47(t, 1H), 8.39(d, 1H), 7.89(d, 1H), 7.70(t, 1H), 4.13(q, 2H), 3.81(s, 1H), 3.55(t, 4H), 2.60(t, 4H), 1.26(t, 3H); LC-MS (M+H)⁺: 342.

### Example 20

### 4-[5-(3-Fluoro-phenyl)-[1,2,4]oxadiazol-3-ylmethyl]-piperazine-1-carboxylic acid ethyl ester

4-[5-(3-Fluoro-phenyl)-[1,2,4]oxadiazol-3-ylmethyl]-piperazine-1-carboxylic acid ethyl ester (43.1 mg, 54%, colorless oil) was obtained from piperazine-1-carboxylic acid ethyl ester (39 mg, 0.25 mmol), 3-chloromethyl-5-(3-fluoro-phenyl)-[1,2,4]oxadiazole (50 mg, 0.24 mmol) and potassium carbonate (98 mg, 0.71 mmol) in acetonitrile (1 mL). Purification was performed by SPE (flash) chromatography using 40-50% ethyl acetate in hexane. ¹H NMR (CDCl₃) δ (ppm): 7.96 (d, 1H), 7.86 (t, 1H), 7.52 (m, 1H), 7.31 (m, 1H), 4.13 (m, 2H), 3.79 (s, 2H), 3.55 (t, 4H), 2.60 (t, 1H), 1.26 (t, 3H)

### Example 21

### 4-[5-(3-Iodo-phenyl)-[1,2,4]oxadiazol-3-ylmethyl-piperazine-1-carboxylic acid ethyl ester

4-[5-(3-Iodo-phenyl)-[1,2,4]oxadiazol-3-ylmethyl]-piperazine-1-carboxylic acid ethyl ester (568 mg, 82%, white solid) was obtained from 3-chloromethyl-5-(3-iodo-phenyl)-[1,2,4]oxadiazole (500 mg, 1.56 mmol), potassium carbonate (647 mg, 4.68 mmol), and piperazine-1-carboxylic acid ethyl ester (457 μL, 3.12 mmol) in acetonitrile (10 mL). Purification was performed by flash column chromatography on silica gel using 20-40% ethyl acetate in hexanes. ¹H NMR (CDCl₃), δ (ppm): 8.54 (s, 1H), 8.12 (d, 1H), 7.93 (d, 1H), 7.28 (t, 1H), 4.13 (q, 2H), 3.78 (s, 2H), 3.55 (t, 4H), 2.59 (t, 4H), 1.26 (t, 3H).

### Example 22

### 4-[5-(3-Chloro-phenyl)-[1,2,4]oxadiazol-3-ylmethyl]-piperazine-1-carboxylic acid ethyl ester

4-[5-(3-Chloro-phenyl)-[1,2,4]oxadiazol-3-ylmethyl]-piperazine-1-carboxylic acid ethyl ester (56.1 mg, 66%, white solid) was obtained from piperazine-1-carboxylic acid ethyl ester (66 mg, 0.42 mmol), 3-chloromethyl-5-(3-chloro-phenyl)-[1,2,4]oxadiazole (50 mg, 0.22 mmol) and potassium carbonate (91 mg, 0.66 mmol) in acetonitrile (1 mL). Purification was performed by SPE (flash) chromatography using 45% ethyl acetate in hexane. ¹H NMR (CDCL₃) δ (ppm): 8.18 (t, 1H), 8.04 (t, 1H), 7.57 (t, 1H), 7.48 (t, 1H), 4.13 (m, 2H), 3.79 (s, 2H), 3.55 (t, 4H), 2.59 (t, 4H), 1.26 (t, 3H).

### Example 23

### 4-[5-(3-Trifluoromethoxy-phenyl)-[1,2,4]oxadiazol-3-ylmethyl]-piperazine-1-carboxylic acid ethyl ester

4-[5-(3-Trifluoromethoxy-phenyl)-[1,2,4]oxadiazol-3-ylmethyl]-piperazine-1-carboxylic acid ethyl ester (153 mg, 100%, white solid) was obtained from piperazine-1-carboxylic acid ethyl ester (108 mg, 0.68 mmol), 3-chloromethyl-5-(3-trifluoromethoxy-phenyl)-[1,2,4]oxadiazole (100 mg, 0.36 mmol) and potassium carbonate (149 mg, 1.08 mmol) in acetonitrile (2 mL). Purification was performed by SPE (flash) chromatography using 40% ethyl acetate in hexane. ¹H NMR (CDCL₃) δ (ppm): 8.11 (d, 1H), 8.03 (s, 1H), 7.59 (t, 1H), 7.46 (d, 1H), 4.13 (m, 2H), 3.80 (m, 2H), 3.55 (t, 4H), 2.60 (t, 4H), 1.26 (t, 3H)

### Example 24

### 4-[5-(3-Bromo-phenyl)-[1,2,4]oxadiazol-3-ylmethyl]-piperazine-1-carboxylic acid ethyl ester

4-[5-(3-Bromo-phenyl)-[1,2,4]oxadiazol-3-ylmethyl]-piperazine-1-carboxylic acid ethyl ester (65.4 mg, 75%, white solid) was obtained from piperazine-1-carboxylic acid ethyl ester (66 mg, 0.42 mmol), 5-(3-bromo-phenyl)-3-chloromethyl-[1,2,4]oxadiazole (60 mg, 0.22 mmol), and potassium carbonate (91 mg, 0.66 mmol) in acetonitrile (2 mL). Purification was performed by SPE (flash) chromatography using 40% ethyl acetate in hexane. ¹H NMR (CDCL₃) δ (ppm): 8.33 (s, 1H), 8.09 (d, 1H), 7.73, (d, 1H), 7.42 (t, 1H), 4.13 (m, 2H), 3.79 (s, 2H), 3.55 (t, 4H), 2.59 (t, 4H), 1.26 (t, 3H)

### Example 25

### 4-(5-m-Tolyl-[1,2,4]oxadiazol-3-ylmethyl)-piperazine-1-carboxylic acid methyl ester

In a screw cap vial equipped with stir bar, added 1-(5-m-tolyl-[1,2,4]oxadiazol-3-ylmethyl)-piperazine (50 mg, 0.15 mmol), dichloromethane (2 mL) and triethylamine (60 μl, 0.46 mmol). To this mixture was added methyl chloroformate (20 μl, 0.23 mmol). The reaction mixture was stirred at room temperature overnight, after which it was concentrated in vacuo and the residue was dissolved in ethyl acetate (10 mL). The organic phase was sequentially washed with water (3×10 mL), brine (10 mL), dried (sodium sulfate), filtered and concentrated in vacuo. Purification of the crude residue was performed on silica gel using 50% ethyl acetate in hexanes to isolate the title compound (40 mg, 84%) as clear oil. ¹H-NMR (CDCl₃), δ (ppm): 7.95 (m, 2H), 7.40 (m, 2H), 3.77 (s, 2H), 3.68 (s, 3H), 3.54 (m, 4H), 2.59 (m, 4H), 2.43 (s, 3H).

Examples 26 to 30 were prepared as described for example 25.

### Example 26

### 4-(5-m-Tolyl-[1,2,4]oxadiazol-3-ylmethyl)-piperazine-1-carboxylic acid propyl ester

4-(5-m-Tolyl-[1,2,4]oxadiazol-3-ylmethyl)-piperazine-1-carboxylic acid propyl ester (35.8 mg, 69%, clear oil) was obtained from 1-(5-m-tolyl-[1,2,4]oxadiazol-3-ylmethyl)-piperazine (50 mg, 0.15 mmol) and n-propyl chloroformate (30 μl, 0.23 mmol) in dichloromethane (2 mL) and triethylamine (60 μl, 0.46 mmol). Purification was performed by silica gel chromatography. ¹H-NMR (CDCl₃), δ (ppm): 7.95 (m, 2H), 7.40 (m, 2H), 4.03 (t, 2H), 3.78 (s, 2H), 3.54 (m, 4H), 2.59 (m, 4H), 2.43 (s, 3H), 1.66 (m, 2H), 0.93 (t, 3H).

### Example 27

### 4-(5-m-Tolyl-[1,2,4]oxadiazol-3-ylmethyl)-piperazine-1-carboxylic acid butyl ester

4-(5-m-Tolyl-[1,2,4]oxadiazol-3-ylmethyl)-piperazine-1-carboxylic acid butyl ester (41 mg, 76%, clear oil) was obtained from 1-(5-m-tolyl-[1,2,4]oxadiazol-3-ylmethyl)-piperazine (50 mg, 0.15 mmol) and added n-butyl chloroformate (30 μl, 0.23 mmol) in dichloromethane (2 mL) and triethylamine (60 μl, 0.46 mmol). Purification was performed by silica gel chromatography. ¹H-NMR (CDCl₃), δ (ppm): 7.95 (m, 2H), 7.40 (m, 2H), 4.07 (t, 2H), 3.78 (s, 2H), 3.54 (m, 4H), 2.59 (m, 4H), 2.43 (s, 3H), 1.61 (m, 2H), 1.34 (m, 2H), 0.92 (t, 3H).

### Example 28

### 4-[5-(3-Methoxy-phenyl)-[1,2,4]oxadiazol-3-ylmethyl]-2-methyl-piperazine-1-carboxylic acid ethyl ester

4-[5-(3-Methoxy-phenyl)-[1,2,4]oxadiazol-3-ylmethyl]-2-methyl-piperazine-1-carboxylic acid ethyl ester (100 mg, 89.2%, pinkish oil) was obtained from 1-[5-(3-methoxy-phenyl)-[1,2,4]oxadiazol-3-ylmethyl]-3-methyl-piperazine (120 mg, 0.416 mmol) with ethylchloroformate (160 μl, 0.62 mmol), triethylamine (0.29 ml, 2.08 mmol) and dichloromethane (4 mL) Purification was performed by silica gel chromatography. ¹H NMR (CDCl₃), δ (ppm): 7.73 (d, 1H), 7.64 (s, 1H), 7.43 (t, 1H), 7.13 (dd, 1H), 4.29 (m, 1H), 4.12 (t, 2H), 3.92 (m, 1H), 3.88 (s, 3H), 3.75 (dd, 2H), 3.24 (td, 1H), 2,94 (dd, 1H), 2.74 (dd, 1H), 2.37 (dd, 1H), 2.26 (td, 1H), 1.26 (t, 3H), 1.25 (d, 3H).

### Example 29

### 4-(5-m-Tolyl-[1,2,4]oxadiazol-3-ylmethyl)-piperazine-1-carboxylic acid isopropyl ester

4-(5-m-Tolyl-[1,2,4]oxadiazol-3-ylmethyl)-piperazine-1-carboxylic acid isopropyl ester (46.1 mg, 89%, clear oil) was obtained from 1-(5-m-tolyl-[1,2,4]oxadiazol-3-ylmethyl)-piperazine (50 mg, 0.15 mmol) and isopropyl chloroformate (0.23 mL, 0.23 mmol, 1 M toluene) in dichloromethane (2 mL) and triethylamine (60 μl, 0.46 mmol). Purification was performed on silica gel using 80% ethyl acetate in hexanes. ¹H-NMR (CDCl₃), δ (ppm): 7.95 (m, 2H), 7.40 (m, 2H), 4.91 (m, 1H), 3.78 (s, 2H), 3.53 (m, 4H), 2.58 (m, 4H), 2.43 (s, 3H), 1.23 (d, 6H).

### Example 30

### 4-[1-(5-(3-Methyl-phenyl)-[1,2,4]oxadiazol-3-yl)-ethyl]-piperazine-carboxylic acid ethyl ester

To a solution of 1-[1-(5-(3-methyl-phenyl)-[1,2,4]oxadiazol-3-yl)-ethyl]-piperazine (75 mg, 0.28 mmol) and Et₃N (0.4 mL, 2.88 mmol) in dichloromethane (5 mL) at 0° C. was added ethylchloroformate (60 mg, 0.55 mmol) and the mixture was stirred at room temperature overnight. The mixture was diluted with dichloromethane and washed with water. The organic extract was then washed with brine and dried over magnesium sulfate (anhydrous) and the solvent was removed in vacuo. The residue was then purified by flash column silica gel chromatography with ethylacetate/hexane as eluant giving 63 mg (65%) of the title compound as a colourless oil. ¹H-NMR (CDCl₃), δ (ppm) 7.94 (m, 2H), 7.40 (m, 2H), 4.10 (q, 1H), 4.02(q, 1H), 3.50 (t, 4H), 2.57 (t, 4H), 2.43 (s, 3H), 1.53 (d, 3H), 1.22 (t, 3H).

### Example 31

### 4-[5-(3-Furan-3-yl-phenyl)-[1,2,4]oxadiazol-3-ylmethyl]-piperazine-1-carboxylic acid ethyl ester

To 4-[5-(3-iodo-phenyl)-[1,2,4]oxadiazol-3-ylmethyl]-piperazine-1-carboxylic acid ethyl ester (50 mg, 0.11 mmol) in a vial was added 3-furan boronic acid (25 mg, 0.23 mmol), tetrakis(triphenylphosphine)palladium(0) (13 mg, 0.011 mmol), ethylene glycol dimethyl ether (1 mL) and 2 M sodium carbonate (1 mL). The vial was then sealed and heated at 90° C. for 1 h with vigorous stirring. The reaction was cooled, diluted with ethyl acetate, washed with water and saturated brine, filtered, and concentrated. The residue was purified by flash column chromatography using 40% ethyl acetate in hexanes. Additional purification by trituration with hexanes and filtration afforded the title compound as a beige solid 17 mg (38%). ¹H NMR (CDCl₃), δ (ppm): 8.28 (d, 1H), 8.05 (d, 1H), 7.84 (s, 1H), 7.72 (d, 1H), 7.53 (m, 2H), 6.79 (s, 1H), 4.14 (q, 2H), 3.81 (s, 2H), 3.56 (t, 4H), 2.60 (t, 4H), 1.26 (t, 3H).

### Example 32

### Synthesis of 3(R)-Methyl-piperazine-1-carboxylic acid ethyl ester and 3(S)-Methyl-piperazine-1-carboxylic acid ethyl ester

(R)-3-Methyl-piperazine-1-carboxylic acid ethyl ester (502 mg, 62%, a light brown oil) and (S)-3-Methyl-piperazine-1-carboxylic acid ethyl ester (307 mg, 38%, a light brown oil) was obtained from (R)-2-Methyl-piperazine (1.0 g, 9.98 mmol) or (S)-2-Methyl-piperazine (1.0 g, 9.98 mmol) and ethylchloroformate (0.45 ml, 4.71 mmol) in dichloromethane (5 mL). Purification was performed by silica gel chromatography. ¹H-NMR (CDCl₃), δ (ppm): 4.13 (q, 2H), 3.91 (m, 2H), 2.70 (m, 4H), 2.42 (m, 1H), 1.76 (br, s, 1H), 1.23 (t, 3H), 1.00 (d, 3H).

Examples 33-35 were prepared as described for example 2.

### Example 33

### (S)-(Cyano-methyl-methyl)-carbamic acid tert-butyl ester

(S)-(Cyano-methyl-methyl)-carbamic acid tert-butyl ester (8.0 g, white solid) were prepared as described in example 2 from N-Boc-L-alanine (15.0 g, 79.2 mmol).

### Example 34

### (R)-(Cyano-methyl-methyl)-carbamic acid tert-butyl ester

(R)-(Cyano-methyl-methyl)-carbamic acid tert-butyl ester (3.55 g, white solid) were prepared as described in example 2 from N-Boc-D-alanine (7.5 g, 39.6 mmol).

### Example 35

### (1-Cyano-propyl)-carbamic acid tert-butyl ester

(1-Cyano-propyl)-carbamic acid tert-butyl ester (2.55 g, white solid)was prepared as described in example 2 from 2-t-Butoxycarbonylamino-butyric acid (5 g, 24.6 mmol).

Example 36-38 were prepared as described for example 4.

### Example 36

### (S)-[1-(N-Hydroxycarbamimidoyl)-ethyl]-1-carbamic acid tert-butyl ester

The title compound (2.35 g, 86%, white solid) was prepared as described for example 3 from (S)-(cyano-methyl-methyl)-carbamic acid tert-butyl ester (2.3 g, 13.5 mmol). The product was used without further purification.

### Example 37

### (R)-[1-(N-Hydroxycarbamimidoyl)-ethyl]-1-carbamic acid tert-butyl ester

The title compound (2.92 g, 69%, white solid) was prepared as described for example 3 from (R)-(cyano-methyl-methyl)-carbamic acid tert-butyl ester (3.55 g, 20.9 mmol). The product was used without further purification.

### Example 38

### [1-(N-Hydroxycarbamimidoyl)-propyl]-carbamic acid tert-butyl ester

The title compound (2.5 g, white solid) was prepared 4 using hydroxylamine hydrochloride (4.81 g, 13.8 mmol), sodium carbonate (7.33 g, 69.2 mmol) in water (75 mL), methyl alcohol (75 mL) and (cyano-methyl-methyl)-carbamic acid tert-butyl ester (2.55 g, 13.8 mmol). The product was used without further purification.

Examples 39-44 were prepared as described for example 12.

### Example 39

### (S)-1-(5-(3-Methylphenyl-[1,2,4]oxadiazol-3-yl)-ethylamine

The title compound (226 mg, 56%, pale yellow oil) was obtained from toluic acid (340 mg, 2.5 mmol). ¹H-NMR (CDCl₃), δ (ppm): 7.92 (m, 2H), 7.40 (m, 2H), 4.26 (q, 1H), 2.43 (s, 3H), 1.76 (br, 2H), 1.55 (d, 3H).

### Example 40

### (R)-1-(5-(3-Methylphenyl-[1,2,4]oxadiazol-3-yl)-ethylamine

The title compound (203 mg, pale yellow oil) was obtained from toluic acid (915 mg, 6.77 mmol). ¹H-NMR (CDCl₃), δ (ppm): 7.92 (m, 2H), 7.40 (m, 2H), 4.26 (q, 1H), 2.43 (s, 3H), 1.76 (br, 2H), 1.55 (d, 3H).

### Example 41

### (S)-1-[5-(2-Fluoro-5-methyl-phenyl)-[1,2,4]oxadiazol-3-yl]-ethylamine

The title compound (295 mg, pale yellow oil) was obtained from 2-Fluoro-5-methyl benzoic acid (385 mg, 2.5 mmol). ¹H-NMR (CDCl₃), δ (ppm): 7.91 (dd, 1H), 7.37 (m, 1H), 7.16 (dd, 1H), 4.32 (q, 1H), 2.42 (s, 3H), 1.76 (br, 2H), 1.55 (d, 3H).

### Example 42

### (S)-1-[5-(5-Chloro-2-fluoro-phenyl)-[1,2,4]oxadiazol-3-yl]-ethylamine

The title compound (407 mg, pale yellow oil) was obtained from 5-chloro-2-fluoro-benzoic acid (436 mg, 2.5 mmol). ¹H-NMR (CDCl₃), δ (ppm): 8.12 (dd, 1H), 7.53 (m, 1H), 7.23 (t, 1H), 4.31 (q, 1H), 1.82 (br, s, 2H), 1.57 (d, 3H).

### Example 43

### (S)-1-[5-(3-Chloro-phenyl)-[1,2,4]oxadiazol-3-yl]-ethylamine

(S)-1-[5-(3-Chloro-phenyl)-[1,2,4]oxadiazol-3-yl]-ethylamine(189 mg, light brown oil) was obtained from 3-chlorobenzoic acid (391 mg, 2.5 mmol). ¹H-NMR (CDCl₃), δ (ppm): 8.15 (d, 1H), 8.03 (dd, 1H), 7.57 (t, 1H), 7.48 (dd, 1H), 4.30 (q, 1H), 1.77 (br, s, 2H), 1.57 (d, 3H).

### Example 44

### 1-[5-(3-Chloro-phenyl)-[1,2,4]oxadiazol-3-yl]-propylamine

1-[5-(3-Chloro-phenyl)-[1,2,4]oxadiazol-3-yl]-propylamine (620 mg, yellow oil) was obtained from 3-chlorobenzoic acid (991 mg, 6.33 mmol).). ¹H-NMR (CDCl₃), δ (ppm): 8.15 (d, 1H), 8.03 (dd, 1H), 7.57 (t, 1H), 7.48 (dd, 1H), 4.08 (t, 1H), 1.8-2.2 (m, 4H), 1.0 (t, 3H).

Examples 45-49 were prepared as described for example 13.

### Examples 45a and 45b

### (R)- and (S)-1-[1-(5-(3-Methyl-phenyl)-[1,2,4]oxadiazol-3-yl)-ethyl]-piperazine

(R)-1-[1-(5-(3-Methyl-phenyl)-[1,2,4]oxadiazol-3-yl)-ethyl]-piperazine (71 mg, pale yellow oil) and (S)-1-[1-(5-(3-Methyl-phenyl)-[1,2,4]oxadiazol-3-yl)-ethyl]-piperazine (70 mg, pale yellow oil) were prepred as described in example 13 from the corresponding (R)-1-(5-(3-methyphenylyl-[1,2,4]oxadiazol-3-yl)-ethylamine (203 mg, 1.0 mmol) and (S)-1-(5-(3-methyphenylyl-[1,2,4]oxadiazol-3-yl)-ethylamine (226 mg, 1.1 mmol).

### Example 46

### 1-{1-[5-(3-Chloro-phenyl)-[1,2,4]oxadiazol-3-yl]-propyl}-piperazine

1-{1-[5-(3-Chloro-phenyl)-[1,2,4]oxadiazol-3-yl]-propyl}-piperazine was obtained from 1-[5-(3-Chloro-phenyl)-[1,2,4]oxadiazol-3-yl]-propylamine (190 mg, 0.80 mmol) as in example 13 above. ¹HNMR (CDCL₃) δ (ppm): 8.16 (t, 1H), 8.03 (dd, 1H), 7.56 (dd, 1H), 7.48 (t, 1H), 3.74 (dd, 1H), 2.92 (m, 4H), 2.60 (m, 4H), 2.32 (br, s, 1H), 2.01 (m 2H), 0.93 (t, 3H).

### Example 47

### (S)-1-{1-[5-(3-Chloro-phenyl)-[1,2,4]oxadiazol-3-yl]-ethyl}-piperazine

(S)-1-{1-[5-(3-Chloro-phenyl)-[1,2,4]oxadiazol-3-yl]-ethyl}-piperazine (43 mg, light yellow oil) was obtained from (S)-1-[5-(3-Chloro-phenyl)-[1,2,4]oxadiazol-3-yl]-ethylamine (189 mg, 0.84 mmol) as in example 13 above. ¹H-NMR (CDCl₃), δ (ppm): 8.16 (t, 1H), 8.04 (dd, 1H), 7.56 (dd, 1H), 7.48 (t, 1H), 4.00(q, 1H), 2.93 (m, 4H), 2.61 (m, 4H), 1.66 (br, 1H), 1.55 (d, 3H).

### Example 48

### (S)-1-{1-[5-(5-Chloro-2-fluoro-phenyl)-[1,2,4]oxadiazol-3-yl]-ethyl}-piperazine

(S)-1-{1-[5-(5-Chloro-2-fluoro-phenyl)-[1,2,4]oxadiazol-3-yl]-ethyl}-piperazine was obtained from (S)-1-{1-[5-(5-Chloro-2-fluoro-phenyl)-[1,2,4]oxadiazol-3-yl]-ethylamine (287 mg, 1.19 mmol) as in example 13 above were used as a crude mixture without further purification

### Example 49

### (S)-1-{1-[5-(2-Fluoro-5-methyl-phenyl)-[1,2,4]oxadiazol-3-yl]-ethyl}-piperazine

(S)-1-{1-[5-(2-Fluoro-5-methyl-phenyl)-[1,2,4]oxadiazol-3-yl]-ethyl}-piperazine (91 mg, colorless oil) obtained from (S)-1-{1-[5-(2-Fluoro-5-methyl-phenyl)-[1,2,4]oxadiazol-3-yl]-ethylamine (225 mg, 1.02 mmol) as in example 13 above were used as a crude mixture without further purification.

### Example 50

### 4-(N-Hydroxycarbamimidoylmethyl)-piperazine-1-carboxylic acid ethyl ester

Piperazine-1-carboxylic acid ethyl ester (0.62 mL, 4.2 mmol) was added to a mixture of 2-chloro-N-hydroxy-acetamidine (509 mg, 4.7 mmol) and sodium hydrogen carbonate (820 mg, 9.8 mmol) in acetonitrile (10 mL) and the resulting mixture was stirred at room temperature for 2 days. The reaction mixture was diluted with dichloromethane, filtered through a pad of celite, and concentrated. The title compound (958 mg, %) was obtained by flash column chromatography on silica gel using 90-100% ethyl acetate in hexanes followed by 0-10% methanol in ethyl acetate. ¹H NMR (CDCl₃), δ (ppm): 4.98 (br s, 2H), 4.12 (q, 2H), 3.47 (m, 4H), 2.99 (s, 2H), 2.42 (m, 2H), 1.65 (v br peak, 1H), 1.25 (t, 3H).

### Example 51

### Chloro-hydroxyimino-acetic acid ethyl ester

In 1 L round bottom flask equipped with stir bar added amino-acetic acid ethyl ester hydrochloride (20 g, 143 mmol) and water (30 ml). The solution was cooled down to 0° C. followed by sequential addition of concentrated hydrochloric acid (11.8 ml, 143 mmol) and dropwise addition of sodium nitrite (9.89 g, 143 mmol) solution in water (15 ml). After 10 minutes added another equivalent each of concentrated hydrochloric acid and sodium nitrite solution in water. The reaction mixture was left stirring at 0° C. for 1 h. Reaction mixture was extracted with ether (4×100 ml). Combined organic phase was dried (sodium sulfate), filtered and concentrated in-vacuo to isolate a lemon yellow solid. The solid was recrystallized from hexanes to isolate a white crystalline solid (11 g, 51%). ¹H-NMR (CDCl₃), δ (ppm): 9.98 (bs, 1H), 4.40 (q, 2H), 1.38 (t, 3H).

### Example 52

### 3-Methylsulfanyl-benzoic acid methyl ester

Methyl iodide (0.972 mL) was added to a mixture of 3-mercapto-benzoic acid (601 mg, 3.9 mmol) and potassium carbonate (2.7 g, 19.5 mmol) in DMF (8 mL) in an ice-bath. After the reaction was warmed to room temperature and stirred for 1 hour, the reaction mixture was diluted with ethyl acetate, washed with water (3×), dried over anhydrous sodium sulfate, filtered, and concentrated to afford 3-methylsulfanyl-benzoic acid methyl ester (684 mg, 96%, yellow oil). ¹H NMR (CDCl₃), δ (ppm): 7.90 (s, 1H), 7.80 (d, 1H), 7.44 (d, 1H), 7.35 (t, 1H), 3.92 (s, 3H), 2.53 (s, 3H).

### Example 53

### 3-Methylsulfanyl-benzoic acid

3-Methylsulfanyl-benzoic acid methyl ester (684 mg, 3.8 mmol) and 1N NaOH (5.6 mL, 5.6 mmol) in methanol (8 mL) and THF (8 mL) were heated at 70° C. for 1 hour. The reaction mixture was concentrated and then the residue was diluted with water. After acidification with 1N HCl to pH˜2, the aqueous layer was extracted with ethyl acetate and then washed with water and saturated brine, dried over anhydrous sodium sulfate, filtered, and concentrated to afford 3-methylsulfanyl-benzoic acid (616 mg, 97%, white solid). ¹H NMR (DMSO), δ (ppm): 13.1 (bs, 1H), 7.76 (s, 1H), 7.70 (d, 1H), 7.51 (d, 1H), 7.44 (t, 1H), 2.52 (s, 3H).

### Example 54

### 5-Chloro-2-fluoro-benzoic acid methyl ester

Methanol (20 mL) was added to a solution 5-chloro-2-fluoro-benzoyl chloride (1.2 g, 6.2 mmol) in dichloromethane (10 mL) in an ice-bath. The reaction mixture was warmed to room temperature, stirred for 3 hours and then concentrated to afford 5-chloro-2-fluoro-benzoic acid methyl ester (1.17 g, 100%). ¹H NMR (CDCl₃), δ (ppm): 7.93 (m, 1H), 7.48 (m, 1H), 7.12 (m, 1H), 3.96 (s, 3H).

### Example 55

### 5-Chloro-2-fluoro-benzoic acid hydrazide

A mixture of 5-chloro-2-fluoro-benzoic acid methyl ester (1.17 g, 6.2 mmol) and hydrazine monohydrate (0.451 mL, 9.3 mmol) in ethanol (20 mL) was stirred at room temperature overnight. The reaction mixture was concentrated and then the residue was triturated with diethyl ether to afford 5-chloro-2-fluoro-benzoic acid hydrazide (497 mg, 42%, white solid). ¹H NMR (DMSO), δ (ppm): 9.66 (bs, 1H), 7.58 (m, 2H), 7.36 (m, 1H), 4.58 (bs, 2H).

### Example 56

### 2-Fluoro-5-methyl-benzoic acid hydrazide

HOBt (842 mg, 6.23 mmol), and EDCI (1.19 g, 6.23 mmol) were added to 2-fluoro-5-methyl-benzoic acid methyl ester (800 mg, 5.19 mmol) in acetonitrile (10.3 mL, 197 mmol) at room temperature. After two hours a mixture of hydrazine monohydrate (0.5 mL, 10.38 mmol) in acetonitrile (5.2 mL, 98.6 mmol) and cyclohexene (0.13 mL, 1.28 mmol) was added dropwise at 0° C. After 15 minutes, the solvent was removed using a rotoevaporator and the residue was diluted with ethyl acetate, quenched with water (few mL), washed with sodium carbonate (several times), dried over sodium sulfate, filtered and concentrated to afford 2-fluoro-5-methyl-benzoic acid hydrazide (663 mg, 76%, yellow solid). ¹H NMR (DMSO) δ (ppm): 9.48 (bs, 1H), 7.31 (m, 2H), 7.14 (m, 1H), 4.53 (bs, 2H), 2.30 (s, 3H).

### Example 57

### 2-(5-Chloro-2-fluoro-phenyl)-5-chloromethyl-[1,3,4]oxadiazole

5-Chloro-2-fluoro-benzoic acid hydrazide (188 mg, 1.0 mmol) and 2-chloro-1,1,1-trimethoxy-ethane (1.0 mL) were heated in a sealed vial at 120° C. for 1 hour. The reaction mixture was place directly onto a flash column (silica gel) and purified using 0-7% ethyl acetate in hexanes to afford 2-(5-chloro-2-fluoro-phenyl)-5-chloromethyl-[1,3,4]oxadiazole (180 mg, 73%). ¹H NMR (CDCl₃) δ (ppm): 8.09 (m, 1H), 7.55 (1H), 7.26 (m, 1H), 4.82 (s, 1H).

### Example 58

### 2-(1-Bromo-ethyl)-5-(5-chloro-2-fluoro-phenyl)-[1,3,4]oxadiazole

5-Chloro-2-fluoro-benzoic acid hydrazide (201 mg, 1.1 mmol) and 2-bromo-1,1,1-triethoxypropane (1.09 g, 4.3 mmol) were heated in a sealed vial at 60° C. for 1 hour and then at 120° C. for 30 minutes. The reaction mixture was place directly onto a flash column (silica gel) and purified using 0-50% dichloromethane in hexanes. The product was re-purified by flash column chromatography using a mixture of ethyl acetate:hexanes:dichloromethane (1:19:20) to afford 2-(1-bromo-ethyl)-5-(5-chloro-2-fluoro-phenyl)-[1,3,4]oxadiazole (110 mg, 33%, colorless oil). ¹HNMR (CDCl₃) δ: (ppm): 8.08 (m, 1H), 7.53 (1H), 7.24 (m, 1H), 5.30 (q, 1H), 2.21 (d, 3H).

### Example 59

### 2-Chloromethyl-5-(2-fluoro-5-methyl-phenyl)-[1,3,4]oxadiazole

2-Fluoro-5-methyl-benzoic acid hydrazide (320 mg, 1.9 mmol) and 2-chloro-1,1,1-triethoxy-ethane (1.9 mL) were heated in a sealed vial at 120° C. for 30 minutes. The reaction mixture was place directly onto a flash column (silica gel) and purified by using 0-5% ethyl acetate in hexanes to afford 2-chloromethyl-5-(2-fluoro-5-methyl-phenyl)-[1,3,4]oxadiazole (284.5 mg, 66%). ¹H NMR (CDCl₃) [symbol] (ppm): 7.89 (q, 1H), 7.36 (m, 1H), 7.16 (t, 1H), 4.81 (s, 2H), 2.43 (s, 3H).

### Example 60

### 2-(1-Bromo-ethyl)-5-(2-fluoro-5-methyl-phenyl)-[1,3,4]oxadiazole

2-Fluoro-5-methyl-benzoic acid hydrazide (176 mg, 1.0 mmol) and 2-bromo-1,1,1-triethoxypropane (1.07 g, 4.2 mmol) were heated in a sealed vial at 60° C. for 1 hour and then at 120° C. for 20 minutes. The reaction mixture was place directly onto a flash column (silica gel) and purified using 0-50% dichloromethane in hexanes. The product was re-purified by flash column chromatography using a mixture of ethyl acetate:hexanes:dichloromethane (1:19:20) to afford 2-(1-bromo-ethyl)-5-(2-fluoro-5-methyl-phenyl)-[1,3,4]oxadiazole (81 mg, 27%, colorless oil). ¹H NMR (CDCl₃) [symbol] (ppm): 7.88 (m, 1H), 7.35 (m, 1H), 7.16 (m, 1H), 5.30 (q, 1H), 2.42 (s, 3H), 2.21 (d, 3H).

Examples 61-65 were prepared as described for Example 7.

### Example 61

### 3-Chloromethyl-5-(3-methylsulfanyl-phenyl)-[1,2,4]oxadiazole

3-Chloromethyl-5-(3-methylsulfanyl-phenyl)-[1,2,4]oxadiazole (348 mg, 39% yield over 2 steps, white solid) was obtained from 3-methylsulfanyl-benzoic acid (617 mg, 3.7 mmol), EDCI (773 mg, 4.0 mmol), HOBt (545 mg, 4.0 mmol) and 2-chloro-N-hydroxy-acetamidine (109 mg, 4.0 mmol) in DMF (5 mL). During the initial work-up the acyclic product was also washed with 1N HCl and water and saturated sodium bicarbonate and water and then purified by flash column chromatography eluted with 50-80% ethyl acetate in hexanes. Cyclization in DMF (5 mL) and purification by flash column chromatography using 5% ethyl acetate in hexanes afforded the titled compound. ¹H NMR (CDCl₃), δ (ppm): 8.00 (s, 1H), 7.90 (m, 1H), 7.46 (m, 2H), 4.68 (s, 2H), 2.56 (s, 3H).

### Example 62

### 3-Chloromethyl-5-(2-fluoro-5-methyl-phenyl)-[1,2,4]oxadiazole

3-Chloromethyl-5-(2-fluoro-5-methyl-phenyl)-[1,2,4]oxadiazole (220.4 mg, 36% yield over 2 steps) was obtained from 2-fluoro-5-methyl-benzoic acid (450 mg, 2.92 mmol), EDCI (560 mg, 2.92 mmol), HOBT (447 mg, 2.92 mmol) and 2-chloro-N-hydroxy-acetamidine (293 mg, 2.70 mmol) in DMF (7 mL). The cyclic compound was obtained from heating in DMF (7 mL) and purified by SPE chromatography on silica gel using 300 mL 2% acetone in hexanes. ¹H NMR (CDCl₃), δ (ppm): 7.94 (d, 1H), 7.40 (m, 1H), 7.25 (t, 1H), 4.71 (s, 2H), 2.42 (s, 3H).

### Example 63

### 3-Chloromethyl-5-(2-fluoro-5-bromo-phenyl)-1,2,4]oxadiazole

3-Chloromethyl-5-(2-fluoro-5-bromo-phenyl)-[1,2,4]oxadiazole (280.1 mg, 50.6% yield over 2 steps) was obtained from 2-fluoro-5-bromo-benzoic acid (450 mg, 2.055 mmol), EDCI (393.9 mg, 2.055 mmol), HOBT (314.7 mg, 2.055 mmol) and 2-chloro-N-hydroxy-acetamidine (206.2 mg, 1.9 mmol) in DMF (7 mL). The cyclic compound was obtained from heating in DMF (7 mL) and purified by SPE chromatography on silica gel using 250 mL 10% ethyl acetate in hexanes. ¹H NMR (CDCl₃), δ (ppm): 8.32 (m, 1H), 7.73 (m, 1H), 7.22 (q, 1H), 4.72 (s, 2H).

### Example 64

### 3-Chloromethyl-5-(2,5-dichloro-phenyl)-[1,2,4]oxadiazole

3-Chloromethyl-5-(2,5-dichloro-phenyl)-[1,2,4]oxadiazole (287.4 mg, 63.9% yield over 2 steps) was obtained from 2,5-dichloro-benzoic acid (450 mg, 2.36 mmol), EDCI (452 mg, 2.36 mmol), HOBT (361.4 mg, 2.36 mmol) and 2-chloro-N-hydroxy-acetamidine (230 mg, 2.12 mmol) in DMF (5 mL). The cyclic compound was obtained from heating in DMF (5 mL) and purified by SPE chromatography on silica gel using 250 mL 10% acetone in hexanes. ¹H NMR (CDCl₃), δ (ppm): 8.13 (m, 1H), 7.52 (m, 2H), 4.72 (s, 2H).

### Example 65

### 5-(5-Chloro-2-fluoro-phenyl)-3-chloromethyl-[1,2,4]oxadiazole

5-(5-chloro-2-fluoro-phenyl)-3-chloromethyl-[1,2,4]oxadiazole (438 mg, 56%, white solid) was prepared from 2-fluoro-5-chlorobenzoic acid (550 mg, 3.15 mmol), EDCI (665 mg, 3.47 mmol), HOBT (469 mg, 3.47 mmol) and 2-chloro-N-hydroxy-acetamidine (377 mg, 3.47 mmol) in DMF (10 mL). To effect cyclization to oxadiazole, DMF (15 mL) was added to the intermediate residue and the mixture was heated for 1 hour. Purification of the title compound was performed by flash column chromatography using 10% ethyl acetate in hexanes. ¹H NMR (CDCl₃) δ (ppm): 8.16 (m, 1H), 7.58 (m, 1H), 7.29 (m, 1H), 4.72 (s, 3H).

### Example 66

### 3-Chloromethyl-5-(2-chloro-5-methyl-phenyl)-[1,2,4]oxadiazole

2-Chloro-5-methyl-benzoic acid (1 g, 5.8 mmol) was treated with thionyl chloride (5 mL) at reflux for two hours. Excess thionyl chloride was removed under reduced pressure. The residue was added to a suspension of 2-chloro-N-hydroxy-acetamidine (638 mg, 5.8 mmol) in dichloromethane (10 mL) at room temperature. After stirring for 30 minutes, triethylamine (2.04 mL, 14.6 mmol) was added and stirred for an additional hour. The reaction mixture was diluted with ethyl acetate, washed with water and brine, dried over anhydrous sodium sulfate, filtered and concentrated. Flash column chromatography using 10-20% ethyl acetate in hexanes afforded 460 mg of the acyclic ester intermediate. DMF was added to this intermediate and then heated at 135° C. for 4 h to effect cyclization to oxadiazole. After cooling the reaction mixture was washed with water (3 times) and brine, dried over anhydrous sodium sulfate, filtered, and concentrated. Purification by flash column chromatography on silica gel using 5% ethyl acetate in hexanes afforded the title compound 160 mg (12% over 2 steps) as a white solid. m/z 244 (GCMS)

### Example 67

### 5-(3-Chloro-phenyl)-[1,2,4]oxadiazole-3-carboxylic acid ethyl ester

To a mixture of(3-chloro-benzoylamino)-acetic acid (9.0 g, 42.1 mmol) with POCl₃ (16.1 g, 105.3 mmol) under ice bath, DMF (7.08 g, 96.8 mmol) was added with vigorous stirring. After being heated at 50° C. for an hour, the reaction mixture was poured into ice. The precipitate was filtered and washed with water to give 10.5 g (quantitative) of 2-(3-chloro-phenyl)-4-dimethylaminomethylene-4H-oxazol-5-one as pale-orange solid. 1H-NMR(CDCl₃) δ (ppm): 7.96 (s, 1H), 7.82 (d, 1H), 7.39 (m, 2H), 7.16 (s, 1H), 3.64 (s, 3H) and 3.28 (s, 3H).

2-(3-Chloro-phenyl)-4-dimethylaminomethylene-4H-oxazol-5-one (10.5, 41.9 mmole) was heated with sodium hydroxide (0.8 g, 20 mmol) in ethanol (120 mL) at reflux for 30 minutes. The reaction mixture was concentrated and the residue was mixed with 4% HCl (100 mL) and ether (100 mL). NaNO2 (3.6 g, 52.2 mmol) in water (20 mL) was added dropwise. The reaction mixture was stirred vigorously overnight. The mixture was filtered through celite and washed with ether. The ether layer was washed with water and brine, concentrated, purified by column chromatography with dichloromethane to give 6.5 g (61.4%) of 5-(3-Chloro-phenyl)-[1,2,4]oxadiazole-3-carboxylic acid ethyl ester as pale-yellow oil. ¹H-NMR(CDCl₃) δ (ppm): 8.26 (s, 1H), 8.13 (d, 1H), 7.64 (d, 1H), 7.53 (t, 1H), 4.58 (q, 2H) and 1.50 (t, 3H).

### Example 68

### 5-(3-Chloro-phenyl)-[1,2,4]oxadiazole-3-carbaldehyde

5-(3-chloro-phenyl)-[1,2,4]oxadiazole-3-carboxylic acid ethyl ester (4 g, 15.83 mmol) in dichloromethane (30 mL) was cooled to −78° C. DIBAL-H (1M Hexanes, 28.5 mL, 28.5 mmol) was added dropwise and the reaction was left stirring at −78° C. for 40 minutes. After the reaction was quenched with water (30 mL) and Rochelle salt solution (50 mL) at 0° C., the reaction was warmed to room temperature and left stirring overnight. The reaction mixture was filtered through celite and then the organic layer was separated, dried over sodium sulfate, filtered, and concentrated. The residue was purified by flash column chromatography on silica gel using 0-15% ethyl acetate in dichlormethane to afford 5-(3-chloro-phenyl)-[1,2,4]oxadiazole-3-carbaldehyde (0.84 g, 25%, white solid). ¹H NMR (CDCl₃) [symbol] (ppm): 10.23 (s, 1H), 8.26 (m, 1H), 8.15 (m, 1H), 7.65 (m, 1H), 7.55 (m, 1H).

### Example 69

### 1-[5-(3-Chloro-phenyl)-[1,2,4]oxadiazol-3-yl]-ethanol

Under argon, CH₃MgI (4.0 mL, 12.08 mmol) was added drop-wise to a solution of 5-(3-chloro-phenyl)-[1,2,4]oxadiazole-3-carbaldehyde (0.84 g, 4.03 mmol) in THF (10 mL) at 0° C. The reaction mixture was left stirring at 0° C. for 1.75 hours. After 1N hydrochloric acid (20 mL) was added slowly to the reaction mixture, the reaction mixture was extracted with diethyl ether (3×50 mL). The organic layer was dried over anhydrous sodium sulfate, filtered, and concentrated. Purification with flash column chromatography on silica gel using 0-30% ethyl acetate in hexanes afforded 1-[5-(3-chloro-phenyl)-[1,2,4]oxadiazol-3-yl]-ethanol (0.4478 g, 50%). ¹H NMR (CDCl₃) [symbol] (ppm): 8.16 (m, 1H), 8.05 (m, 1H), 7.58 (m, 1H), 7.53 (m, 1H), 5.10 (q, 1H), 2.53 (d, 1H), 1.69 (d, 3H).

### Example 70

### Methanesulfonic acid 1-[5-(3-chloro-phenyl)-[1,2,4]oxadiazol-3-yl]-ethyl ester

To 1-[5-(3-chloro-phenyl)-[1,2,4]oxadiazol-3-yl]-ethanol (448 mg, 1.99 mmol) in dichloromethane (10 mL) at 0° C., triethyl amine (1.39 mL, 9.97 mmol) and methanesulfonyl chloride (0.46 mL, 5.98 mmol) were added. After one hour, the reaction mixture was quenched with water (30 mL) and left to stir at 0° C. for another hour. The organic phase was separated, washed with 1N hydrochloric acid, sodium bicarbonate and brine. The organic layer was then dried over anhydrous sodium sulfate, filtered and concentrated in vacuo to afford methanesulfonic acid 1-[5-(3-chloro-phenyl)-[1,2,4]oxadiazol-3-yl]-ethyl ester (656 mg, light brown solid). ¹H NMR (CDCl₃) [symbol] (ppm): 8.16 (m, 1H), 8.05 (m, 1H), 7.62 (m, 1H), 7.52 (m, 1H), 5.95 (q, 1H), 3.16 (s, 3H), 1.90 (d, 3H).

### Example 71

### 4-(3-Chloro-phenyl)-2,4-dioxo-butyric acid ethyl ester

Sodium hydride (60% oil dispersion, 1.24 g, 31.1 mmol) was added in portions to a solution of 3-chloroacetophenone (4.0 g, 25.9 mmol) and diethyl oxalate (4.54 g, 31.1 mmol) in DMF (32 mL) at 0° C. The mixture stirred at room temperature for 1 hour and was then heated at 80° C. for a half an hour. After cooling, the mixture was treated with 3N HCl and then diluted with ethyl acetate. The organic layer was washed with water (3×) and saturated brine, dried over anhydrous sodium sulfate, filtered and concentrated. The resulting residue was then purified by flash column chromatography on silica using 0-10% ethyl acetate in hexanes to afford of 4-(3-chloro-phenyl)-2,4-dioxo-butyric acid ethyl ester (4.43 g, 67%, yellow solid). I H NMR (CDCl₃) δ (ppm): 15.12 (br s, 1H), 7.98 (s, 1H), 7.88 (d, 1H), 7.58 (d, 1H), 7.47 (t, 1H), 7.05 (s, 1H), 4.39 (m, 2H), 1.41 (m, 3H).

### Example 72

### 5-(3-Chloro-phenyl)-isoxazole-3-carboxylic acid ethyl ester

A solution of 4-(3-chloro-phenyl)-2,4-dioxo-butyric acid ethyl ester (3.0 g, 11.8 mmol) and hydroxylamine hydrochloride (2.46 g, 35.4 mmol) in methanol (60 mL) was heated at 80° C. for 4 hours. After cooling, the mixture was filtered and washed with cold methanol to afford 5-(3-chloro-phenyl)-isoxazole-3-carboxylic acid ethyl ester (2.0 g, 71%, white solid). ¹H NMR (CDCl₃) δ (ppm): 7.82 (s, 1H), 7.72 (m, 1H), 7.47 (m, 2H), 4.03 (s, 3H). Mixture of both methyl and ethyl ester (mostly methyl).

### Example 73

### [5-(3-Chloro-phenyl)-isoxazol-3-yl]-methanol

Lithium aluminum hydride (320 mg, 8.4 mmol) was slowly added to a solution of 5-(3-chloro-phenyl)-isoxazole-3-carcoxylic acid ethyl ester (2.0 g, 8.4) in THF (100 mL) at room temperature. After 1 hour, the reaction mixture was quenched with water and then extracted with ethyl acetate. The organic layer was washed with water and saturated brine, dried over anhydrous sodium sulfate, filtered, and concentrated. The resulting residue was then purified by flash column chromatography using 15-40% ethyl acetate in hexane to afford [5-(3-chloro-phenyl)-isoxazol-3-yl]-methanol (1.32 g, 75%, yellow solid). ¹H NMR (CDCl₃) δ (ppm): 7.78 (s, 1H), 7.68 (m, 1H), 7.43 (m, 2H), 6.63 (s, 1H), 4.84 (d, 2H), 2.23 (t, 1H).

### Example 74

### Methanesulfonic acid 5-(3-chloro-phenyl)-isoxazol-3-ylmethyl ester

Triethyl amine (965 mg, 9.5 mmol) and methanesulfonyl chloride (820 mg, 7.2 mmol) were added to a solution of [5-(3-chloro-phenyl)-isoxazol-3-yl]-methanol (1.0 g, 4.8 mmol) in dichloromethane (50 mL) at 0° C. After 1 hour, the reaction mixture was quenched with cold saturated sodium bicarbonate and then the organic layer was washed with saturated brine, dried over anhydrous sodium sulfate, filtered, and concentrated to afford methanesulfonic acid 5-(3-chloro-phenyl)-isoxazol-3-ylmethyl ester (1.4 g, 100%, light brown solid). ¹H NMR (CDCl₃) δ (ppm): 7.80 (s, 1H), 7.70 (m, 1H), 7.45 (m, 2H), 6.73 (s, 1H), 5.37 (s, 2H), 3.16 (s, 3H).

### Example 75

### 1-[5-(3-Chloro-phenyl)-isoxazol-3-yl]-ethanone

In a screw cap vial equipped with stir bar added methyl magnesium iodide (3M in diethyl ether) (0.79 ml, 2.38 mmol), toluene (1 ml), tetrahydrofuran (0.39 ml, 4.77 mmol) and triethylamine (1 ml, 7.15 mmol). Cooled the solution down to 0° C. and to it added solution of 5-(3-chloro-phenyl)-isoxazole-3-carboxylic acid ethyl ester (300 mg, 1.19 mmol) in toluene (5 ml). Left the resulting mixture stirring at 0° C. for 5 h. Reaction mixture was quenched with 1N hydrochloric acid (aqueous, 6.5 ml, 6.5 mmol), diluted with toluene (35 ml), sequentially washed with water (50 ml), saturated sodium bicarbonate (aqueous, 30 ml), water (50 ml) and brine (30 ml). The organic phase was concentrated, in-vacuo. The isolated residue was dissolved in methanol (8 ml) and 20% potassium hydroxide (aqueous, 1 ml). The mixture was stirred at 45° C. for 30 minutes. At this point the mixture was concentrated, in-vacuo. The isolated residue was dissolved in toluene (60 ml), sequentially washed with water (50 ml), saturated sodium bicarbonate (aqueous, 50 ml) and water (50 ml). The organic phase was concentrated, in-vacuo. The crude residue was purified on silica gel using 2% ethyl acetate in hexanes to isolate the desired compound as a white solid (156 mg, 60%). ¹H-NMR (CDCl₃), δ (ppm): 7.77 (m, 1H), 7.66 (m, 1H), 7.42 (m, 2H), 6.90 (s, 1H), 2.69 (s, 3H).

### Example 76

### Methanesulfonic acid 1-[5-(3-Chloro-phenyl)-isoxazol-3-yl]-ethyl ester

In a screw cap vial equipped with stir bar added 1-[5-(3-chloro-phenyl)-isoxazol-3-yl]-ethanone (100 mg, 0.45 mmol), sodium borohydride (34 mg, 0.90 mmol) and methanol (3 ml). Left the resulting mixture stirring at room temperature for 3 h. Reaction was quenched with water (30 ml) and brine (30 ml), extracted with dichloromethane (3×30 ml). Combined organic phase was dried (sodium sulfate), filtered and concentrated, in-vacuo to isolate 1-[5-(3-Chloro-phenyl)-isoxazol-3-yl]-ethanol as a white solid (110 mg). ¹H-NMR (CDCl₃), δ (ppm): 7.69 (m, 1H), 7.59 (m, 1H), 7.37 (m, 2H), 6.59 (s, 1H), 5.07 (q, 1H), 3.45 (bs, 1H), 1.58 (d, 3H). In a screw cap vial equipped with stir bar added the isolated alcohol (110 mg, 0.49 mmol), dichloromethane (3 ml) and triethylamine (0.34 ml, 2.46 mmol). Cooled the mixture down to 0° C. and to it added methane sulfonyl chloride (0.08 ml, 0.98 mmol). Left the reaction mixture stirring at room temperature for 30 minutes. Reaction was quenched with saturated sodium bicarbonate (aqueous, 40 ml) and extracted with dichloromethane (3×30 ml). Combined organic phase was washed with brine (40 ml), dried (sodium sulfate), filtered and concentrated, in-vacuo to isolate the desired compound as brown oil.

### Example 77

### 4-(2-Fluoro-5-methyl-phenyl)-2,4-dioxo-butyric acid methyl ester

Sodium hydride (60% oil dispersion, 948 mg, 23.7 mmol) was added in portions to a solution of 2′-fluoro-5′-methylacetophenone (3.0 g, 19.7 mmol) and dimethyl oxalate (2.80 g, 23.7 mmol) in DMF (32 mL) at 0° C. The mixture stirred at 80° C. for a half an hour. After cooling, the mixture was treated with 3N HCl and then diluted with ethyl acetate. The organic layer was washed with water (3×) and saturated brine, dried over anhydrous sodium sulfate, filtered and concentrated. Trituration of the residue with 1% ethyl acetate/hexanes and then filtration afforded 4-(2-fluoro-5-methyl-phenyl)-2,4-dioxo-butyric acid methyl ester (2.1 g, 45%, brown solid). ¹H NMR (CDCl₃) δ (ppm): 15.15 (bs, 1H), 7.76 (m, 1H), 7.37 (m, 1H), 7.14 (s, 1H), 7.08 (t, 1H), 3.94 (s, 3H), 2.40 (s, 3H).

### Example 78

### 5-(2-Fluoro-5-methyl-phenyl)-isoxazole-3-carboxylic acid methyl ester

A solution 4-(2-fluoro-5-methyl-phenyl)-2,4-dioxo-butyric acid methyl ester (2.1 g, 8.8 mmol) and hydroxylamine hydrochloride (1.8 g, 26.4 mmol) in methanol (45 mL) was heated at 80° C. for 30 minutes. After cooling, the mixture was concentrated and then diluted with ethyl acetate, washed with water and saturated brine, dried over anhydrous sodium sulfate, filtered, and concentrated. Purification by flash column chromatography on silica gel using 10% ethyl acetate in hexanes afforded 5-(2-fluoro-5-methyl-phenyl)-isoxazole-3-carboxylic acid methyl ester (1.7 g, 80%, light brown solid). ¹H NMR (CDCl₃) δ (ppm): 7.81 (m, 1H), 7.26 (m, 1H), 7.12 (m, 2H), 4.03 (s, 3H), 2.43 (s, 3H).

### Example 79

### [5-(2-Fluoro-5-methyl-phenyl)-isoxazol-3-yl]-methanol

Lithium aluminum hydride (129 mg, 3.4 mmol) was slowly added to a solution of 5-(2-fluoro-5-methyl-phenyl)-isoxazole-3-carboxylic acid methyl ester (800 mg, 3.4) in THF (35 mL) at room temperature. After 1 hour, the reaction mixture was quenched with water and then extracted with ethyl acetate. The organic layer was washed with water and saturated brine, dried over anhydrous sodium sulfate, filtered, and concentrated to afford the [5-(2-fluoro-5-methyl-phenyl)-isoxazol-3-yl]-methanol (694 mg, 98%, light yellow solid). ¹H NMR (CDCl₃) δ (ppm): 7.76 (m, 1H), 7.22 (m, 1H), 7.09 (m, 1H), 6.77 (d, 1H), 4.86 (d, 2H), 2.41 (s, 3H), 2.05 (t, 1H).

### Example 80

### Methanesulfonic acid 5-(2-fluoro-5-methyl-phenyl)-isoxazol-3-ylmethyl ester

Triethyl amine (0.933 mL, 6.7 mmol) and methanesulfonyl chloride (0.389 mL, 5.0 mmol) were added to a solution of [5-(2-fluoro-5-methyl-phenyl)-isoxazol-3-yl]-methanol (694 mg, 3.4 mmol) in dichloromethane (35 mL) at 0° C. After 1 hour, the reaction mixture was quenched with cold saturated sodium bicarbonate and then the organic layer was washed with saturated brine, dried over anhydrous sodium sulfate, filtered, and concentrated to afford methanesulfonic acid 5-(2-fluoro-5-methyl-phenyl)-isoxazol-3-ylmethyl ester (943 mg, 99%, light brown solid). ¹H NMR (CDCl₃) δ (ppm): 7.77 (m, 1H), 7.25 (m, 1H), 7.11 (m, 1H), 6.85 (d, 1H), 5.38 (s, 2H), 3.12 (s, 3H), 2.42 (s, 3H).

### Example 81

### 1-[5-(2-Fluoro-5-methyl-phenyl)-isoxazol-3-yl]-ethanone

In a screw cap vial equipped with stir bar added methyl magnesium iodide (3M in diethyl ether) (1.1 ml, 3.40 mmol), toluene (1 ml), tetrahydrofuran (0.55 ml, 6.80 mmol) and triethylamine (1.42 ml, 10.2 mmol). Cooled the solution down to 0° C. and to it added solution of 5-(2-fluoro-5-methyl-phenyl)-isoxazole-3-carboxylic acid methyl ester (400 mg, 1.70 mmol) in toluene (6 ml). Left the resulting mixture stirring at 0° C. for 3 h. Reaction mixture was quenched with 1N hydrochloric acid (aqueous, 50 ml) and extracted with diethyl ether (2×50 ml). Combined the organic phase was washed with brine (50 ml), dried (sodium sulfate), filtered and concentrated, in-vacuo. The crude residue was purified on silica gel using 2% diethyl ether in hexanes to isolate the desired compound as a yellow solid (220 mg, 59%). ¹H-NMR (CDCl₃), δ (ppm): 7.79 (dd, 1H), 7.25 (m, 1H), 7.08 (m, 2H), 2.73 (s, 3H), 2.43 (s, 3H).

### Example 82

### Methanesulfonic acid 1-[5-(2-fluoro-5-methyl-phenyl)-isoxazol-3-yl]-ethyl ester

In a screw cap vial equipped with stir bar added 1-[5-(2-fluoro-5-methyl-phenyl)-isoxazol-3-yl]-ethanone (220 mg, 1.00 mmol), sodium borohydride (76 mg, 2.01 mmol) and methanol (5 ml). Left the resulting mixture stirring at room temperature for 3 h. Reaction was quenched with water (30 ml) and brine (30 ml), extracted with dichloromethane (3×30 ml). Combined organic phase was dried (sodium sulfate), filtered and concentrated, in-vacuo to isolate 1-[5-(2-fluoro-5-methyl-phenyl)-isoxazol-3-yl]-ethanol as yellow oil. ¹H-NMR (CDCl₃), δ (ppm): 7.77 (dd, 1H), 7.23 (m, 1H), 7.09 (m, 1H), 6.74 (d, 1H), 5.13 (m, 1H), 2.41 (s, 3H), 2.20 (d, 1H), 1.63 (d, 3H). The isolated alcohol was dissolved in dichloromethane (3 ml) and triethylamine (0.70 ml, 5.01 mmol) was added. Cooled the mixture down to 0° C. and to it added methane sulfonyl chloride (0.16 ml, 2.01 mmol). Left the reaction mixture stirring at room temperature for 30 minutes. Reaction was quenched with saturated sodium bicarbonate (aqueous, 40 ml) and extracted with dichloromethane (3×30 ml). Combined organic phase was washed with brine (40 ml), dried (sodium sulfate), filtered and concentrated, in-vacuo to isolate the desired compound as brown oil (327 mg).

### Example 83

### 1-[5-(2-Fluoro-5-methyl-phenyl)-isoxazol-3-yl]-propan-1-one

In a screw cap vial equipped with stir bar added ethyl magnesium bromide (3M in diethyl ether) (0.85 ml, 2.55 mmol), toluene (1 ml), tetrahydrofuran (0.41 ml, 5.10 mmol) and triethylamine (1.07 ml, 7.65 mmol). Cooled the solution down to 0° C. and to it added solution of 5-(2-fluoro-5-methyl-phenyl)-isoxazole-3-carboxylic acid methyl ester (300 mg, 1.28 mmol) in toluene (5 ml). Left the resulting mixture stirring at 0° C. for 3 h. Reaction mixture was quenched with 1N hydrochloric acid (aqueous, 50 ml) and extracted with diethyl ether (2×50 ml). Combined the organic phase was washed with brine (50 ml), dried (sodium sulfate), filtered and concentrated, in-vacuo. The crude residue was purified on silica gel using 2% diethyl ether in hexanes to isolate the desired compound as yellow oil (40 mg). ¹H-NMR (CDCl₃), δ (ppm): 7.77 (dd, 1H), 7.25 (m, 1H), 7.09 (m, 2H), 3.15 (q, 2H), 2.41 (s, 3H), 1.25 (t, 3H).

### Example 84

### Methanesulfonic acid 1-[5-(2-fluoro-5-methyl-phenyl)-isoxazol-3-yl]-propyl ester

In a screw cap vial equipped with stir bar added 1-[5-(2-fluoro-5-methyl-phenyl)-isoxazol-3-yl]-propan-1-one (37 mg, 0.16 mmol), sodium borohydride (12 mg, 0.32 mmol) and methanol (2 ml). Left the resulting mixture stirring at room temperature for 3 h. Reaction was quenched with water (15 ml) and brine (15 ml), extracted with dichloromethane (3×15 ml). Combined organic phase was dried (sodium sulfate), filtered and concentrated, in-vacuo to isolate 1-[5-(2-fluoro-5-methyl-phenyl)-isoxazol-3-yl]-propan-1-ol as yellow oil. The isolated alcohol (38 mg, 0116 mmol) was dissolved in dichloromethane (2 ml) and triethylamine (0.11 ml, 0.79 mmol) was added. Cooled the mixture down to 0° C. and to it added methane sulfonyl chloride (0.02 ml, 0.32 mmol). Left the reaction mixture stirring at room temperature for 30 minutes. Reaction was quenched with saturated sodium bicarbonate (aqueous, 20 ml) and extracted with dichloromethane (3×15 ml). Combined organic phase was washed with brine (20 ml), dried (sodium sulfate), filtered and concentrated, in-vacuo to isolate methanesulfonic acid 1-[5-(2-fluoro-5-methyl-phenyl)-isoxazol-3-yl]-propyl ester as brown oil.

### Example 85

### Methanesulfonic acid cyclopropyl-[5-(2-fluoro-5-methyl-phenyl)-isoxazol-3-yl]-methyl ester

In a screw cap vial equipped with stir bar added 5-(2-fluoro-5-methyl-phenyl)-isoxazole-3-carbaldehyde (0.13 g, 0.61 mmol) and tetrahydrofuran (2 ml). Cooled the mixture down to 0° C. and to it added methyl cyclopropyl magnesium bromide (0.5M in tetrahydrofuran, 3.7 ml, 1.83 mmol). The resulting mixture was left stirring at 0° C. for 4 h. Reaction mixture was quenched with hydrochloric acid (1N, aqueous, 10 ml), extracted with diethyl ether (3×50 ml). Combined organic phase was washed with water (50 ml), brine (50 ml), dried (sodium sulfate), filtered and concentrated in-vacuo. The crude residue was purified on silica gel using 10% ethyl acetate in hexanes to isolate cyclopropyl-[5-(2-fluoro-5-methyl-phenyl)-isoxazol-3-yl]-methanol as clear oil (121 mg, 80%). ¹H-NMR (CDCl₃), δ (ppm): 7.67 (dd, 1H), 7.14 (m, 1H), 7.01 (dt, 1H), 6.76 (d, 1H), 4.26 (dd, 1H), 3.45 (d, 1H), 2.34 (s, 3H), 1.29 (m, 1H), 0.58 (m, 4H). In a screw cap vial equipped with stir bar added the isolated alcohol (121 mg, 0.49 mmol), dichloromethane (3 ml) and triethylamine (0.34 ml, 2.45 mmol). Cooled the mixture down to 0° C. and to it added methane sulfonyl chloride (0.1 ml, 0.98 mmol). Left the reaction mixture stirring at room temperature for 30 minutes. Reaction was quenched with saturated sodium bicarbonate (aqueous, 40 ml) and extracted with dichloromethane (3×30 ml). Combined organic phase was washed with brine (40 ml), dried (sodium sulfate), filtered and concentrated, in-vacuo to isolate the title compound as brown oil (160 mg).

### Example 86

### (5-Chloro-2-fluoro-phenylethynyl)-trimethyl-silane

In a 250 mL round bottom flask equipped with a stir bar and reflux condenser added 4-bromo-2-chloro-1-fluoro-benzene (5 g, 23.9 mmol), triphenylphosphine (250 mg, 0.10 mmol), (trimethylsilyl)acetylene (5.2 ml, 36.5 mmol)and triethylamine (60 ml). The reaction mixture was purged with argon, followed by addition of palladium (II) acetate (108 mg, 0.05 mmol). The resulting mixture was left stirring at reflux under argon, overnight. The reaction mixture was filtered through a pad of celite using ethyl acetate and the filtrate was concentrated in-vacuo. The isolated residue was absorbed on silica gel and filtered using hexanes. The filtrate was concentrated in-vacuo to isolate the title compound as brown oil (5.42 g).

### Example 87

### 4-Chloro-2-ethynyl-1-fluoro-benzene

In a 250 mL round bottom flask equipped with stir bar added (5-chloro-2-fluoro-phenylethynyl)-trimethyl-silane (5.42 g, 23.9 mmol), potassium carbonate (16.5 g, 120 mmol) and methanol (60 ml). The reaction mixture was left stirring at room temperature for 1 h. Diluted the reaction mixture with hexanes (200 ml) and washed with water (250 ml). The aqueous phase was extracted with hexanes (2×100 ml). Combined organic phase was washed with brine (200 ml), dried (sodium sulfate), filtered and concentrated in-vacuo to isolate the desired compound as brown oil (3.56 g). ¹H-NMR (CDCl₃), δ (ppm): 7.47 (dd, 1H), 7.30 (m, 1H), 7.05 (t, 1H), 3.36 (s, 1H).

### Example 88

### 5-(5-Chloro-2-fluoro-phenyl)-isoxazole-3-carboxylic acid ethyl ester

In a 250 mL round bottom flask equipped with stir bar added 4-bromo-2-ethynyl-1-fluoro-benzene (2 g, 12.9 mmol), chloro-hydroxyimino-acetic acid ethyl ester (3.92 g, 25.9 mmol), sodium bicarbonate (7.07 g, 84.1 mmol) and toluene (50 ml). Reaction mixture was left stirring at room temperature for 48 h, after which it was concentrated in-vacuo. Residue was taken up in ethyl acetate (200 ml), sequentially washed with water (150 ml), brine (150 ml), dried (sodium sulfate), filtered and concentrated in-vacuo. The crude residue was purified on silica gel using 3% acetone in hexanes to isolate the title compound as an off-white solid (1.56 g). ¹H-NMR (CDCl₃), δ (ppm): 8.00 (dd, 1H), 7.43 (m, 1H), 7.18 (m, 2H), 4.51 (q, 2H), 1.47 (t, 3H).

### Example 89

### [5-(5-Chloro-2-fluoro-phenyl)-isoxazol-3-yl]-methanol

In a 50 mL round bottom flask equipped with stir bar and drying tube added 5-(5-chloro-2-fluoro-phenyl)-isoxazole-3-carboxylic acid ethyl ester (0.78 g, 2.89 mmol) and tetrahydrofuran (10 ml). To this stirred solution added solution of lithium aluminum hydride (0.12 g, 2.89 mmol) in tetrahydrfuran (2 ml). The resulting mixture was left stirring at room temperature for 1 h. Reaction was quenched using sodium sulfate decahydrate. The resulting mixture was stirred at 63° C. for 15 minutes after which it was filtered through a celite pad. The filtrate was concentrated in-vacuo to isolate the title compound as yellow solid (0.65 g, 99%). ¹H-NMR (CDCl₃), δ (ppm): 7.73 (dd, 1H), 7.27 (m, 1H), 7.24 (t, 1H), 6.73 (d, 1H), 4.77 (s, 2H), 4.45 (bs, 1H).

### Example 90

### 5-(5-Chloro-2-fluoro-phenyl)-isoxazole-3-carbaldehyde

In a 50 mL round bottom flask equipped with stir bar and drying tube added 5-(5-chloro-2-fluoro-phenyl)-isoxazole-3-carboxylic acid ethyl ester (0.78 g, 2.89 mmol) and dichloromethane (10 ml). Cooled the solution down to −78° C. and to this stirred solution added diisobutylaluminum hydride (1M hexanes, 5.3 ml, 5.3 mmol). The resulting mixture was left stirring at −78° C. for 3 h. Reaction was quenched using sodium sulfate decahydrate. The resulting mixture was stirred at 63C for 15 minutes after which it was filtered through a celite pad. The filtrate was concentrated in-vacuo to isolate an off-white solid, which was triturated with hexanes to isolate the title compound as a white solid (0.55 g, 84%). ¹H-NMR (CDCl₃), δ (ppm): 10.2 (s, 1H), 7.99 (m, 1H), 7.44 (m, 1H), 7.20 (m, 1H), 7.10 (d, 1H).

### Example 91

### 1-[5-(5-Chloro-2-fluoro-phenyl)-isoxazol-3-yl]-ethanol

In a 50 mL round bottom flask equipped with stir bar added 5-(5-chloro-2-fluoro-phenyl)-isoxazole-3-carbaldehyde (0.55 g, 2.42 mmol) and tetrahydrofuran (6 ml). Cooled the mixture down to 0° C. and to it added methyl magnesium iodide (3M in diethyl ether, 3.23 ml, 9.67 mmol). The resulting mixture was left stirring at 0° C. for 3 h. Reaction mixture was quenched with hydrochloric acid (1N, aqueous, 10 ml), extracted with diethyl ether (3×50 ml). Combined organic phase was washed with water (50 ml), brine (50 ml), dried (sodium sulfate), filtered and concentrated in-vacuo. The crude residue was purified on silica gel using 10% ethyl acetate in hexanes to isolate the desired compound as clear oil (179 mg, 31%).

### Example 92

### Methanesulfonic acid 5-(5-chloro-2-fluoro-phenyl)-isoxazol-3-ylmethyl ester

In a screw cap vial equipped with stir bar added [5-(5-chloro-2-fluoro-phenyl)-isoxazol-3-yl]-methanol (296 mg, 1.3 mmol), dichloromethane (5 ml) and triethylamine (1.81 ml, 13 mmol). Cooled the mixture down to 0° C. and to it added methane sulfonyl chloride (0.4 ml, 5.19 mmol). Left the reaction mixture stirring at room temperature for 30 minutes. Reaction was quenched with saturated sodium bicarbonate (aqueous, 40 ml) and extracted with dichloromethane (3×30 ml). Combined organic phase was washed with brine (40 ml), dried (sodium sulfate), filtered and concentrated, in-vacuo to isolate the desired compound as brown oil (345 mg).

### Example 93

### Methanesulfonic acid 1-[5-(5-chloro-2-fluoro-phenyl)-isoxazol-3-yl]-ethyl ester

In a screw cap vial equipped with stir bar added 1-[5-(5-chloro-2-fluoro-phenyl)-isoxazol-3-yl]-ethanol (190 mg, 0.79 mmol) and dichloromethane (5 ml) and triethylamine (1.1 ml, 7.86 mmol). Cooled the mixture down to 0° C. and to it added methane sulfonyl chloride (0.24 ml, 3.15 mmol). Left the reaction mixture stirring at room temperature for 30 minutes. Reaction was quenched with saturated sodium bicarbonate (aqueous, 40 ml) and extracted with dichloromethane (3×30 ml). Combined organic phase was washed with brine (40 ml), dried (sodium sulfate), filtered and concentrated, in-vacuo to isolate the desired compound as brown oil (301 mg).

### Example 94

### 2,4-Dioxo-4-thiophen-3-yl-butyric acid methyl ester

Sodium hydride (60% oil dispersion, 1.9 g, 47.6 mmol) was added to a solution of 3-acetylthiophene (5.0 g, 39.6 mmol) and dimethyl oxalate (5.6 g, 47.6 mmol) in DMF (32 mL) at 0° C. The mixture stirred at room temperature for 1 hour and was then quenched with 3N HCl. After diluting with ethyl acetate, the organic layer was washed with water (3×) and saturated brine, dried over anhydrous sodium sulfate, filtered and concentrated. The resulting residue was then purified by triturating with 1% ethyl acetate in hexanes to afford the titled compound (7.54 g, 90%, light pink solid). 1H NMR (CDCl₃) δ (ppm): 15.90 (br s, 1H), 8.22 (s, 1H), 7.60 (d, 1H), 7.42 (d, 1H), 6.91 (s, 1H), 3.95 (s, 3H).

### Example 95

### 5-Thiophen-3-yl-isoxazole-3-carboxylic acid methyl ester

A solution of 2,4-dioxo-4-thiophen-3-yl-butyric acid methyl ester (4.0 g, 18.8 mmol) and hydroxylamine hydrochloride (3.9 g, 56.5 mmol) in methanol (150 mL) was refluxed at 80° C. for 1 hour. After cooling, the mixture was diluted with ethyl acetate, washed with water and saturated brine, dried over anhydrous sodium sulfate, filtered and concentrated. The resulting residue was purified by flash column chromatography using 15-30% ethyl acetate in hexane to afford 5-thiophen-3-yl-isoxazole-3-carboxylic acid methyl ester (3.37 g, 86%, white solid). ¹H NMR (CDCl₃) δ (ppm): 7.88 (s, 1H), 7.46 (m, 2H), 6.81 (s, 1H), 4.02 (s, 3H).

### Example 96

### 5-(Thiophen-3-yl-isoxazol-3-yl)methanol

Lithium aluminum hydride (363 mg, 9.6 mmol) was added in 3 portions to a solution of 5-thiophen-3-yl-isoxazole-3-carboxylic acid methyl ester (2.0 g, 9.6 mmol) in THF (100 mL) in an ice-bath. The mixture was warmed to room temperature and stirred for 1 hour. After quenching the reaction with ice and then diluting with ethyl acetate, the organic layer was washed with saturated brine, dried over anhydrous sodium sulfate, filtered and concentrated to afford the titled compound (1.72 g, 99%, white solid). ¹H NMR (CDCl₃) δ (ppm): 7.80 (m, 1H), 7.43 (m, 2H), 6.47 (m, 1H), 4.82 (s, 2H), 2.19 (bs, 1H).

### Example 97

### Methanesulfonic acid 5-thiophen-3-yl-isoxazol-3-ylmethyl ester

Triethyl amine (2.63 mL, 19.0 mmol) and methanesulfonyl chloride (1.1 mL, 14.2 mmol) were added to a solution of 5-(thiophen-3-yl-isoxazol-3-yl)methanol (1.72 mg, 9.5 mmol) in dichloromethane (100 mL) at 0° C. After 1 hour, the reaction mixture was quenched with cold saturated sodium bicarbonate and then the organic layer was washed with saturated brine, dried over anhydrous sodium sulfate, filtered, and concentrated to afford the titled compound (2.46 mg, 99%). ¹H NMR (CDCl) δ (ppm): 7.84 (m, 1H), 7.45 (m, 2H), 6.56 (s, 1H), 5.36 (s, 2H), 3.11 (s, 3H).

### Example 98

### Benzoic acid 2-nitro-ethyl ester

To a benzene solution (40 mL) of 2-nitro-ethanol (4.55 g, 50 mL), benzoyl chloride (7.03 g, 50 mmol) was added at room temperature. The reaction mixture was heated at 800° C. for 24 hours. The mixture was concentrated and the residue was purified by column chromatography with ether: hexanes (1:1) to give 6.76 g of benzoic acid 2-nitro-ethyl ester as white solid. ¹H-NMR(CDCl3): δ(ppm): 8.03 (d, 2H), 7.61 (t, 1H), 7.47 (t, 2H), 4.88(m, 2H) and 4.77 (m, 2H).

### Example 99

### 4-(2-Nitro-ethyl)-piperazine-1-carboxylic acid ethyl ester

To an ethanol solution (60 mL) of benzoic acid 2-nitro-ethyl ester (1.95 g, 10 mmol), piperazine-1-carboxylic acid ethyl ester (1.58 g, 10 mmol) was added at room temperature. After being stirred for 2 hours, the reaction mixture was concentrated. The residue was mixed with ether and saturated sodium bicarbonate. The organic layer was dried with Mg SO4, concentrated to give 1.95 g (84.3%) of 4-(2-nitro-ethyl)-piperazine-1-carboxylic acid ethyl ester as clear oil. ¹H-NMR(CDCl₃): δ(ppm): 4.52 (t, 2H), 4.15 (q, 2H), 3.48 (m, 4H), 3.04 (t, 2H), 2.50 (m, 4H) and 1.27 (t, 3H).

### Example 100

### 4-(1-Methyl-2-nitro-ethyl)-piperazine-1-carboxylic acid ethyl ester

To a mixed THF (30 ml) and ethanol (10 mL) solution of piperazine-1-carboxylic acid ethyl ester (4.75 g, 30 mmol) and nitromethane (2.75 g, 45 mmol), acetaldehyde (1.32 g, 30 mmol) was added and followed by the addition of KOt-Bu (3 mL, 1 M). The reaction mixture was stirred overnight. Standard work-up. The product was purified by column chromatography with 20˜30% of ethyl acetate in hexanes to give 2.27 g (30.7%) of 4-(1-methyl-2-nitro-ethyl)-piperazine-1-carboxylic acid ethyl ester as yellow oil. ¹H-NMR(CDCl₃): δ(ppm): 4.50 (m, 1H), 4.26 (dd, 1H), 4.13 (q, 2H), 4.50(m, 5H), 2.58 (m, 2H), 2.45 (m, 2H), 1.28 (t, 3H) and 1.08 (d, 3H).

### Example 101

### 4-(5-Tributylstannanyl-isoxazol-3-ylmethyl)-piperazine-1-carboxylic acid ethyl ester

Ethynyl-tributyl-stannane (5.0 g, 16.1 mmol) was added to a benzene solution (90 mL) of 4-(2-nitro-ethyl)-piperazine-1-carboxylic acid ethyl ester (2.31 g, 10 mmol) and PhNCO (3.57 g, 30 mmol) under argon, and followed by the addition of triethylamine (1 mL). The reaction mixture was stirred at room temperature overnight, then filtered and washed with hexanes. The filtrate was concentrated and triturated with hexanes again. The hexanes solution was concentrated, purified by column chromatography with 20% ethyl acetate in hexanes. The elusion was concentrated and triturated with hexanes. The filtrate was concentrated again to give 5.1 g (96%) of 4-(5-tributylstannanyl-isoxazol-3-ylmethyl)-piperazine-1-carboxylic acid ethyl ester as yellow oil. ¹H-NMR(CDCl₃) δ(ppm): 6.40 (s, 1H), 4.14 (q, 2H), 3.69 (s, 2H), 3.51(m, 4H), 2.48 (m, 4H), 1.05-1.70 (m, 21H) and 0.91 (t, 9H).

### Example 102

### 4-[1-(5-Tributylstannanyl-isoxazol-3-yl)-ethyl]-piperazine-1-carboxylic acid ethyl ester

4-[1-(5-Tributylstannanyl-isoxazol-3-yl)-ethyl]-piperazine-1-carboxylic acid ethyl ester (3.2 g, 64.1%) as yellow oil was obtained from 4-(1-methyl-2-nitro-ethyl)-piperazine-1-carboxylic acid ethyl ester (2.27 g, 9.2 mmol) reacted with ethynyl-tributyl-stannane (5.0 g, 16.1 mmol), PhNCO (3.57 g, 30 mmol) and triethylamine (1 mL) in benzene. ¹H-NMR(CDCl₃) δ(ppm): 6.33 (s, 1H), 4.12 (q, 2H), 3.92 (m, 1H), 3.49 (m, 4H), 2.47 (m, 4H), 1.05-1.70 (m, 24H) and 0.90 (t, 9H).

### Example 103

### 1,1,1-Trifluoro-3-nitro-propan-2-ol

1-Ethoxy-2,2,2-trifluoro-ethanol (7.62 g, 52.9 mmol) was mixed with nitromethane (3.26 g, 52.9 mmol) and K2CO3 (7.3 g, 52.9 mmol) in dichloromethane (5 mL) and ethanol (10 m L) for 3 days the reaction mixture was quenched with saturated NH4Cl and extracted with ether. The organic layer was dried with MgSO4 and concentrated to give 7.2 g (85%) of 1,1,1-trifluoro-3-nitro-propan-2-ol as pale-brown oil. ¹H-NMR(CDCl₃): δ(ppm): 4.88 (m, 1H), 4.65 (m, 2H) and 3.66 (d, 1H).

### Example 104

### 4-(2,2,2-Trifluoro-1-nitromethyl-ethyl)-piperazine-1-carboxylic acid ethyl ester

1,1,1-trifluoro-3-nitro-propan-2-ol (2.46 g, 15.5 mmol) was mixed with acetyl chloride (1.36 g, 17.3 mmol) at 30˜35° C. for 3 days. The reaction mixture was quenched with ethanol (20 mL), followed by the addition of piperazine-1-carboxylic acid ethyl ester (2.45 g, 15.5 mmol) and stirred at room temperature for an hour. Dichlormethane was added to the reaction mixture and washed with water and brine. The organic layer was dried with MgSO4 and concentrated. The residue was triturated with hexanes to give 3.3 g (71.1%) of 4-(2,2,2-trifluoro-1-nitromethyl-ethyl)-piperazine-1-carboxylic acid ethyl ester. 1H-NMR(CDCl₃) δ(ppm): 4.67 (dd, 1H), 4.57 (dd, 1H), 4.13 (m, 3H), 3.43 (m, 4H), 2.95 (m, 2H), 2.68 (m, 2H) and 1.27 (t, 3H).

### Example 105

### 5-(3-Chloro-phenyl)-2-methyl-oxazole

To a solution of Tl(OAc)₃ (4.2 g, 11.1 mmol) in acetonitrile (80 mL), trifluoromethanesulfuric acid (5 g, 33.3 mmol) was added dropwise at room temperature and stirred for 15 minutes. The reaction mixture was then heated to 80° C. and 1-(3-chloro-phenyl)-ethanone (1.14 g, 7.4 mmol) in acetonitrile (40 mL) was added. After one hour, the reaction was quenched with dichloromethane and saturated sodium bicarbonate. The organic layer was dried, purified by column chromatography with 5˜19% ethyl acetate in hexanes to give 1.2 (83.9%) g of 5-(3-chloro-phenyl)-2-methyl-oxazole as yellow oil. ¹H-NMR(CDCl₃) δ(ppm): 7.60 (s, 1H), 7.48 (d, 1H), 7.29 (m, 2H), 7.23 (s, 1H) and 2.34 (s, 3H).

### Example 106

### 2-Bromomethyl-5-(3-chloro-phenyl)-oxazole

5-(3-chloro-phenyl)-2-methyl-oxazole (580 mg, 3 mmol) was mixed with NBS (531 mg, 3 mmol) and BPO (36.3 mg, 0.15 mmol) in CCl4 at room temperature. The reaction mixture was heated at 75° C. for 2 hours and then quenched with water and dichloromethane. The organic layer was dried, concentrated, purified by column chromatography with 2˜5% ethyl acetate in hexanes to give 562 mg (68.3%) of 2-bromomethyl-5-(3-chloro-phenyl)-oxazole as yellow oil. ¹H-NMR(CDCl₃) δ(ppm): 7.67 (s, 1H), 7.54 (d, 1H), 7.35 (m, 3H) and 4.56 (s, 2H).

### Example 107

### 4-(Cyano-[5-(2-fluoro-5-methyl-phenyl)-isoxazol-3-yl]-methyl}-piperazine-1-carboxylic acid ethyl ester

In a screw cap vial equipped with stir bar added 5-(2-fluoro-5-methyl-phenyl)-isoxazole-3-carbaldehyde (50 mg, 0.24 mmol), and tetrahydrofuran (2 ml). To this solution added piperazine-1-carboxylic acid ethyl ester (0.16 ml, 1.1 mmol) followed by diethylcyanophosphonate (0.08 ml, 0.60 mmol). Reaction mixture was concentrated in-vacuo. The residue was dissolved in dichloromethane (50 ml), successively washed with water (50 ml), saturated sodium carbonate (aqueous, 50 ml), water (50 ml) and brine (50 ml). The organic phase was dried (sodium sulfate), filtered and concentrated in-vacuo. The crude residue was purified on silica gel using 2% ethyl acetate in dichloromethane to isolate an off-white solid. The isolated solid was triturated with mixture of hexanes and ethyl acetate to isolate the title compound as a white solid (48 mg, 54%). ¹H-NMR (CDCl₃), δ (ppm): 7.76 (dd, 1H), 7.25 (m, 1H), 7.10 (m, 1H), 6.80 (d, 1H), 4.98 (s, 1H), 4.15 (q, 2H), 3.58 (m, 4H), 2.67 (m, 4H), 2.42 (s, 3H), 1.28 (t, 3H).

### Example 108

### 4-[5-(3-Chloro-phenyl)-[1,2,4]oxadiazol-3-ylmethyl]-2-oxo-piperazine-1-carboxylic acid ethyl ester

Piperazinone (131 mg, 1.31 mmol) was added to a mixture of 3-Chloromethyl-5-(3-chloro-phenyl)-[1,2,4]oxadiazole (200 mg, 0.87 mmol) and potassium carbonate (362 mg, 2.62 mmol) in acetonitrile (1 mL) and the resulting mixture was stirred at room temperature overnight. The reaction mixture was diluted with ethyl acetate, washed with water and brine, dried over anhydrous sodium sulfate, filtered, and concentrated. The product was obtained by solid phase extraction chromatography (SPE) on silica gel using ethyl acetate-hexanes as eluant giving 4-[5-(3-Chloro-phenyl)-[1,2,4]oxadiazol-3-ylmethyl]-piperazin-2-one (62 mg, 24% yield) as a white solid. 1H NMR (CDCl₃) δ (ppm): 8.18 (s, 1H), 8.05 (dd, 1H), 7.60 (dd, 1H), 7.49 (t, 1H), 6.69 (br, s, 1H), 3.88 (s, 2H), 3.43 (m, 2H), 3.38 (s, 2H), 2.86 (t, 2H).

To a solution 4-[5-(3-Chloro-phenyl)-[1,2,4]oxadiazol-3-ylmethyl]-piperazin-2-one (50 mg, 0.17 mmol) in THF (5 ml) at −78° C. was added n-BuLi (0.1 ml, 1.6 M sol'n in Hexane, 0.16 mmol) and the mixture was stirred at this temperature for 15 minutes. Ethylchloroformate was then added and the resulting mixture was stirred for a further 15 minutes before quenching with saturated NH4Cl. The mixture was then extracted with ethyl acetate (2×15 ml) and the combined organic extract was then washed with brine and then dried over MgSO4 (anhydrous). The solvent was then removed in vacuo and the residue purified by flash chromatography giving 28 mg (45% yield) as a white solid. 1H NMR (CDCl₃) δ (ppm): 8.18 (t, 1H), 8.05 (dd, 1H), 7.59 (dd, 1H), 7.48 (t, 1H), 4.34 (q, 2H), 3.87 (s, 2H), 3.82 (dd, 2H), 3.52 (s, 2H), 2.94 (dd, 3H), 1.29 (t, 3H).

### Example 109

### 4-[1-(5-m-Tolyl-1,2,4]oxadiazol-3-yl)-ethyl]-piperazine-1-carboxylic acid ethyl-methyl-amide

To a solution of N-Boc-piperazine (5.0 g, 26.8 mmol) in CH₂Cl₂ was added, Et₃N (3.74 ml, 26. mmol) followed by carbonyldiimidazole (4.35 g, 26.8 mmol) and the mixture was stirred overnight. The solvent was then removed in vacuo, the residue diluted with CH2Cl2 (60 ml), washed with water (2×50 ml), then with brine and the organic layer was dried over Na2SO4 (anhydrous). Removal of the solvent in vacuo gave 6.4 g of a white solid which was dissolved in acetonitrile (30 ml) and then treated with MeI (12.6 g, 88.5 mmol) and the mixture was stirred overnight. The solvent was removed in vacuo and the crude product (8.1 g, 71% yield, white solid) was used without further purification. To the crude product (300 mg, 0.7 mmol), Et3N (0.5 ml, 3.5 mol) in CH2Cl2 was added N-ethyl-N-methylamine (207 mg, 3.4 mmol) and the mixture was stirred at room temperature overnight. The reaction mixture was diluted with ether and then extracted with water. The organic extract was then dried over Na2SO4 (anhydrous) and the solvent removed in vacuo to afford the crude residue that was immediately treated with TFA/CH2Cl2 (1:1) for 1 h. The mixture was the poured into saturated NaHCO3 followed by extraction with CH2Cl2. Subsequent washing and drying of the organic layer along with removal of the solvent in vacuo afforded the piperazine-1-carboxylic acid ethyl-methyl-amide (20 mg, 17% yield) as a colourless oil.
4-[1-(5-m-Tolyl-[1,2,4]oxadiazol-3-yl)-ethyl]-piperazine-1-carboxylic acid ethyl-methyl-amide (5.3 mg, 13% yield, white semi-solid) obtained from 3-Chloromethyl-5-m-tolyl-[1,2,4]oxadiazole (70 mg, 0.34 mmol), K₂CO₃ (93 mg, 0.67 mmol) and piperazine-1-carboxylic acid ethyl-methyl-amide (20 mg, 0.17 mmol) in acetonitrile ¹H-NMR (CDCl₃), δ (ppm): 7.98 (m, 2H), 7.43 (m, 2H), 3.80 (s, 2H), 3.31 (t, 4H), 3.22 (q, 2H), 3.13 (m, 1H), 2.81 (s, 3H), 2.64 (t, 4H), 2.46 (s, 3H), 1.15 (t, 3H).

### Example 110

### (R)-and (S)-4-1-(5-(3-Methyl-phenyl)-[1,2,4]oxadiazol-3-yl)-ethyl]-piperazine-carboxylic acid ethyl ester

(R)-4-[1-(5-(3-Methyl-phenyl)-[1,2,4]oxadiazol-3-yl)-ethyl]-piperazine-carboxylic acid ethyl ester (72 mg, colorless oil, 80% yield) was prepared from (R)-1-[1-(5-(3-methyl-phenyl)-[1,2,4]oxadiazol-3-yl)-ethyl]-piperazine (70 mg, 0.26 mmol).
(S)-4-[1-(5-(3-Methyl-phenyl)-[1,2,4]oxadiazol-3-yl)-ethyl]-piperazine-carboxylic acid ethyl ester (62 mg, colorless oil, 72% yield) was prepared from (S)1-[1-(5-(3-methyl-phenyl)-[1,2,4]oxadiazol-3-yl)-ethyl]-piperazine (70 mg, 0.25 mmol)

### Example 111

### (R)-and (S)-4-[1-(5-(3-Methyl-phenyl)-[1,2,4]oxadiazol-3-yl)-ethyl]-piperazine-carboxylic acid ethyl ester

(R)-4-[1-(5-(3-Methyl-phenyl)-[1,2,4]oxadiazol-3-yl)-ethyl]-piperazine-carboxylic acid ethyl ester (72 mg, colorless oil, 80% yield) was prepared from (R)-1-[1-(5-(3-methyl-phenyl)-[1,2,4]oxadiazol-3-yl)-ethyl]-piperazine (70 mg, 0.26 mmol).
(S)-4-[1-(5-(3-Methyl-phenyl)-[1,2,4]oxadiazol-3-yl)-ethyl]-piperazine-carboxylic acid ethyl ester(62 mg, colorless oil, 72% yield) was prepared from (S)1-[1-(5-(3-methyl-phenyl)-[1,2,4]oxadiazol-3-yl)-ethyl]-piperazine (70 mg, 0.25 mmol)

### Example 112

### 4-{1-[5-(3-Chloro-phenyl)-[1,2,4]oxadiazol-3-yl]-propyl}-piperazine-1-carboxylic acid ethyl ester

4-{1-[5-(3-Chloro-phenyl)-[1,2,4]oxadiazol-3-yl]-propyl}-piperazine-1-carboxylic acid ethyl ester (33 mg, 87% yield) obtained from 1-{1-[5-(3-Chloro-phenyl)-[1,2,4]oxadiazol-3-yl]-propyl}-piperazine (32 mg, 0.1 mmol). I H NMR (CDCl₃) δ (ppm): 8.16 (t, 1H), 8.03 (dd, 1H), 7.58 (dd, 1H), 7.50 (t, 1H), 4.10 (q, 2H), 3.80 (dd, 1H), 3.49 (m, 4H), 2.56 (m, 4H), 2.04 (m, 2H), 1.24 (t, 3H), 0.95 (t, 3H).

### Example 113

### (S)-4-{1-[5-(5-Chloro-2-fluoro-phenyl)-[1,2,4]oxadiazol-3-yl]-ethyl}-piperazine-1-carboxylic acid ethyl ester

4-{1-[5-(5-Chloro-2-fluoro-phenyl)-[1,2,4]oxadiazol-3-yl]-ethyl}-piperazine-1-carboxylic acid ethyl ester (3.4 mg, 28% yield, semi-solid) obtained from 4-{1-[5-(5-Chloro-2-fluoro-phenyl-[1,2,4]oxadiazol-3-yl]-propyl}-piperazine (10 mg, 0.032 mmol).

¹H NMR (CDCl₃) δ (ppm): 8.16 (dd, 1H), 7.56 (m, 1H), 7.24 (t, 1H), 4.12 (q, 2H), 4.08 (q, 1H), 3.52 (m, 4H), 2.57 (m, 4H), 1.57 (d, 3H), 1.26 (t, 3H).

### Example 114

### (S)-{1-[5-(2-Fluoro-5-methyl-phenyl)-[1,2,4]oxadiazol-3-yl]-ethyl}-piperazine-1-carboxylic acid ethyl ester

The title compound (82 mg, 73% yield, colouress oil) was obtained from 1-{1-[5-(2-Fluoro-5-methyl-phenyl)-[1,2,4]oxadiazol-3-yl]-ethyl}-piperazine (91 mg, 0.31 mmol) ¹H NMR (CDCl₃) δ (ppm): 7.94 (dd, 1H), 7.37 (m, 1H), 7.16 (dd, 1H), 4.10 (q, 2H), 4.07 (q, 1H), 3.52 (m, 4H), 2.60 (m, 4H), 2.42 (s, 3H), 1.57 (d, 3H), 1.25 (t, 3H).

### Example 115

### (S)-4-{1-[5-(3-Chloro-phenyl)-[1,2,4]oxadiazol-3-yl]-ethyl}-piperazine-1-carboxylic acid ethyl ester

The title compound (40 mg, 73% yield, colourless oil) was obtained from 1-{1-[5-(3-Chloro-phenyl)-[1,2,4]oxadiazol-3-yl]-ethyl}-piperazine (43 mg, 0.15 mmol)

### Example 116

### (R)-4-[5-(2-Fluoro-5-methyl-phenyl)-[1,2,4]oxadiazol-3-ylmethyl]-2-methyl-piperazine-1-carboxylic acid ethyl ester

The title compound (28 mg, 66%, colourless oil) was obtained from 1-(5-m-tolyl-[1,2,4]oxadiazol-3-yl-(R)-methyl)-piperazine (34.6 mg, 0.12 mmol), dichloromethane (2 mL) and triethylamine (49 μl, 0.36 mmol) with methyl chloroformate (21 μl, 0.24 mmol) in ice bath at room temperature for ½ h. Purification was performed on silica gel using 10-20% ethyl acetate in hexanes. ¹H-NMR (CDCl₃), δ (ppm): 7.94 (dd, 1H), 7.39 (m, 1H), 7.16 (q, 1H), 4.32 (m, 1H), 4.13 (m, 2H), 3.81 (m, 3H), 3.23 (dt, 1H), 2.97 (d, 1H), 2.94 (d, 1H), 2.76 (d, 1H), 2.40 (d, 1H), 2.37 (dt, 1H), 1.27 (m, 6H).

### Example 117

### (S)-4-[5-(2-Fluoro-5-methyl-phenyl)-[1,2,4]oxadiazol-3-ylmethyl]-2-methyl-piperazine-1-carboxylic acid ethyl ester

The title compound (40 mg, 83%, colourless oil) was obtained from 1-(5-m-tolyl-[1,2,4]oxadiazol-3-yl-(S)-methyl)-piperazine (38.3 mg, 0.13 mmol), dichloromethane (2 mL) and triethylamine (55 μl, 0.40 mmol) with methyl chloroformate (25 μl, 0.26 mmol) in ice bath at room temperature for ½ h. Purification was performed on silica gel using 15-25% ethyl acetate in hexanes. ¹H-NMR (CDCl₃), δ (ppm): 7.93 (d, 1H), 7.39 (m, 1H), 7.15 (q, 1H), 4.32 (m, 1H), 4.13 (m, 2H), 3.82 (m, 3H), 3.22 (dt, 1H), 2.93 (d, 1H), 2.76 (d, 1H), 2.40 (m, 4H), 2.37 (dt, 1H), 1.27 (m, 6H).

### Example 118

### (R)-3-Methyl-4-(5-m-tolyl-[1,2,4]oxadiazol-3-ylmethyl)-piperazine-1-carboxylic acid ethyl ester and (S)-3-Methyl-4-(5-m-tolyl-[1,2,4]oxadiazol-3-ylmethyl)-piperazine-1-carboxylic acid ethyl ester (R)-3-Methyl-4-(5-m-tolyl-[1,2,4]oxadiazol-3-ylmethyl)-piperazine-1-carboxylic acid ethyl ester (80 mg, 96% yield, colourless oil) and (S)-3-Methyl-4-(5-m-tolyl-[1,2,4]oxadiazol-3-ylmethyl)-piperazine-1-carboxylic acid ethyl ester (81 mg, 98% yield, colourless oil) obtained from 3-chloromethyl-5-m-tolyl-[1,2,4]oxadiazole (50 mg, 0.24 mmol), K₂CO₃ (100 mg, 0.72 mmol) and (R)- or (S)-3-methyl-piperazine-1-carboxylic acid ethyl ester (83 mg, 0.48 mmol) in acetonitrile: both R and S-isomers: ¹H-NMR (CDCl₃), δ (ppm): 7.93 (m, 2H), 7.40 (m, 2H), 4.12 (q, 2H), 4.02 (s, 2H), 3.91 (m, 2H), 3.13 (m, 1H), 2.86 (m, 2H), 2.54 (m, 2H), 2.45 (s, 3H), 1.24 (t, 3H), 1.21 (d, 3H).

### Example 119

### 4-[5-(3-Methylsulfanyl-phenyl)-[1,2,4]oxadiazol-3-ylmethyl]-piperazine-1-carboxylic acid ethyl ester

The title compound (62 mg, 81%, colorless oil) was obtained from 3-chloromethyl-5-(3-methylsulfanyl-phenyl)-[1,2,4]oxadiazole (50 mg, 0.21 mmol), potassium carbonate (86.1 mg, 0.62 mmol), and piperazine-1-carboxylic acid ethyl ester (65.7 mg, 0.42 mmol) in acetonitrile (2 mL). Purification was performed by SPE (flash) chromatography using 40% ethyl acetate in hexanes. ¹H NMR (CDCl₃) δ (ppm): 8.01 (s, 1H), 7.91 (d, 1H), 7.43 (m, 2H), 4.13 (q, 2H), 3.79 (s, 2H), 3.59 (t, 4H), 2.59 (t, 4H), 2.56 (s, 3H), 1.26 (t, 3H).

### Example 120

### 4-[5-(2-Fluoro-5-methyl-phenyl)-[1,2,4]oxadiazol-3-ylmethyl]-piperazine-1-carboxylic acid ethyl ester

The title compound (45.6 mg, 99.1%) was obtained from piperazine-1-carboxylic acid ethyl ester (23.2 μL, 0.158 mmol), 3-chloromethyl-5-(2-fluoro-5-methyl-phenyl)-[1,2,4]oxadiazole (30 mg, 0.132 mmol), and K₂CO₃ (45.3 mg, 0.328 mmol) in acetonitrile (0.5 mL) at room temperature overnight. Purification was performed by SPE chromatography on silica gel with 20-40% ethyl acetate in hexanes. ¹H-NMR (CDCl₃), δ (ppm): 7.95 (dd, 1H), 7.37 (m, 1H), 7.15 (t, 1H), 4.13 (q, 2H), 3.82 (s, 2H), 3.54 (t, 4H), 2.60 (t, 4H), 2.41 (s, 3H), 1.26 (t, 3H).

### Example 121

### 4-[5-(3-Chloro-phenyl)-isoxazol-3-ylmethyl]-piperazine-1-carboxylic acid ethyl ester

The title compound (66.9 mg, 91%, colorless oil) was obtained from methanesulfonic acid 5-(3-chloro-phenyl)-isoxazol-3-ylmethyl ester (60 mg, 0.21 mmol), potassium carbonate (86.5 mg, 0.63 mmol), and piperazine-1-carboxylic acid ethyl ester (0.0616 mL, 0.42 mmol) in acetonitrile (2 mL). Purification was performed by SPE (flash) chromatography using 40-60% ethyl acetate in hexanes. ¹H NMR (CDCl₃) δ (ppm): 7.78 (m, 1H), 7.69 (m, 1H), 7.43 (m, 2H), 6.61 (s, 1H), 4.15 (q, 2H), 3.67 (s, 2H), 3.53 (t, 4H), 2.51 (t, 4H), 1.28 (t, 3H).

### Example 122

### 4-[5-(2-Fluoro-5-methyl-phenyl)-[1,2,4]oxadiazol-3-yl-(R)-methyl]-3-methyl-piperazine-1-carboxylic acid ethyl ester

The title compound (37.1 mg, 77.6%) was obtained from (R)-3-methyl-piperazine-1-carboxylic acid ethyl ester (27.2 mg, 0.158 mmol), 3-chloromethyl-5-(2-fluoro-5-methyl-phenyl)-[1,2,4]oxadiazole (30 mg, 0.132 mmol), and K₂CO₃ (45.3 mg, 0.328 mmol) in acetonitrile (0.5+1.0 mL) at room temperature overnight. Purification was performed by SPE chromatography on silica gel with 100 mL 20%, 100 mL 30%, 50 mL 35% ethyl acetate in hexanes. ¹H-NMR (CDCl₃), δ (ppm): 7.93 (dd, 1H), 7.37 (m, 1H), 7.16 (q, 1H), 4.12 (q, 2H), 4.02 (s, 2H), 3.91 (bs, 2H), 3.16 (dt, 1H), 2.89 (m, 2H), 2.59 (m, 2H), 2.416 (s, 3H), 1.24 (m, 5H).

### Example 123

### 4-[5-(2-Fluoro-5-methyl-phenyl)-[1,2,4]oxadiazol-3-yl-(S)-methyl]-3-methyl-piperazine-1-carboxylic acid ethyl ester

The title compound (40.1 mg, 83.9%) was obtained from (S)-3-methyl-piperazine-1-carboxylic acid ethyl ester (27.2 mg, 0.158 mmol), 3-chloromethyl-5-(2-fluoro-5-methyl-phenyl)-[1,2,4]oxadiazole (30 mg, 0.132 mmol), and K₂CO₃ (45.3 mg, 0.328 mmol) in acetonitrile (0.5 mL) at room temperature overnight. Purification was performed by SPE chromatography on silica gel with 20-35% ethyl acetate in hexanes. ¹H-NMR (CDCl₃), δ (ppm): 7.94 (dd, 1H), 7.38 (m, 1H), 7.16 (q, 11H), 4.13 (m, 2H), 4.02 (s, 2H), 3.89 (bs, 2H), 3.14 (dt, 1H), 2.88 (m, 2H), 2.57 (m, 2H), 2.42 (d, 3H), 1.26 (m, 5H).

### Example 124

### 4-[5-(5-Bromo-2-fluoro-phenyl)-[1,2,4]oxadiazol-3-ylmethyl]-piperazine-1-carboxylic acid ethyl ester

The title compound (61.2 mg, 86.1%) was obtained from piperazine-1-carboxylic acid ethyl ester (29.6 μL, 0.202 mmol), 5-(5-Bromo-2-fluoro-phenyl)-3-chloromethyl-[1,2,4]oxadiazole (50 mg, 0.172 mmol), and K₂CO₃ (72.9 mg, 0.528 mmol) in acetonitrile (0.5 mL) at room temperature overnight. Purification was performed by SPE chromatography on silica gel with 20-30% ethyl acetate in hexanes. ¹H-NMR (CDCl₃), δ (ppm): 8.32 (dd, 1H), 7.70 (m, 1H), 7.18 (q, 1H), 4.13 (m, 2H), 3.82 (s, 2H), 3.54 (t, 4H), 2.60 (t, 4H), 1.26 (q, 3H).

### Example 125

### 4-[5-(2,5-Dichloro-phenyl)-[1,2,4]oxadiazol-3-ylmethyl]-piperazine-1-carboxylic acid ethyl ester

The title compound (57.2 mg, 78.1%) was obtained from piperazine-1-carboxylic acid ethyl ester (33.1 μL, 0.226 mmol), 3-chloromethyl-5-(2,5-dichloro-phenyl)-[1,2,4]oxadiazole (50 mg, 0.189 mmol), and K₂CO₃ (65 mg, 0.47 mmol) in acetonitrile (0.75 mL) at room temperature overnight. Purification was performed by SPE chromatography on silica gel with 50% ethyl acetate in hexanes. ¹H-NMR (CDCl₃), δ (ppm): 8.13 (m, 1H), 7.50 (m, 2H), 4.14 (m, 2H), 3.84 (s, 2H), 3.56 (t, 4H), 2.62 (t, 4H), 1.28 (q, 3H).

### Example 126

### 4-(5-Thiophen-3-yl-isoxazol-3-ylmethyl)-piperazine-1-carboxylic acid ethyl ester

The title compound (59.4 mg, 97%, colorless oil) was obtained from methanesulfonic acid 5-thiophen-3-yl-isoxazol-3-ylmethyl ester (50 mg, 0.19 mmol), potassium carbonate (80 mg, 0.58 mmol), and piperazine-1-carboxylic acid ethyl ester (0.0565 mL, 0.39 mmol) in acetonitrile (2 mL). Purification was performed by SPE (flash) chromatography using 40% ethyl acetate in hexanes. 1H NMR (CDCl₃) δ (ppm): 7.80 (m, 1H), 7.43 (m, 2H), 6.43 (s, 1H), 4.15 (q, 2), 3.66 (s, 2H), 3.52 (t, 4H), 2.51 (t, 4H), 1.28 (t, 3H).

### Example 127

### 4-[5-(2-Fluoro-5-methyl-phenyl)-isoxazol-3-ylmethyl]-piperazine-1-carboxylic acid ethyl ester

The title compound (36.0 mg, 60%, white solid) was obtained from methanesulfonic acid 5-(2-fluoro-5-methyl-phenyl)-isoxazol-3-ylmethyl ester (50 mg, 0.174 mmol), potassium carbonate (72 mg, 0.521 mmol), and piperazine-1-carboxylic acid ethyl ester (0.0509 mL, 0.348 mmol) in acetonitrile (2 mL). Purification was performed by SPE (flash) chromatography using 40-60% ethyl acetate in hexanes. ¹H NMR (CDCl₃) δ (ppm): 7.76 (m, 1H), 7.22 (m, 1H), 7.09 (m, 1H), 6.73 (d, 1H), 4.15 (q, 2H), 3.69 (s, 2H), 3.53 (t, 4H), 2.52 (t, 4H), 2.41 (s, 3H), 1.27 (t, 3H).

### Example 128

### 4-{1-[5-(3-Chloro-phenyl)-isoxazol-3-yl]-ethyl}-piperazine-1-carboxylic acid ethyl ester

The title compound (37 mg, white solid) was obtained from methanesulfonic acid 1-[5-(3-chloro-phenyl)-isoxazol-3-yl]-ethyl ester (49.3 mg, 0.16 mmol), potassium carbonate (113 mg, 0.82 mmol) and piperazine-1-carboxylic acid ethyl ester (0.05 ml, 0.33 mmol) in acetonitrile (2 ml) at 80° C. overnight. Reaction mixture was filtered and filtrate was concentrated in-vacuo. The crude residue was purified on silica gel using 30% ethyl acetate in hexanes. ¹H-NMR (CDCl₃), δ (ppm): 7.78 (m, 1H), 7.77 (m, 1H), 7.43 (m, 2H), 6.54 (s, 1H), 4.12 (q, 2H), 3.88 (q, 1H), 3.50 (m, 4H), 2.52 (m, 4H), 1.45 (d, 3H), 1.27 (t, 3H).

### Example 129

### 4-{1-[5-(2-Fluoro-5-methyl-phenyl)-isoxazol-3-yl]-ethyl}-piperazine-1-carboxylic acid ethyl ester

The title compound (1.08 g, yellow oil) was obtained from methanesulfonic acid 1-[5-(2-fluoro-5-methyl-phenyl)-isoxazol-3-yl]-ethyl ester (853 mg, 3.86 mmol), potassium carbonate (2.6 g, 19.3 mmol) and piperazine-1-carboxylic acid ethyl ester (2.66 ml, 15.4 mmol) in acetonitrile (15 ml) at 80° C. overnight. Reaction mixture was cooled to room temperature, diluted with ethyl acetate (50 ml), sequentially washed with water (50 ml) and brine (50 ml), dried (sodium sulfate), filtered and concentrated, in-vacuo. The crude residue was purified on silica gel using 10% ethyl acetate in hexanes. ¹H-NMR (CDCl₃), δ (ppm): 7.74 (dd, 1H), 7.19 (m, 1H), 7.06 (m, 1H), 6.63 (d, 1H), 4.13 (q, 2H), 3.90 (q, 1H), 3.48 (m, 4H), 2.51 (m, 4H), 2.39 (s, 3H), 1.48 (d, 3H), 1.24 (t, 3H).

The isolated free base was dissolved in methanol (10 ml) and treated with hydrochloric acid (1N in diethyl ether, 6 ml). The reaction mixture was stirred at room temperature for 20 minutes and concentrated in-vacuo. The isolated salt was washed with diethyl ether to isolate hydrochloride salt of the title compound as white solid (0.83 g).

### Example 130

### (R)- and (S)-4-{1-[5-(2-Fluoro-5-methyl-phenyl)-isoxazol-3-yl]-ethyl}-piperazine-1-carboxylic acid ethyl ester enantiomers

The product above was separated by chiral column Chiracel OD with isopropanol (0.5% Et2NH): hexanes (5:95) to give two enantiomers Rt=7.74 min & 9.69 min respectively.

### Example 131

### 4-{1-[5-(2-Fluoro-5-methyl-phenyl)-isoxazol-3-yl]-propyl}-piperazine-1-carboxylic acid ethyl ester

The title compound (8 mg, clear oil) was obtained from methanesulfonic acid 1-[5-(2-fluoro-5-methyl-phenyl)-isoxazol-3-yl]-propyl ester (50 mg, 0.16 mmol), potassium carbonate (109 mg, 0.79 mmol) and piperazine-1-carboxylic acid ethyl ester (0.05 ml, 0.32 mmol) in acetonitrile (2 ml) at 80° C. overnight. Reaction mixture was cooled to room temperature, diluted with ethyl acetate (10 ml), sequentially washed with water (10 ml) and brine (10 ml), dried (sodium sulfate), filtered and concentrated, in-vacuo. The crude residue was purified on silica gel using 10% ethyl acetate in hexanes. ¹H-NMR (CDCl₃), δ (ppm): 7.77 (dd, 1H), 7.19 (m, 1H), 7.08 (m, 1H), 6.57 (d, 1H), 4.13 (q, 2H), 3.69 (q, 1H), 3.48 (m, 4H), 2.48 (m, 4H), 2.40 (s, 3H), 1.92 (m, 2H), 1.27 (t, 3H), 0.92 (t, 3H).

### Example 132

### 4-{Cyclopropyl-[5-(2-fluoro-5-methyl-phenyl)-isoxazol-3-yl]-methyl}-piperazine-1-carboxylic acid ethyl ester

The title compound (8.2 mg, clear oil) was obtained from methanesulfonic acid cyclopropyl-[5-(2-fluoro-5-methyl-phenyl)-isoxazol-3-yl]-methyl ester (53 mg, 0.16 mmol), potassium carbonate (113 mg, 0.82 mmol) and piperazine-1-carboxylic acid ethyl ester (0.10 ml, 0.65 mmol) in acetonitrile (2 ml) at 80° C. overnight. Reaction mixture was cooled to room temperature, diluted with ethyl acetate (5 ml), washed with water (5 ml), dried (sodium sulfate), filtered and concentrated, in-vacuo. The crude residue was purified on silica gel using 30% ethyl acetate in hexanes. ¹H-NMR (CDCl₃), δ (ppm): 7.77 (dd, 1H), 7.21 (m, 1H), 7.08 (m, 1H), 6.75 (d, 1H), 4.16 (q, 2H), 3.49 (m, 5H), 2.70 (m, 2H), 2.48 (m, 2H), 2.40 (s, 3H), 1.27 (m, 4H), 0.80 (m, 1H), 0.51 (m, 2H), 0.21 (m, 1H).

### Example 133

### 4-{1-[5-(2-Fluoro-5-methyl-phenyl)-isoxazol-3-yl]-ethyl}-3-(R)-methyl-piperazine-1-carboxylic acid ethyl ester (2 diastereomers)

The title compounds were obtained from methanesulfonic acid 1-[5-(2-fluoro-5-methyl-phenyl)-isoxazol-3-yl]-ethyl ester (68 mg, 0.23 mmol), potassium carbonate (156 mg, 1.13 mmol) and 3-(R)-methyl-piperazine-1-carboxylic acid ethyl ester (156 mg, 0.90 mmol) in acetonitrile (3 ml) at 80° C. overnight. Reaction mixture was cooled to room temperature, diluted with dichloromethane (5 ml), sequentially washed with water (5 ml) and brine (5 ml), dried (sodium sulfate), filtered and concentrated, in-vacuo. The crude residue was purified on silica gel using 5% acetone in hexanes to separate the two diastereomers. The non-polar diastereomer, 1, was isolated as clear oil (19.6 mg). ¹H-NMR (CDCl₃), δ (ppm): 7.74 (dd, 1H), 7.21 (m, 1H), 7.09 (m, 1H), 6.72 (d, 1H), 4.32 (m, 1H), 4.12 (q, 2H), 3.79 (m, 2H), 3.03 (m, 3H), 2.40 (m, 5H), 1.38 (d, 3H), 1.27 (t, 3H), 1.16 (d, 3H). The more polar diastereomer, 2, was isolated by re-purifying the isolated impure fractions of 2, on silca gel using 15% ethyl acetate in hexanes, as clear oil (16.1 mg). ¹H-NMR (CDCl₃), δ (ppm): 7.74 (dd, 1H), 7.23 (m, 1H), 7.09 (m, 1H), 6.56 (d, 1H), 4.46 (q, 1H), 4.12 (q, 2H), 3.92 (m, 2H), 2.96 (m, 3H), 2.40 (m, 5H), 1.52 (d, 3H), 1.25 (m, 6H).

### Example 134

### 4-{1-[5-(2-Fluoro-5-methyl-phenyl)-isoxazol-3-yl]-ethyl}-3-(S)-methyl-piperazine-1-carboxylic acid ethyl ester (2 diastereomers)

The title compounds were obtained from methanesulfonic acid 1-[5-(2-fluoro-5-methyl-phenyl)-isoxazol-3-yl]-ethyl ester (68 mg, 0.23 mmol), potassium carbonate (156 mg, 1.13 mmol) and 3-(S)-methyl-piperazine-1-carboxylic acid ethyl ester (156 mg, 0.90 mmol) in acetonitrile (3 ml) at 80° C. overnight. Reaction mixture was cooled to room temperature, diluted with dichloromethane (5 ml), sequentially washed with water (5 ml) and brine (5 ml), dried (sodium sulfate), filtered and concentrated, in-vacuo. The crude residue was purified on silica gel using 5% acetone in hexanes to separate the two diastereomers. The non-polar diastereomer, 1, was isolated as clear oil (23.2 mg). ¹H-NMR (CDCl₃), δ (ppm): 7.74 (dd, 1H), 7.25 (m, 1H), 7.09 (m, 1H), 6.72 (d, 1H), 4.31 (m, 1H), 4.15 (m, 2H), 3.72 (m, 2H), 2.85 (m, 3H), 2.40 (m, 5H), 1.38 (d, 3H), 1.28 (t, 3H), 1.16 (d, 3H). The more polar diastereomer, 2, was isolated by re-purifying the isolated impure fractions of 2, on silca gel using 15% ethyl acetate in hexanes, as clear oil (19 mg). RH-NMR (CDCl₃), δ (ppm): 7.74 (dd, 1H), 7.24 (m, 1H), 7.09 (m, 1H), 6.57 (d, 1H), 4.46 (q, 1H), 4.12 (q, 2H), 3.92 (m, 2H), 2.96 (m, 3H), 2.40 (m, 5H), 1.55 (d, 3H), 1.25 (m, 6H).

### Example 135

### 4-{1-[5-(3-Chloro-phenyl)-isoxazol-3-yl]-ethyl}-3-(R)-methyl-piperazine-1-carboxylic acid ethyl ester (2 diastereomers)

The title compounds were obtained from methanesulfonic acid 1-[5-(3-chloro-phenyl)-isoxazol-3-yl]-ethyl ester (100 mg, 0.35 mmol), potassium carbonate (240 mg, 1.74 mmol) and 3-(S)-methyl-piperazine-1-carboxylic acid ethyl ester (239 mg, 1.38 mmol) in acetonitrile (3 ml) at 80° C. overnight. Reaction mixture was cooled to room temperature, diluted with dichloromethane (5 ml), sequentially washed with water (5 ml) and brine (5 ml), dried (sodium sulfate), filtered and concentrated, in-vacuo. The crude residue was purified on silica gel using 5% acetone in hexanes to separate the two diastereomers. The non-polar diastereomer, 1, was isolated as clear oil (42.6 mg). ¹H-NMR (CDCl₃), δ (ppm): 7.76 (bs, 1H), 7.68 (m, 1H), 7.41 (m, 2H), 6.61 (s, 1H), 4.28 (q, 1H), 4.16 (q, 2H), 3.68 (m, 2H), 3.03 (m, 3H), 2.35 (m, 2HH), 1.37 (d, 3H), 1.28 (t, 3H), 1.14 (d, 3H). The more polar diastereomer, 2, was isolated by re-purifying the isolated impure fractions of 2, on silca gel using 15% ethyl acetate in hexanes, as clear oil (37.5 mg). ¹H-NMR (CDCl₃), δ (ppm): 7.76 (bs, 1H), 7.66 (m, 1H), 7.41 (m, 2H), 6.44 (s, 1H), 4.43 (q, 1H), 4.10 (q, 2H), 3.76 (m, 2H), 2.97 (m, 3H), 2.29 (m, 2H), 1.50 (d, 3H), 1.25 (t, 6H).

### Example 136

### 4-{1-[5-(3-Chloro-phenyl)-isoxazol-3-yl]-ethyl}-3-(S)-methyl-piperazine-1-carboxylic acid ethyl ester (2 diastereomers)

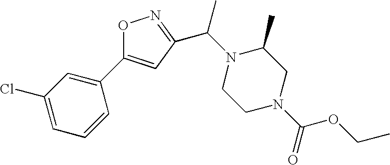

The title compounds were obtained from methanesulfonic acid 1-[5-(3-chloro-phenyl)-isoxazol-3-yl]-ethyl ester (100 mg, 0.35 mmol), potassium carbonate (240 mg, 1.74 mmol) and 3-(S)-methyl-piperazine-1-carboxylic acid ethyl ester (239 mg, 1.38 mmol) in acetonitrile (3 ml) at 80° C. overnight. Reaction mixture was cooled to room temperature, diluted with dichloromethane (5 ml), sequentially washed with water (5 ml) and brine (5 ml), dried (sodium sulfate), filtered and concentrated, in-vacuo. The crude residue was purified on silica gel using 5% acetone in hexanes to separate the two diastereomers. The isolated impure non-polar diastereomer, 1, was dissolved in dichloromethane (5 ml) and treated with hydrochloric acid (1N diethyl ether, 5 ml). The resulting mixture was concentrated in-vacuo, and the isolated residue was triturated with mixture of diethyl ether and hexanes to isolate a pale yellow oily gum. The isolated gum was treated with saturated sodium carbonate (aqueous, 5 mL), extracted with dichloromethane (3×10 ml). The combined organic phase was washed with brine (10 ml), dried (sodium sulfate), filtered and concentrated in-vacuo, to isolate, 1, as clear oil (39.7 mg). ¹H-NMR (CDCl₃), δ (ppm): 7.76 (bs, 1H), 7.68 (m, 1H), 7.41 (m, 2H), 6.61 (s, 1H), 4.28 (m, 1H), 4.16 (m, 2H), 3.70 (m, 2H), 2.93 (m, 3H), 2.38 (m, 2H), 1.38 (d, 3H), 1.28 (m, 3H), 1.15 (d, 3H). The more polar diastereomer, 2, was isolated by re-purifying the isolated impure fractions of 2, on silica gel using 50% ethyl acetate in hexanes, as clear oil (39.4 mg). ¹H-NMR (CDCl₃), δ (ppm): 7.76 (bs, 1H), 7.67 (m, 1H), 7.41 (m, 2H), 6.44 (s, 1H), 4.43 (q, 1H), 4.10 (q, 2H), 3.76 (m, 2H), 2.85 (m, 3H), 2.25 (m, 2H), 1.50 (d, 3H), 1.25 (t, 6H).

### Example 137

### 4-{1-[5-(3-Chloro-phenyl)-isoxazol-3-yl]-ethyl}-2-(R)-methyl-piperazine-1-carboxylic acid ethyl ester (2 diastereomers)

The title compounds were obtained from methanesulfonic acid 1-[5-(3-chloro-phenyl)-isoxazol-3-yl]-ethyl ester (100 mg, 0.35 mmol), potassium carbonate (240 mg, 1.74 mmol) and 2-(R)-methyl-piperazine-1-carboxylic acid ethyl ester (239 mg, 1.38 mmol) in acetonitrile (3 ml) at 80° C. overnight. Reaction mixture was cooled to room temperature, diluted with dichloromethane (5 ml), sequentially washed with water (5 ml) and brine (5 ml), dried (sodium sulfate), filtered and concentrated, in-vacuo. The crude residue was purified on silica gel using 1-5% ether in dichloromethane to separate the two diastereomers. The less polar diastereomer was pure after single column (34 mg, clear oil). ¹H-NMR (CDCl₃), δ (ppm): 7.71 (bs, 1H), 7.68 (m, 1H), 7.41 (m, 2H), 6.55 (s, 1H), 4.28 (m, 1H), 4.15 (q, 2H), 3.85 (m, 2H), 3.14 (td, 1H), 2.79 (d, 1H), 2.63 (d, 2H), 2.36 (dd, 1H), 2.24 (td, 1H), 1.44 (d, 3H), 1.26 (t, 6H). The more polar diastereomer, 2, was isolated by re-purifying the isolated impure fractions of 2, on silica gel using 1-5% ether in dichloromethane (6 mg, clear oil). ¹H-NMR (CDCl₃), δ (ppm): 7.77 (bs, 1H), 7.67 (m, 1H), 7.42 (m, 2H), 6.53 (s, 1H), 4.27 (br.s., 11H), 4.15 (q, 2H), 3.91 (br d, 1H), 3.82 (q, 1H), 3.16 (td, 1H), 2.84 (td, 1H), 2.63 (d, 1H), 2.33 (d, 1H), 2.19 (dt, 1H), 1.45 (d, 3H), 1.25 (m, 6H).

### Example 138

### 4-{1-[5-(3-Chloro-phenyl)-isoxazol-3-yl]-ethyl}-2-(S)-methyl-piperazine-1-carboxylic acid ethyl ester (2 diastereomers)

The title compounds were obtained from methanesulfonic acid 1-[5-(3-chloro-phenyl)-isoxazol-3-yl]-ethyl ester (100 mg, 0.35 mmol), potassium carbonate (240 mg, 1.74 mmol) and 2-(S)-methyl-piperazine-1-carboxylic acid ethyl ester (239 mg, 1.38 mmol) in acetonitrile (3 ml) at 80° C. overnight. Reaction mixture was cooled to room temperature, diluted with dichloromethane (5 ml), sequentially washed with water (5 ml) and brine (5 ml), dried (sodium sulfate), filtered and concentrated, in-vacuo. Flash chromatography on silica gel using 2-4% ether in dichloromethane yielded the less polar diastereomer {31 mg, clear oil; ¹H-NMR (CDCl₃), δ (ppm): 7.71 (bs, 1H), 7.68 (m, 1H), 7.41 (m, 2H), 6.55 (s, 1H), 4.28 (m, 1H), 4.15 (q, 2H), 3.85 (m, 2H), 3.14 (td, 1H), 2.79 (d, 1H), 2.63 (d, 2H), 2.36 (dd, 1H), 2.24 (td, 1H), 1.44 (d, 3H), 1.26 (t, 6H)} and the more polar diastereomer {18 mg, clear oil; ¹H-NMR (CDCl₃), δ (ppm): 7.77 (bs, 1H), 7.67 (m, 1H), 7.42 (m, 2H), 6.53 (s, 1H), 4.27 (br.s., 1H), 4.15 (q, 2H), 3.91 (br d, 1H), 3.82 (q, 1H), 3.16 (td, 1H), 2.84 (td, 1H), 2.63 (d, 1H), 2.33 (d, 1H), 2.19 (dt, 1H), 1.45 (d, 3H), 1.25 (m, 6H)}.

### Example 139

### (R)-4-[5-(3-Chloro-phenyl)-isoxazol-3-ylmethyl]-3-methyl-piperazine-1-carboxylic acid ethyl ester

(R)-4-[5-(3-Chloro-phenyl)-isoxazol-3-ylmethyl]-3-methyl-piperazine-1-carboxylic acid ethyl ester (75.5 mg, 85%, colorless oil) was obtained from methanesulfonic acid 5-(3-chloro-phenyl)-isoxazol-3-ylmethyl ester (70 mg, 0.243 mmol), potassium carbonate (134.5 mg, 0.973 mmol), and (R)-3-methyl-piperazine-1-carboxylic acid ethyl ester (125.7 mg, 0.730 mmol) in acetonitrile (4 mL) at 50° C. Purification was performed by SPE (flash) chromatography using 20-50% ethyl acetate in hexanes. ¹H NMR (CDCl₃) δ (ppm): 7.78 (m, 1H), 7.68 (m, 1H), 7.42 (m, 2H), 6.56 (s, 1H), 4.14 (q, 2H), 3.81 (m, 4H), 3.14 (m, 1H), 2.81 (m, 2H), 2.41 (m, 2H), 1.26 (t, 3H), 1.19 (d, 3H).

### Example 140

### (R)-4-[5-(2-Fluoro-5-methyl-phenyl)-isoxazol-3-ylmethyl]-3-methyl-piperazine-1-carboxylic acid ethyl ester

(R)-4-[5-(2-Fluoro-5-methyl-phenyl)-isoxazol-3-ylmethyl]-3-methyl-piperazine-1-carboxylic acid ethyl ester (80.1 mg, 90%, colorless oil) was obtained from methanesulfonic acid 5-(2-fluoro-5-methyl-phenyl)-isoxazol-3-ylmethyl ester (70 mg, 0.245 mmol), potassium carbonate (135.6 mg, 0.981 mmol), and (R)-3-methyl-piperazine-1-carboxylic acid ethyl ester (126.8 mg, 0.736 mmol) in acetonitrile (4 mL) at 50° C. Purification was performed by SPE (flash) chromatography using 10% ethyl acetate in hexanes. ¹H NMR (CDCl₃) δ (ppm): 7.75 (d, 1H), 7.24 (m, 1H), 7.08 (m, 1H), 6.68 (d, 1H), 4.13 (q, 2H), 3.83 (m, 4H), 3.13 (m, 1H), 2.86 (m, 2H), 2.40 (m, 5H), 1.26 (t, 3H), 1.19 (d, 3H).

### Example 141

### (S)-4-[5-(3-Chloro-phenyl)-isoxazol-3-ylmethyl]-3-methyl-piperazine-1-carboxylic acid ethyl ester

(S)-4-[5-(3-Chloro-phenyl)-isoxazol-3-ylmethyl]-3-methyl-piperazine-1-carboxylic acid ethyl ester (75.6 mg, 86%, colorless oil) was obtained from methanesulfonic acid 5-(3-chloro-phenyl)-isoxazol-3-ylmethyl ester (70 mg, 0.243 mmol), potassium carbonate (134.5 mg, 0.973 mmol), and (S)-3-methyl-piperazine-1-carboxylic acid ethyl ester (125.7 mg, 0.730 mmol) in acetonitrile (4 mL) at 50° C. Purification was performed by SPE (flash) chromatography using 20-50% ethyl acetate in hexanes. ¹H NMR (CDCl₃) δ (ppm): 7.78 (m, 1H), 7.68 (m, 1H), 7.42 (m, 2H), 6.56 (s, 1H), 4.14 (q, 2H), 3.81 (m, 4H), 3.14 (m, 1H), 2.81 (m, 2H), 2.41 (m, 2H), 1.26 (t, 3H), 1.19 (d, 3H).

### Example 142

### (5)-4-[5-(2-Fluoro-5-methyl-phenyl)-isoxazol-3-ylmethyl]-3-methyl-piperazine-1-carboxylic acid ethyl ester

(S)-4-[5-(2-Fluoro-5-methyl-phenyl)-isoxazol-3-ylmethyl]-3-methyl-piperazine-1-carboxylic acid ethyl ester (73.6 mg, 83%, colorless oil) was obtained from methanesulfonic acid 5-(2-fluoro-5-methyl-phenyl)-isoxazol-3-ylmethyl ester (70 mg, 0.245 mmol), potassium carbonate (135.6 mg, 0.981 mmol), and (S)-3-methyl-piperazine-1-carboxylic acid ethyl ester (126.8 mg, 0.736 mmol) in acetonitrile (4 mL) at 50° C. Purification was performed by SPE (flash) chromatography using 10% ethyl acetate in hexanes. ¹H NMR (CDCl₃) δ (ppm): 7.75 (d, 1H), 7.24 (m, 1H), 7.08 (m, 1H), 6.68 (d, 1H), 4.13 (q, 2H), 3.83 (m, 4H), 3.13 (m, 1H), 2.86 (m, 2H), 2.40 (m, 5H), 1.26 (t, 3H), 1.19 (d, 3H).

### Example 143

### 4-[5-(3-Chloro-phenyl)-oxazol-2-ylmethyl]-piperazine-1-carboxylic acid ethyl ester

4-[5-(3-Chloro-phenyl)-oxazol-2-ylmethyl]-piperazine-1-carboxylic acid ethyl ester (24 mg, 68.5%) as clear oil was obtained from 2-bromomethyl-5-(3-chloro-phenyl)-oxazole (27.3 mg, 0.1 m mol) reacted with piperazine-1-carboxylic acid ethyl ester (47.4 mg, 0.3 -mmol) and K2CO3 (41.4 mg, 0.3 mmol) in acetonitrile (1 mL) at room temperature overnight. ¹H-NMR(CDCl3) δ(ppm): 7.64 (s, 1H), 7.51 (dd, 1H), 7.29 (m, 3H), 4.13 (q, 2H), 3.79 (s, 2H), 3.54 (m, 4H), 2.58 (m, 4H) and 1.26 (t, 3H).

### Example 144

### 4-[5-(5-Chloro-2-fluoro-phenyl)-[1,2,4]oxadiazol-3-ylmethyl]-piperazine-1-carboxylic acid ethyl ester

4-[5-(5-Chloro-2-fluoro-phenyl)-[1,2,4]oxadiazol-3-ylmethyl]-piperazine-1-carboxylic acid ethyl ester (55 mg, 74%, white solid) was obtained from 5-(5-chloro-2-fluoro-phenyl)-3-chloromethyl-[1,2,4]oxadiazole (50 mg, 0.20 mmol), potassium carbonate (84 mg, 0.61 mmol), and piperazine-1-carboxylic acid ethyl ester (63 mg, 0.40 mmol) in acetonitrile (2 mL). Purification was performed by SPE (flash) chromatography using 60% ethyl acetate in hexanes. ¹H NMR (CDCl₃) δ (ppm): 8.18 (m, 1H), 7.55 (m, 1H), 7.25 (m, 1H), 4.15 (m, 2H), 3.84 (s, 2H), 3.56 (t, 4H), 2.61 (t, 4H), 1.27 (t, 3H).

### Example 145

### 4-[5-(2-Chloro-5-methyl-phenyl)-[1,2,4]oxadiazol-3-ylmethyl]-piperazine-1-carboxylic acid ethyl ester

The title compound was prepared from 3-chloromethyl-5-(2-chloro-5-methylphenyl)-[1,2,4]oxadiazole (80 mg, 0.32 mmol), potassium carbonate (136 mg, 0.96 mmol), piperazine-1-carboxylic acid ethyl ester (50 mg, 0.32 mmol) in acetonitrile (1 mL) at room temperature 72 h. Purification was performed by SPE (flash) chromatography using 30-40% ethyl acetate in hexanes afforded 52 mg (44%) of the title compound as a white solid. ¹H NMR (CDCl₃), δ (ppm): 7.90 (s, 1H), 7.44 (d, 1H), 7.32 (d, 1H), 4.14 (q, 2H), 3.83 (s, 2H), 3.55 (m, 4H), 2.61 (m, 4H), 2.40 (s, 3H), 1.25 (t, 3H).

### Example 146

### 4-{1-[5-(3-Chloro-phenyl)-[1,2,4]oxadiazol-3-yl]-ethyl}-piperazine-1-carboxylic acid ethyl ester

4-{1-[5-(3-Chloro-phenyl)-[1,2,4]oxadiazol-3-yl]-ethyl}-piperazine-1-carboxylic acid ethyl ester (113.9 mg, 60%, colorless oil) was obtained from methanesulfonic acid 1-[5-(3-chloro-phenyl)-[1,2,4]oxadiazol-3-yl]-ethyl ester (158 mg, 0.52 mmol), potassium carbonate (289 mg, 2.1 mmol), and piperazine-1-carboxylic acid ethyl ester (0.229 mL, 1.6 mmol) in acetonitrile (4 mL) at 50° C. Purification was performed by SPE (flash) chromatography first using 10% ethyl acetate in hexanes and the re-purified using 5-30% ethyl acetate in dichloromethane. ¹H NMR (CDCl₃) δ (ppm): 8.17 (s, 1H), 8.05 (d, 1H), 7.59 (m, 1H), 7.50 (m, 1H), 4.08 (m, 3H), 3.52 (t, 4H), 2.60 (t, 4H), 1.57 (d, 3H), 1.26 (t, 3H).

### Example 147

### 4-{1-[5-(3-Chloro-phenyl)-[1,2,4]oxadiazol-3-yl]-ethyl}-3-(S)-methyl-piperazine-1-carboxylic acid ethyl ester

4-{1-[5-(3-Chloro-phenyl)-[1,2,4]oxadiazol-3-yl]-ethyl}-3-(S)-methyl-piperazine-1-carboxylic acid ethyl ester (14.9 mg, 10%, light yellow oil) was obtained from methanesulfonic acid 1-[5-(3-chloro-phenyl)-[1,2,4]oxadiazol-3-yl]-ethyl ester (120 mg, 0.40 mmol), potassium carbonate (219 mg, 1.59 mmol), and (S)-3-methyl-piperazine-1-carboxylic acid ethyl ester (205 mg, 1.19 mmol) in acetonitrile (5 mL) at 50° C. Purification was performed by SPE (flash) chromatography first using 10% ethyl acetate in dichloromethane and the re-purified using 5-10% acetone in hexanes. Less-polar diastereomer ¹H NMR (CDCl₃) δ (ppm): 8.19 (m, 1H), 8.06 (m, 1H), 7.58 (m, 1H), 7.49 (m, 1H), 4.44 (q, 1H), 4.15 (q, 2H), 3.79 (m, 2H), 3.15 (m, 2H), 2.86 (m, 1H), 2.75 (m, 1H), 2.48 (m, 1H), 1.44 (d, 3H), 1.26 (t, 3H), 1.19 (d, 3H).

### Example 148

### 4-{1-[5-(3-Chloro-phenyl)-[1,2,4]oxadiazol-3-yl]-ethyl}-3-(R)-methyl-piperazine-1-carboxylic acid ethyl ester

4-{1-[5-(3-Chloro-phenyl)-[1,2,4]oxadiazol-3-yl]-ethyl}-3-(R)-methyl-piperazine-1-carboxylic acid ethyl ester (7.3 mg, 5%, light yellow oil) was obtained from methanesulfonic acid 1-[5-(3-chloro-phenyl)-[1,2,4]oxadiazol-3-yl]-ethyl ester (120 mg, 0.40 mmol), potassium carbonate (219 mg, 1.59 mmol), and (R)-3-methyl-piperazine-1-carboxylic acid ethyl ester (205 mg, 1.19 mmol) in acetonitrile (5 mL) at 50° C. Purification was performed by SPE (flash) chromatography first using 4-7% ethyl acetate in dichloromethane and the re-purified using 3-6% acetone in hexanes. Less polar diastereomer ¹H NMR (CDCl₃) δ (ppm): 8.19 (m, 1H), 8.06 (m, 1H), 7.58 (m, 1H), 7.49 (m, 1H), 4.44 (q, 1H), 4.15 (q, 2H), 3.79 (m, 2H), 3.15 (m, 2H), 2.86 (m, 1H), 2.75 (m, 1H), 2.48 (m, 1H), 1.44 (d, 3H), 1.26 (t, 3H), 1.19 (d, 3H).

### Example 149

### 4-{1-[5-(3-Chloro-phenyl)-[1,2,4]oxadiazol-3-yl]-ethyl}-3-(R)-methyl-piperazine-1-carboxylic acid ethyl ester

4-{1-[5-(3-Chloro-phenyl)-[1,2,4]oxadiazol-3-yl]-ethyl}-3-(R)-methyl-piperazine-1-carboxylic acid ethyl ester (5.9 mg, 3%, light yellow oil) was obtained from methanesulfonic acid 1-[5-(3-chloro-phenyl)-[1,2,4]oxadiazol-3-yl]-ethyl ester (150 mg, 0.495 mmol), potassium carbonate (274 mg, 1.98 mmol), and (R)-3-methyl-piperazine-1-carboxylic acid ethyl ester (205 mg, 1.19 mmol) in acetonitrile (5 mL) at 80° C. for 4 days. Purification was performed by SPE (flash) chromatography first using 5-40% ethyl acetate in dichloromethane. The more polar diastereomer was dissolved in ethyl acetate and acidified with 2N HCl (2 mL). After stirring for a few minutes, the aqueous layer was removed and the organic layer was dried over anhydrous sodium sulfate, filtered, and concentrated. The residue was re-dissolved in dichloromethane and washed with 2M sodium carbonate, dried over anhydrous sodium sulfate, filtered, and concentrated. The residue was re-purified by SPE (flash) chromatography using 4-6% acetone hexanes to afford the product. More polar diastereomer ¹H NMR (CDCl₃) δ (ppm): 8.15 (m, 1H), 8.03 (m, 1H), 7.58 (m, 1H), 7.49 (m, 1H), 4.55 (q, 1H), 4.10 (q, 2H), 3.98 (m, 2H), 3.03 (m, 2H), 2.70 (m, 1H), 2.38 (m, 1H), 2.32 (m, 1H), 1.59 (d, 3H), 1.22 (m, 6H).

### Example 150

### 4-[5-(5-Chloro-2-fluoro-phenyl)-[1,3,4]oxadiazol-2-ylmethyl]-piperazine-1-carboxylic acid ethyl ester

4-[5-(5-Chloro-2-fluoro-phenyl)-[1,3,4]oxadiazol-2-ylmethyl]-piperazine-1-carboxylic acid ethyl ester (29.2 mg, 65%, white solid) was obtained from 2-(5-chloro-2-fluoro-phenyl)-5-chloromethyl-[1,3,4]oxadiazole (30 mg, 0.121 mmol), potassium carbonate (50.3 mg, 0.364 mmol), and piperazine-1-carboxylic acid ethyl ester (0.0356 mL, 0.243 mmol) in acetonitrile (3 mL). Purification was performed by SPE (flash) chromatography using 20-60% ethyl acetate in hexanes. ¹H NMR (CDCl₃) δ (ppm): 8.08 (m, 1H), 7.52 (m, 1H), 7.24 (m, 1H), 4.15 (q, 2H), 3.97 (s, 2H), 3.56 (t, 4H), 2.63 (t, 4H), 1.27 (t, 3H).

### Example 151

### 4-{1-[5-(5-Chloro-2-fluoro-phenyl)-[1,3,4]oxadiazol-2-yl]-ethyl}-piperazine-1-carboxylic acid ethyl ester

4-{1-[5-(5-Chloro-2-fluoro-phenyl)-[1,3,4]oxadiazol-2-yl]-ethyl}-piperazine-1-carboxylic acid ethyl ester (34.2 mg, 48%, white solid) was obtained from 2-(1-bromo-ethyl)-5-(5-chloro-2-fluoro-phenyl)-[1,3,4]oxadiazole (56.8 mg, 0.186 mmol), potassium carbonate (77.1 mg, 0.558 mmol), and piperazine-1-carboxylic acid ethyl ester (0.0545 mL, 0.372 mmol) in acetonitrile (3 mL). Purification was performed by SPE (flash) chromatography using 20-50% ethyl acetate in hexanes. ¹H NMR (CDCl₃) δ (ppm): 8.06 (m, 1H), 7.52 (m, 1H), 7.23 (m, 1H), 4.18 (q, 1H), 4.10 (q, 2H), 3.56 (t, 4H), 2.54 (m, 2H), 2.46 (m, 2H), 1.62 (d, 3H), 1.25 (t, 3H).

### Example 152

### 4-[5-(2-Fluoro-5-methyl-phenyl)-[1,3,4]oxadiazol-2-ylmethyl]-piperazine-1-carboxylic acid ethyl ester

4-[5-(2-Fluoro-5-methyl-phenyl)-[1,3,4]oxadiazol-2-ylmethyl]-piperazine-1-carboxylic acid ethyl ester (29.3 mg, 38%, colorless oil) was obtained from 2-chloromethyl-5-(2-fluoro-5-methyl-phenyl)-[1,3,4]oxadiazole (50 mg, 0.221 mmol), potassium carbonate (91 mg, 0.662 mmol), and piperazine-1-carboxylic acid ethyl ester (0.032 mL, 0.221 mmol) in acetonitrile (4 mL) at 50° C. Purification was performed by SPE (flash) chromatography using 30-70% ethyl acetate in hexanes. ¹H NMR (CDCl₃) [symbol] (ppm): 7.88 (m, 1H), 7.35 (m, 1H), 7.15 (m, 1H), 4.14 (q, 2H), 3.96 (s, 2H), 3.55 (t, 4H), 2.63 (t, 4H), 2.42 (s, 3H), 1.26 (t, 3H).

### Example 153

### 4-{1-[5-(2-Fluoro-5-methyl-phenyl)-[1,3,4]oxadiazol-2-yl]-ethyl}-piperazine-1-carboxylic acid ethyl ester

4-{1-[5-(2-Fluoro-5-methyl-phenyl)-[1,3,4]oxadiazol-2-yl]-ethyl}-piperazine-1-carboxylic acid ethyl ester (19.9 mg, 52%, colorless oil) was obtained from 2-(1-Bromo-ethyl)-5-(2-fluoro-5-methyl-phenyl)-[1,3,4]oxadiazole (30 mg, 0.105 mmol), potassium carbonate (44 mg, 0.316 mmol), and piperazine-1-carboxylic acid ethyl ester (0.0154 mL, 0.105 mmol) in acetonitrile (4 mL) at 50° C. Purification was performed by SPE (flash) chromatography using 30-70% ethyl acetate in hexanes. ¹H NMR (CDCl₃) [symbol] (ppm): 7.85 (m, 1H), 7.33 (m, 1H), 7.17 (m, 1H), 4.20 (q, 1H), 4.11 (q, 2H), 3.51 (t, 4H), 2.64 (m, 2H), 2.52 (m, 2H), 2.42 (s, 3H), 1.62 (d, 3H), 1.25 (t, 3H).

### Example 154

### 4-(5-m-Tolyl-isoxazol-3-ylmethyl)-piperazine-1-carboxylic acid ethyl ester

4-(5-tributylstannanyl-isoxazol-3-ylmethyl)-piperazine-1-carboxylic acid ethyl ester (106 mg, 0.2 mmol) was mixed with Pd(PPh3)2Cl2 (0.2 mg) and 3-iodotoluene (37 mg, 0.17 mmol) in dioxane (1 mL) and the reaction mixture was heated at 110° C. overnight. The reaction mixture was directly loaded to a column and eluted with 30˜50% ethyl acetate in hexanes to give 35.2 mg (63%) of 4-(5-m-Tolyl-isoxazol-3-ylmethyl)-piperazine-1-carboxylic acid ethyl ester as yellow oil. ¹H-NMR(CDCl₃) δ (ppm): 7.59 (m, 2H), 7.36 (t, 1H), 7.25 (d, 1H), 6.56 (s, 1H), 4.14 (q, 2H), 3.66 (s, 2H), 3.52 (m, 4H), 2.51 (m, 4H), 2.42 (s, 3H) and 1.26 (t, 3H).

### Example 155

### 4-[5-(3-methoxy-phenyl)-isoxazol-3-ylmethyl]-piperazine-1-carboxylic acid ethyl ester

The title compound (29.7 mg, 50.6%, yellow sticky oil) was obtained from 4-(5-tributylstannanyl-isoxazol-3-ylmethyl)-piperazine-1-carboxylic acid ethyl ester (106 mg, 0.2 mmol) and Pd(PPh3)2Cl2 (0.2 mg) with 3-iodoanisole (39.8 mg, 0.17 mmol) in dioxane (1 mL) at 110° C. overnight. ¹H-NMR(CDCl₃) δ(ppm): 7.36 (m, 3H), 6.99 (m, 1H), 6.56 (s, 1H), 4.14 (q, 2H), 3.88 (s, 3H), 3.67 (s, 2H), 3.52 (m, 4H), 2.51 (m, 4H), 2.42 (s, 3H) and 1.27 (t, 3H).

### Example 156

### 4-[5-(3-cyano-phenyl)-isoxazol-3-ylmethyl]-piperazine-1-carboxylic acid ethyl ester

The title compound (39 mg, 67.3%, yellow solid) was obtained from 4-(5-tributylstannanyl-isoxazol-3-ylmethyl)-piperazine-1-carboxylic acid ethyl ester (106 mg, 0.2 mmol) and Pd(PPh3)2Cl2 (0.2 mg) with 3-iodobenzonitrile (38.9 mg, 0.17 mmol) in dioxane (1 mL) at 110° C. overnight. ¹H-NMR(CDCl3) δ(ppm): 8.07 (s, 2H), 8.02 (d, 1H), 7.73 (d, 1H), 7.62 (t, 1H) 6.68 (s, 1H), 4.14 (q, 2H), 3.68 (s, 2H), 3.51 (m, 4H), 2.51 (m, 4H) and 1.26 (t, 3H).

### Example 157

### 4-[5-(3-Formyl-phenyl)-isoxazol-3-ylmethyl]-piperazine-1-carboxylic acid ethyl ester

The title compound (40.5 mg, 69.5%, yellow oil) was obtained from 4-(5-tributylstannanyl-isoxazol-3-ylmethyl)-piperazine-1-carboxylic acid ethyl ester (106 mg, 0.2 mmol) and Pd(PPh3)2Cl2 (0.2 mg) with 3-iodo-benzaldehyde (38.9 mg, 0.17 mmol) in dioxane (1 mL) at 110° C. overnight. ¹H-NMR(CDCl3) δ(ppm): 10.09 (s, 1H), 8.28 (s, 1H), 8.06 (d, 1H), 7.96 (d, 1H), 7.67 (t, 1H), 6.70 (s, 1H), 4.14 (q, 2H), 3.69 (s, 2H), 3.52 (m, 4H), 2.52 (m, 4H) and 1.26 (t, 3H).

### Example 158

### 4-[5-(5-Cyano-2-fluoro-phenyl)-isoxazol-3-ylmethyl]-piperazine-1-carboxylic acid ethyl ester

The title compound (23.1 mg, 37.9%, off-white solid) was obtained from 4-(5-tributylstannanyl-isoxazol-3-ylmethyl)-piperazine-1-carboxylic acid ethyl ester (106 mg, 0.2 mmol) and Pd(PPh3)2Cl2 (0.2 mg) with 3-bromo-4-fluoro-benzonitrile (34 mg, 0.17 mmol) in dioxane (1 mL) at 110° C. overnight. ¹H-NMR(CDCl3) δ(ppm): 8.30 (dd, 1H), 7.76 (m, 1H), 7.36 (dd, 1H), 6.85 (d, 1H), 4.14 (q, 2H), 3.72 (s, 2H), 3.53 (m, 4H), 2.52 (m, 4H) and 1.27 (t, 3H).

### Example 159

### 4-[5-(5-Chloro-2-fluoro-phenyl)-isoxazol-3-ylmethyl]-piperazine-1-carboxylic acid ethyl ester

The title compound (45.4 mg, 72.7%, off-white solid) was obtained from 4-(5-tributylstannanyl-isoxazol-3-ylmethyl)-piperazine-1-carboxylic acid ethyl ester (106 mg, 0.2 mmol) and Pd(PPh3)2Cl2 (0.2 mg) with 2-bromo-4-chloro-1-fluoro-benzene (35.5 mg, 0.17 mmol) in dioxane (1 mL) at 110° C. overnight. ¹H-NMR(CDCl3) δ(ppm): 7.94 (dd, 1H), 7.40 (m, 1H), 7.16 (dd, 1H), 6.78 (d, 1H), 4.14 (q, 2H), 3.69 (s, 2H), 3.51 (m, 4H), 2.52 (m, 4H) and 1.27 (t, 3H).

### Example 160

### 4-{1-[5-(5-Chloro-2-fluoro-phenyl)-isoxazol-3-yl]-ethyl}-piperazine-1-carboxylic acid ethyl ester

The title compound (150 mg, 12.7%, off-white solid) was obtained from 4-[1-(5-tributylstannanyl-isoxazol-3-yl)-ethyl]-piperazine-1-carboxylic acid ethyl ester (1.063 g, 1.98 mmol) and Pd(PPh3)2Cl2 (19.2 mg) with 2-bromo-4-chloro-1-fluoro-benzene (368 mg, 1.76 mmol) in dioxane (10 mL) at 110° C. overnight. ¹H-NMR(CDCl3) δ(ppm): 7.94 (dd, 1H), 7.40 (m, 1H), 7.17(dd, 1H), 6.71 (d, 1H), 4.13 (q, 2H), 3.90 (q, 1H), 3.51 (m, 4H), 2.52 (m, 4H), 1.86 (d, 3H) and 1.26 (t, 3H).

### Example 170

### 4-[1-(5-m-Tolyl-isoxazol-3-yl)-ethyl]-piperazine-1-carboxylic acid ethyl ester

The title compound (31 mg, 53.1%, white solid) was obtained from 4-[1-(5-tributylstannanyl-isoxazol-3-yl)-ethyl]-piperazine-1-carboxylic acid ethyl ester (109 mg, 0.2 mmol) and Pd(PPh3)2Cl2 (2.0 mg) with 3-iodotoluene (37 mg, 0.17 mmol) in dioxane (1 mL) at 110° C. overnight. ¹H-NMR(CDCl3) δ(ppm): 7.59 (m, 2H), 7.39 (t, 1H), 7.25(dd, 1H), 6.49 (s, 1H), 4.12 (q, 2H), 3.86 (q, 1H), 3.50 (m, 4H), 2.52 (m, 4H), 2.43 (s, 3H), 1.47 (d, 3H) and 1.25 (t, 3H).

### Example 171

### 4-{1-[5-(3-Methoxy-phenyl)-isoxazol-3-yl]-ethyl}-piperazine-1-carboxylic acid ethyl ester

The title compound (26 mg, 42.6%, white solid) was obtained from 4-[1-(5-tributylstannanyl-isoxazol-3-yl)-ethyl]-piperazine-1-carboxylic acid ethyl ester (109 mg, 0.2 mmol) and Pd(PPh3)2Cl2 (2.0 mg) with 3-iodoanisole (39.8 mg, 0.17 mmol) in dioxane (1 mL) at 110° C. overnight. ¹H-NMR(CDCl3) δ(ppm): 7.37 (m, 3H); 6.99 (m, 1H), 6.50 (s, 1H), 4.12 (q, 2H), 3.88 (m, 4H), 3.48 (m, 4H), 2.52 (m, 4H), 2.43 (s, 3H), 1.47 (d, 3H) and 1.25 (t, 3H).

### Example 172

### 4-{1-[5-(3-Cyano-phenyl)-isoxazol-3-yl]-ethyl}-piperazine-1-carboxylic acid ethyl ester

The title compound (40 mg, 66.4%, white solid) was obtained from 4-[1-(5-tributylstannanyl-isoxazol-3-yl)-ethyl]-piperazine-1-carboxylic acid ethyl ester (109 mg, 0.2 mmol) and Pd(PPh3)2Cl2 (2.0 mg) with 3-iodo-benzonitrile (45.7 mg, 0.17 mmol) in dioxane (1 mL) at 110° C. overnight. ¹H-NMR(CDCl3) δ(ppm): 8.07 (s, 1H), 8.05 (d, 1H), 7.63 (d, 1H), 7.62 (t, 1H), 6.62 (s, 1H), 4.12 (q, 2H), 3.88 (q, 1H), 3.50 (m, 4H), 2.52 (m, 4H), 1.47 (d, 3H) and 1.25 (t, 3H).

### Example 173

### 4-{1-[5-(5-Cyano-2-fluoro-phenyl)-isoxazol-3-yl]-ethyl)-piperazine-1-carboxylic acid ethyl ester

The title compound (23 mg, 36.3%, white solid) was obtained from 4-[1-(5-tributylstannanyl-isoxazol-3-yl)-ethyl]-piperazine-1-carboxylic acid ethyl ester (109 mg, 0.2 mmol) and Pd(PPh3)2Cl2 (2.0 mg) with 3-bromo-4-fluoro-benzonitrile (34 mg, 0.17 mmol) in dioxane (1 mL) at 110° C. overnight. ¹H-NMR(CDCl₃) δ(ppm): 8.29 (dd, 1H), 7.74 (m, 1H), 7.35 (dd, 1H), 6.76 (d, 1H), 4.12 (q, 2H), 3.91 (m, 1H), 3.49 (m, 4H), 2.50 (m, 4H), 1.47 (d, 3H) and 1.25 (t, 3H).

### Example 174

### 4-{1-[5-(2-Methyl-pyridin-4-yl)-isoxazol-3-yl]-ethyl}-piperazine-1-carboxylic acid ethyl ester

4-{1-[5-(2-Methyl-pyridin-4-yl)-isoxazol-3-yl]-ethyl}-piperazine-1-carboxylic acid ethyl ester (30 mg, 43.5%) as white solid was obtained from 4-[1-(5-tributylstannanyl-isoxazol-3-yl)-ethyl]-piperazine-1-carboxylic acid ethyl ester (109 mg, 0.2 mmol) and Pd(PPh₃)₂Cl₂ (2.0 mg) with 4-iodo-2-methyl-pyridine (34 mg, 0.17 mmol) in dioxane (1 mL) at 110° C. overnight. ¹H-NMR(CDCl3) δ(ppm): 8.51 (d, 1H), 7.69 (s, 1H), 7.57 (dd, 1H), 6.64 (s, 1H), 4.11 (q, 2H), 3.88 (q, 1H), 3.48 (m, 4H), 2.49 (m, 4H), 2.43 (s, 3H), 1.46 (d, 3H) and 1.24 (t, 3H).

### Example 175

### 4-{1-[5-(5-Chloro-2-fluoro-phenyl)-isoxazol-3-yl]-2,2,2-trifluoro-ethyl}-piperazine-1-carboxylic acid ethyl ester

4-{1-[5-(5-Chloro-2-fluoro-phenyl)-isoxazol-3-yl]-2,2,2-trifluoro-ethyl}-piperazine-1-carboxylic acid ethyl ester (38 g, 21.8%) as pale-yellow oil was obtained from 4-(2,2,2-trifluoro-1-nitromethyl-ethyl)-piperazine-1-carboxylic acid ethyl ester (120 mg, 0.4 mmol) reacted with 4-chloro-2-ethynyl-1-fluoro-benzene (98.8 mg, 0.64 mmol), PhNCO (143.9 mg, 1.2 mmol) and triethylamine (3 drops) in benzene (3.6 mL). ¹H-NMR(CDCl3): δ (ppm): 7.96 (dd, 1H), 7.43 (m, 1H), 7.19 (dd, 1H), 6.78 (d, 1H), 4.48 (q, 1H), 4.12 (q, 2H), 3.52 (m, 4H), 2.78 (m, 2H), 2.60 (m, 2H) and 1.25 (t, 3H).

### Example 176

### 4-[5-(2-Fluoro-5-iodo-phenyl)-[1,2,4]oxadiazol-3-ylmethyl]-piperazine-1-carboxylic acid ethyl ester

N,N-Diisopropylethylamine (337 μL, 0.1.93 mmol) was added to a mixture of 2-Fluoro-5-iodobenzoyl chloride (500 mg, 1.76 mmol), 4-(N-hydroxycarbamimidoylmethyl)-piperazine-1-carboxylic acid ethyl ester (445 mg, 1.93 mmol), and dichloromethane (5 mL) and the resulting mixture was stirred at room temperature overnight. The reaction mixture was diluted with ethyl acetate, washed with water and brine, dried over anhydrous sodium sulfate, filtered and concentrated. To the resulting crude residue was added, THF (1 mL) and tetrabutylammoniumfluoride (2 mL, 1.93 mmol of a 1 M solution in THF) and the mixture was stirred for 72 hours at room temperature to complete the cyclization of the oxadiazole. The title compound was obtained by SPE (flash) chromatography using 50% ethyl acetate in hexanes to give 133 mg (17% yield over two steps) of the title compound as a white solid. ¹H NMR (CDCl₃), δ (ppm): 8.47 (d, 1H), 7.85 (m, 1H), 7.06 (t, 1H), 4.13 (q, 2H), 3.82 (s, 2H), 3.55 (m, 4H), 2.60 (m, 4H), 1.20 (t, 3H).

Examples below were prepared as described for example 176.

### Example 177

### 4-[5-(2-Hydroxy-5-methyl-phenyl)-[1,2,4]oxadiazol-3-ylmethyl]-piperazine-1-carboxylic acid ethyl ester

N,N-Diisopropylethylamine (454 μL, 2.6 mmol) was added to a mixture of 2-Hydroxy-5-methyl-benzoyl chloride (221 mg, 1.3 mmol), 4-(N-hydroxycarbamimidoylmethyl)-piperazine-1-carboxylic acid ethyl ester (300 mg, 1.3 mmol), and dichloromethane (2 mL) and the resulting mixture was stirred at room temperature overnight. The reaction mixture was diluted with ethyl acetate, washed with water and brine, dried over anhydrous sodium sulfate, filtered and concentrated. To the resulting crude residue was added, THF (1 mL) and tetrabutylammoniumfluoride (1.43 mL, 1.43 mmol of a 1 M solution in THF) and the mixture was stirred for 72 hours at room temperature to complete the cyclization of the oxadiazole. The title compound was obtained by SPE (flash) chromatography using 505 ethyl acetate in hexanes to give 72 mg (16% yield over two steps) of the title compound as a white solid. ¹H NMR (CDCl₃), δ (ppm): 10.1 (s, 1H), 7.71 (s, 1H), 7.30 (d, 1H), 7.00 (d, 1H), 4.15 (q, 2H), 3.84 (s, 2H), 3.54 (m, 4H), 2.60 (m, 4H), 1.25 (t, 3H).

### Example 178

### 4-[5-(5-Chloro-2-hydroxy-phenyl)-[1,2,4]oxadiazol-3-ylmethyl]-piperazine-1-carboxylic acid ethyl ester

N,N-Diisopropylethylamine (232 μL, 1.33 mmol) was added to a mixture of 5-Chloro-2-hydroxy-benzoyl chloride (190 mg, 1.21 mmol), 4-(N-hydroxycarbamimidoylmethyl)-piperazine-1-carboxylic acid ethyl ester (307 mg, 1.33 mmol), and dichloromethane (5 mL) and the resulting mixture was stirred at room temperature overnight. The reaction mixture was diluted with ethyl acetate, washed with water and brine, dried over anhydrous sodium sulfate, filtered and concentrated. To the resulting crude residue was added, THF (1 mL) and tetrabutylammoniumfluoride (1.33 mL, 1.33 mmol of a 1 M solution in THF) and the mixture was stirred for 72 hours at room temperature to complete the cyclization of the oxadiazole. The title compound was obtained by SPE (flash) chromatography using 50% ethyl acetate in hexanes to give 58 mg (13% yield over two steps) of the title compound as a white solid. ¹H NMR (CDCl₃), δ (ppm): 10.22 (s, 1H), 7.80 (s, 1H), 7.37 (dt, 1H), 6.99 (d, 1H) 4.07 (q, 2H), 3.75 (s, 2H), 3.46 (m, 4H), 2.52 (m, 4H), 1.18 (t, 3H).

### Example 179 (AR-P132570)

### 1-[5-(3-Chloro-phenyl)-[1,2,4]oxadiazol-3-ylmethyl]-piperazine

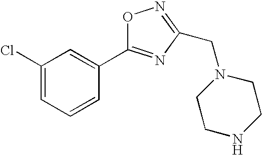

To a solution of 3-chloromethyl-5-(3-chloro-phenyl)-[1,2,4]oxadiazole (114 mg, 0.50 mmol) in DMF (2 mL) was piperazine (215 mg, 2.50 mmol) and potassium carbonate (104 mg, 0.75 mmol) added. The reaction mixture was stirred over night, diluted with ethyl acetate and washed with water followed by aqueous saturated sodium chloride. The organic phase was dried over MgSO₄ and evaporated. The title compound (66 mg, 48%) was isolated by flash chromatography using 3-20% methanol in chloroform. ¹H NMR (CDCl₃) δ (ppm): 8.16 (m, 1H), 8.04 (m, 1H), 7.56 (m, 1H), 7.47 (t, 1H), 3.77 (s, 2H), 2.98 (m, 4H), 2.64 (m, 4H).

### Example 180

### 4-(N-Hydroxycarbamimidoyl)-piperazine-1-carboxylic acid ethyl ester

Cyanogen bromide (0.80 g, 7.51 mmol) was dissolved in anhydrous diethyl ether (25 mL) and ethyl I-piperazinecarboxylate (1.00 ml, 6.83 mmol) was added. The resulting mixture was stirred over night under an atmosphere of argon and then washed with aqueous saturated sodium bicarbonate followed by aqueous saturated sodium chloride. The organic phase was dried over MgSO₄ and evaporated. The residue was dissolved in dioxane (20 mL), pyridine (1.53 ml, 18.89 mmol) and hydroxylamine hydrochloride (0.39 g, 5.67 mmol) was added. The reaction mixture was stirred for 3 days at ambient temperature and then evaporated. The title compound (0.48 g, 2.21 mmol) was obtained by flash chromatography using 5-10% methanol in chloroform. 1H NMR (CDCl₃) δ (ppm): 4.14 (q, 2H), 3.59 (m, 4H), 3.49 (m, 4H), 1.26 (t, 3H).

### Example 181

### Ethyl 4-(-amino{[(3-chlorobenzoyl)oxy]imino}methyl)piperazine-1-carboxylate

4-(N-Hydroxycarbamimidoyl)-piperazine-1-carboxylic acid ethyl ester (43 mg, 0.20 mmol) and 3-chlorobenzoic acid (38 mg, 0.24 mmol) were dissolved in DMF (1 mL). DIPEA (70 μL, 0.40 mmol) followed by HBTU (91 mg, 0.24 mmol) was added and the reaction mixture was stirred for 2 h. The reaction mixture was diluted with ethyl acetate and washed with water followed by aqueous saturated sodium chloride, the organic phase was dried over MgSO₄ and evaporated. The title compound (12 mg, 17%) was obtained by flash chromatography using 2% methanol in chloroform. ¹H NMR (CDCl₃) δ (ppm): 7.92 (m, 1H), 7.84 (m, 1H), 7.47 (m, 1H), 7.33 (t, 1H), 4.52 (s, 2H), 4.09 (q, 2H), 3.48 (m, 4H), 3.25 (m, 4H), 1.21 (t, 3H).

### Example 182

### 5-Chloromethyl-3-(2,5-difluoro-phenyl)-[1,2,4]oxadiazole

N′-[(Chloroacetyl)oxy]-2,5-difluorobenzenecarboximidamide was dissolved in anhydrous DMF (50 mL) and heated to 120° C. for 5 h. After cooling to room temperature, the reaction mixture was diluted with ethyl acetate and washed with water followed by brine. The organic phase was dried over MgSO₄ and evaporated. The title compound (1.19 g, 76%) was isolated by flash chromatography using 25% ethyl acetate in heptane. ¹H NMR (CDCl₃) δ (ppm): 7.70 (m, 1H), 7.18 (m, 2H), 4.78 (s, 2H).

Example 183 prepared as in example 14.

### Example 183

### {1-[5-(2-Fluoro-5-methyl-phenyl)-[1,2,4]oxadiazol-3-ylmethyl]-piperidin-2-yl}-methylamine hydrochloride

The title compound was obtained in 53% yield starting from 3-chloromethyl-5-(2-fluoro-5-methyl-phenyl)-[1,2,4]oxadiazole and (0.44 mmol) 0.57 mmol piperidin-2-ylmethyl-carbamic acid t-butyl ester (0.57 mmol) in DMF using DIPEA as base. The resulting residue was stirred in 5 mL 1 M HCl in diethyl ether over night in order to remove the Boc protecting group. MS (ESI) m/z: 304.9 (M+1)

### Example 184

### 4-[5-(3-Chloro-phenyl)-[1,2,4]oxadiazol-3-ylmethyl]-piperazine-1-carbothioic acid S-ethyl ester

To 1-[5-(3-chloro-phenyl)-[1,2,4]oxadiazol-3-ylmethyl]-piperazine (20 mg, 72 μmol) and potassium carbonate (20 mg, 144 μmol) in anhydrous THF (1 mL) was ethyl chlorothiolformate (15 μL, 144 μmol) added. The mixture was stirred over night under an argon atmosphere. Ethyl acetate was added and the resulting mixture was washed consecutively with water and aqueous saturated sodium chloride. The organic phase was dried over MgSO₄ and evaporated. The title compound (19 mg, 70%) was isolated by flash chromatography using 20% ethyl acetate in heptane. 1H NMR (CDCl₃) δ (ppm): 8.10 (t, 1H), 7.97 (m, 1H), 7.51 (m, 1H), 7.41 (t, 1H), 3.73 (s, 2H), 3.55 (m, 4H), 2.84 (q, 2H), 2.56 (t, 4H), 1.21 (t, 3H).

### Example 185

### 1-{1-[5-(3-Chloro-phenyl)-[1,2,4]oxadiazol-3-ylmethyl]-piperidin-4-yl}-1,4-dihydro-benzo[d][1,3]oxazin-2-one

1-Piperidin-4-yl-1,4-dihydro-benzo[d][1,3]oxazin-2-one hydrochloride (described in Bell, I. M. et al J. Med. Chem. (1998) 2146-2163) (30 mg, 0.11 mmol) and 3-chloromethyl-5-(3-chloro-phenyl)-[1,2,4]oxadiazole (23 mg, 0.10 mmol) was dissolved in anhydrous DMF (1 mL). DIPEA (26 μL, 0.15 mmol) and potassium carbonate (28 mg, 0.20 mmol) was added, the mixture was stirred at ambient temperature for 20 h. The reaction mixture was diluted with ethyl acetate and washed consecutively with water and aqueous saturated sodium chloride. The organic phase was dried over MgSO₄ and evaporated. The title compound was isolated (33 mg, 78%) by flash chromatography using 2% methanol in chloroform.

¹H NMR (CDCl₃) δ ppm: 8.11 (m, 1H), 7.98 (m, 1H), 7.50 (m, 1H), 7.41 (t, 1H), 7.24 (m, 1H), 7.09 (m, 2H), 6.99 (t, 1H), 4.99 (s, 2H), 3.96 (m, 1H), 3.80 (s, 2H), 3.12 (m, 2H), 2.73 (qd, 2H), 2.36 (t, 2H), 1.78 (d, 2H).

Example 186 prepared as described for Example 185.

### Example 186

### 1-{1-[5-(2-Fluoro-5-methyl-phenyl)-[1,2,4]oxadiazol-3-ylmethyl]-piperidin-4-yl}-1,4-dihydro-benzo[d][1,3]oxazin-2-one

The title compound was prepared as 1-{1-[5-(3-chloro-phenyl)-[1,2,4]oxadiazol-3-ylmethyl]-piperidin-4-yl}-1,4-dihydro-benzo[d][1,3]oxazin-2-one from 1-piperidin-4-yl-1,4-dihydro-benzo[d][1,3]oxazin-2-one hydrochloride (59 mg, 0.22 mmol), 3-chloromethyl-5-(2-fluoro-5-methyl-phenyl)-[1,2,4]oxadiazole (45 mg, 0.20 mmol), DIPEA (52 μL, 0.30 mmol) and potassium carbonate (55 mg, 0.40 mmol). The title compound (67 mg, 79%) was obtained by flash chromatography using 2% methanol in chloroform.

1H NMR (CDCl₃) δ (ppm): 7.95 (m, 1H), 7.49 (m, 1H), 7.36 (m, 1H); 7.24 (m, 3H), 7.10 (t, 1H), 5.11 (s, 2H), 3.99 (tt, 1H), 3.85 (s, 2H), 3.18 (m, 2H), 2.77 (qd, 2H), 2.47 (m, 2H), 2.41 (s, 3H), 1.85 (m, 2H).

### Example 187

### 4-[5-(3-Chloro-phenyl)-[1,2,4]oxadiazol-3-yl]-piperazine-1-carboxylic acid ethyl ester

Ethyl 4-(-amino{[(3-chlorobenzoyl)oxy]imino} methyl)piperazine-1-carboxylate (12 mg, 34 μmol) was dissolved in anhydrous THF (1 mL) and TBAF (1M in THF, 34 μL, 34 μmol) was added. The reaction mixture was stirred over night and then concentrated. The title compound was obtained by flash chromatography using 25% ethyl acetate in heptane. 1H NMR (CDCl₃) δ (ppm): 8.00 (m, 1H), 7.88 (m, 1H), 7.47 (m, 1H), 7.38 (t, 1H), 4.11 (q, 2H), 3.54 (m, 4H), 3.46 (m, 4H), 1.22 (t, 3H).

Example 188 prepared by the method described in example 14.

### Example 188

### {1-[5-(2-Fluoro-5-methyl-phenyl)-[1,2,4]oxadiazol-3-ylmethyl]-piperidin-2-yl}-acetic acid ethyl ester

The title compound (30 mg, 83%) was obtained from 3-chloromethyl-5-(2-fluoro-5-methyl-phenyl)-[1,2,4]oxadiazole (24 mg) and piperidin-2-yl-acetic acid ethyl ester hydrochloride (described in Clemo et. al, J. Chem. Soc. 1935, 1743) (21 mg). ¹HNMR (CDCl₃), δ (ppm): 7.94 (d, 1H), 7.38 (m, 1H), 7.14 (t, 1H), 4.15 (q, 2H), 3.97 (q, 2H), 3.02-2.81 (m, 3H), 2.60 (m, 2H), 2.40 (s, 3H), 1.83-1.30 (m, 6H), 1.24 (t, 3H).

### Example 189

### (1-[5-(2-Fluoro-5-methyl-phenyl)-[1,2,4]oxadiazol-3-ylmethyl]-piperidin-2-ylmethyl}-carbamic acid ethyl ester

To {1-[5-(2-fluoro-5-methyl-phenyl)-[1,2,4]oxadiazol-3-ylmethyl]-piperidin-2-yl}-methylamine hydrochloride (0.18 mmol) and 0.44 mmol DIPEA (0.44 mmol) in dichloromethane (3 mL) was added ethyl chloroformate (0.23 mmol) and the mixture was stirred over night at room temperature. The title compound was obtained by SPE on silica gel using 30% ethyl acetate in heptane in 85% yield. ¹H NMR (CDCl₃) δ (ppm): 7.93 (d, 1H), 7.35 (m, 1H), 7.12 (m, 1H), 5.67 (s, 1H), 4.11 (q, 2H), 3.97 (d, 1H), 3.87 (d, 1H), 3.55 (m, 1H), 3.40-3.31 (m, 1H), 2.95 (m, 1H), 2.57 (m, 1H), 2.48-2.37 (m overlapping with s, 4H)), 1.75-1.45 (m, 5H), 1.35-1.19 (m overlapping with t, 4H).

Pharmaceutical Examples

Assay of Group I receptor antagonist activity

For FLIPR analysis, cells were seeded on collagen coated clear bottom 96-well plates with black sides and analysis of [Ca²⁺]ᵢ mobilization was performed 24 hours following seeding. Cell cultures in the 96-well plates were loaded with a 4 μM solution of acetoxymethyl ester form of the fluorescent calcium indicator fluor-3 (Molecular Probes, Eugene, Oreg.) in 0.01% pluronic. All assays were performed in a buffer containing 127 mM NaCl, 5 mM KCl, 2 mM MgCl₂, 0.7 mM NaH₂PO₄, 2 mM CaCl₂, 0.422 mg/ml NaHCO₃, 2.4 mg/ml HEPES, 1.8 mg/ml glucose and 1 mg/ml BSA Fraction IV (pH 7.4). FLIPR experiments were done using a laser setting of 0.800 W and a 0.4 second CCD camera shutter speed with excitation and emission wavelengths of 488 nm and 562 nm, respectively. Each FLIPR experiment was initiated with 160 μL of buffer present in each well of the cell plate. A 40 μL addition from the antagonist plate was followed by a 50 μL addition from the agonist plate. After each addition the fluorescence signal was sampled 50 times at 1 second intervals followed by 3 samples at 5 second intervals. Responses were measured as the peak height of the response within the sample period. EC₅₀/IC₅₀ determinations were made from data obtained from 8 point concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

Measurement of Inositol Phosphate Turnover in Intact Whole Cells

GHEK stably expressing the human mGluR5d receptor were seeded onto 24 well poly-L-lysine coated plates at 40×10⁴ cells/well in media containing 1 μCi/well [3H]myo-inositol. Cells were incubated overnight (16 h), then washed three times and incubated for 1 hour at 37° C. in HEPES buffered saline (146 mM NaCl, 4.2 mM KCl, 0.5 mM MgCl₂, 0.1% glucose, 20 mM HEPES, pH 7.4) supplemented with 1 unit/ml glutamate pyruvate transaminase and 2 mM pyruvate. Cells were washed once in HEPES buffered saline and pre-incubated for 10 minutes in HEPES buffered saline containing 10 mM LiCl. Compounds (agonists) were added and incubated at 37° C. for 30 minutes. Antagonist activity was determined by pre-incubating test compounds for 15 minutes, then incubating in the presence of glutamate (80 μM) or DHPG (30 μM) for 30 minutes. The reaction was terminated by the addition of 0.5 mL perchloric acid (5%) on ice, with incubation at 4° C. for at least 30 minutes. Samples were collected in 15 mL Falcon tubes and inositol phosphates were separated using Dowex columns, as described below.

Assay For Inositol Phosphates Using Gravity-Fed Ion-Exchange Columns

a) Preparation of Ion-Exchange Columns

Ion-exchange resin (Dowex AG1-X8 formate form, 200-400 mesh, BIORAD) was washed three times with distilled water and stored at 4° C. 1.6 mL resin was added to each column and washed with 3 mL 2.5 mM HEPES, 0.5 mM EDTA, pH 7.4.

b) Sample Treatment

Samples were collected in 15 mL Falcon tubes and neutralized with 0.375 M HEPES, 0.75 M KOH. 4 mL of HEPES/EDTA (2.5/0.5 mM, pH 7.4) were added to precipitate the potassium perchlorate. Supernatant was added to the prepared Dowex columns.

c) Inositol Phosphate Separation

Elute glycero phosphatidyl inositols with 8 mL 30 mM ammonium formate. Elute total inositol phosphates with 8 mL 700 mM ammonium formate/100 mM formic acid and collect eluate in scintillation vials. Count eluate mixed with 8 mL scintillant.

### Results

Typical IC₅₀ values as measured in the assays described above are 10 μM or less. In one aspect of the invention the IC₅₀ is below 2 μM. In another aspect of the invention the IC₅₀ is below 0.2 μM. In a further aspect of the invention the IC₅₀ is below 0.05 μM.

1. A compound having the formula I 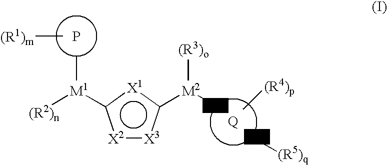
wherein:
P is selected from phenyl, pyridyl and thiophenyl.;
R¹ is selected from the group consisting of Cl, F, Me, Meo, OH, CN, furyl, OCF₃,CHO, SMe and CF₃;
M¹ is a bond;
X¹, X² and X³ are independently selected from the group consisting of CR, CO, N, NR, O and S;
R is selected from the group consisting of hydrogen, C₀₋₃alkyl, halo, C₀₋₃alkylOR⁵, C₀₋₃alkylNR⁵R⁶, C₀₋₃alkyl(CO)OR⁵, C₀₋₃alkylNR⁵R⁶ and C₀₋₃alkylaryl;
R² is selected from the group consisting of hydrogen, hydroxy, oxo, ═NR⁶, ═NOR⁶, C₁₋₄alkylhalo, halo, C₁₋₄alkyl, OC₁₋₄alkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₁₋₄alkyl, C₀₋₄alkylcyano, C₁₋₄alkylOR⁶ and C₀₋₄alkylNR⁶R⁷;
M² is selected from the group consisting of a bond, C₁alkyl, and CO;
R³ is selected from the group consisting of hydrogen, hydroxy, oxo, ═NR⁶, ═NOR⁶, C₁₋₄alkylhalo, halo, C₁₋₄alkyl, OC₁₋₄alkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₀₋₄alkylcyano, C₁₋₄alkylOR⁶ and C₀₋₄akylNR⁶R⁷;
X⁴ is N;
X⁵ is N;
Q is a 6-membered ring or bicycle containing two N atoms, wherein said ring or bicycle may be fused with a 5- or 6-membered ring containing one or more atoms independently selected from C, N, O or S and wherein the fused ring may be substituted by one or more A;
R⁴ is selected from the group consisting of hydrogen, hydroxy, halo, nitro, oxo, C₁₋₆alkylhalo, C₁₋₆alkyl, OC₁₋₆alkyl, C₀₋₆alkylC₃₋₆cycloalkyl, C₀₋₆alkylaryl, OC₀₋₆alkylaryl, (CO)R⁶, O(CO)R⁶, C₁₋₆alkylOR⁶, OC₂₋₆alkylOR⁶, C₁₋₆alkyl(CO)R⁶, OC₁₋₆alkyl(CO)R⁶, C₀₋₆alkylCO₂R⁶, OC₁₋₆alkylCO₂R⁶, C₀₋₆alkylcyano, OC₁₋₆alkylcyano, C₀₋₆alkylNR⁶R⁷, OC₂₋₆alkylNR⁶R⁷, C₀₋₆alkyl(CO)NR⁶R⁷, OC₀₋₆alkyl(CO)NR⁶R⁷, C₀₋₆alkylNR⁶(CO)R⁷, OC₂₋₆alkylNR⁶(CO)R⁷, C₀₋₆alkylNR⁶(CO)NR⁶R⁷, C₀₋₆alkylSR⁶, OC₂₋₆alkylSR⁶, C₀₋₆alkyl(SO)R⁶, OC₂₋₆alkyl(SO)R⁶, C₀₋₆alkylSO₂R⁶, OC₀₋₆alkylSO₂R⁶, C₀₋₆alkyl(SO₂)NR⁶R⁷, OC₀₋₆alkyl(SO₂)NR⁶R⁷, C₀₋₆alkylNR⁶(SO₂)R⁷, OC₂₋₆alkylNR⁶(SO₂)R⁷, NR⁶OR⁷, NR⁶(CO)OR⁷, SO₃R⁶ and a 5- or 6-membered ring containing one or more atoms independently selected from C, N, O or S, wherein said ring may be substituted by one or more A;
R⁵ is selected from the group consisting of (CO)OR⁶ and (CS)OR⁶, (CO)SR⁶, CONR6R7 wherein, R⁶ are independently selected from the group consisting of methyl and ethyl, propyl, ipropyl, n-butyl and i-butyl;
R⁶ and R⁷ are independently selected from hydrogen, C₁₋₆alkyl, C₀₋₆alkylC₃₋₆cycloalkyl, C₀₋₆alkylaryl, C₁₋₆alkylheteroaryl and a 5- or 6-membered ring containing one or more atoms independently selected from C, N, O or S, and wherein R⁶ and R⁷ may together form a 5- or 6-membered ring containing one or more atoms independently selected from C, N, O or S;
wherein any C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl defined under R¹, R², R³, R⁴, R⁵, R⁶ and R⁷ may be substituted by one or more A;
A is selected from the group consisting of hydrogen, hydroxy, oxo, halo, nitro, C₁₋₆alkylhalo, OC₁₋₆alkylhalo, C₁₋₆alkyl, C₀₋₄alkylC₃₋₆cycloalkyl, C₂₋₆alkenyl, OC₁₋₆alkyl, C₀₋₃alkylaryl, C₁₋₆alkylOR⁶, OC₂₋₆alkylOR⁶, C₁₋₆alkylSR⁶, OC₂₋₆alkylSR⁶, (CO)R⁶, O(CO)R⁶, OC₂₋₆alkylcyano, C₀₋₆alkylcyano, C₀₋₆alkylCO₂R⁶, OC₁₋₆alkylCO₂R⁶, O(CO)OR⁶, OC₁₋₆alkyl(CO)R⁶, C₁₋₆alkyl(CO)R⁶, NR⁶OR⁷, C₀₋₆alkylNR⁶R⁷, OC₂₋₆alkylNR⁶R⁷, C₀₋₆alkyl(CO)NR⁶R⁷, OC₁₋₆alkyl(CO)NR⁶R⁷, OC₂₋₆alkylNR⁶(CO)R⁷, C₀₋₆alkylNR⁶(CO)R⁷, C₀₋₆alkylNR⁶(CO)NR⁶R⁷, O(CO)NR⁶R⁷, NR⁶(CO)OR⁷, C₀₋₆alkyl(SO₂)NR⁶R⁷, OC₂₋₆alkyl(SO₂)NR⁶R⁷, C₀₋₆alkylNR⁶(SO₂)R⁷, OC₂₋₆alkylNR⁶(SO₂)R⁷, SO₃R⁶, C₁₋₆alkylNR⁶(SO₂)NR⁶R⁷, OC₂₋₆alkyl(SO₂)R⁶, C₀₋₆alkyl(SO₂)R⁶, C₀₋₆alkyl(SO)R⁶ and OC₂₋₆alkyl(SO)R⁶;
m is selected from 1 and 2;
n is 0;
o is selected from 0, and 1;
p is selected from 0, 1 and 2; and
q is selected from 0 and 1;
or salt thereof
with the proviso that the compound is not:
1-Piperazinecarboxylic acid, 4-[5-(4-chlorophenyl)-4-(4-pyridinyl)-1H-pyrazol-3-yl]-methyl ester,
1-Piperazinecarboxylic acid, 4-[5-phenyl-4-(4-pyridinyl)-1H-pyrazol-3-yl]-ethyl ester,
1-Piperazinecarboxylic acid-4-[[4-(10Hphenothiazine-2-yl)-2-thiazolyl]methyl]-methyl ester,
1-piperazinecarboxylic acid, 4-[[4-[3,5-bis(1,1-dimethylethyl)-4-hydroxyphenyl]-2-thizolyl]methyl]-methyl ester monohydrochloride,
1-piperazinecarboxylic acid, 4-[[4-[3,5-bis(1,1-dimethylethyl)-4-hydroxyphenyl]-2-thizolyl]methyl]-methyl ester,
1-Piperazinecarboxylic acid, 4-[[5-[4-(trifluoromethyl)-3-pyridinyl]-1,2,4-oxadiazol-3-yl]carbonyl]-ethyl ester,
1-Piperazinecarboxylic acid, 4-[1-(acetylamino)-4-(4-bromophenyl)-1H-imidazol-2-yl]-ethyl ester,
1-Piperazinecarboxylic acid, 4-[[2-(3-pyridinyl)-4-thiazolidinyl]carbonyl]-ethyl ester,
1-Piperazinecarboxylic acid, 4-[[2-(3-pyridinyl)-4-thiazolidinyl]carbonyl]-ethyl ester dihydrochloride,
1-Piperazinecarboxylic acid, 4-[5-(1-methyl-5-nitro-1H-imidazol-2-yl)-1,3,4-thiadiazol-2-yl]-ethyl ester, and
1-Piperazinecarboxylic acid, 4(4,5-diphenyl-2-oxazolyl)-ethyl ester.

2. A compound according to claim 1 wherein q is 0.

3. A compound according to claim 1 or 2 wherein X³ is N.

4. A compound according to any one of claims 1 to 3 wherein X² is 0.

5. A compound according to any one of claims 1 to 4 wherein X¹ is selected from N and C.

6. A compound according to any one of claims 1 to 5 wherein m is 1.

7. A compound according to any one of claims 1 to 6 wherein R is selected from the group consisting of Cl, F, Me, MeO, OH and CN.

8. A compound selected from the group consisting of
4-(5-m-Tolyl-[1,2,4]oxadiazol-3-ylmethyl)-piperazine-1-carboxylic acid ethyl ester hydrochloride,
4-[5-(3-Methoxyphenyl)-[1,2,4]oxadiazol-3-ylmethyl)-piperazine-1-carboxylic acid ethyl ester hydrochloride,
4-[5-(3-Trifluoromethyl-phenyl)-[1,2,4]oxadiazol-3-ylmethyl]-piperazine-1-carboxylic acid ethyl ester,
4-[5-(3-Cyano-phenyl)-[1,2,4]oxadiazol-3-ylmethyl]-piperazine-1-carboxylic acid ethyl ester),
4-[5-(3-Fluoro-phenyl)-[1,2,4]oxadiazol-3-ylmethyl]-piperazine-1-carboxylic acid ethyl ester,
4-[5-(3-Iodo-phenyl)-[1,2,4]oxadiazol-3-ylmethyl]-piperazine-1-carboxylic acid ethyl ester,
4-[5-(3-Chloro-phenyl)-[1,2,4]oxadiazol-3-ylmethyl]-piperazine-1-carboxylic acid ethyl ester,
4-[5-(3-Trifluoromethoxy-phenyl)-[1,2,4]oxadiazol-3-ylmethyl]-piperazine-1-carboxylic acid ethyl ester,
4-[5-(3-Bromo-phenyl)-[1,2,4]oxadiazol-3-ylmethyl]-piperazine-1-carboxylic acid ethyl ester,
4-(5-m-Tolyl-[1,2,4]oxadiazol-3-ylmethyl)-piperazine-1-carboxylic acid methyl ester,
4-(5-m-Tolyl-[1,2,4]oxadiazol-3-ylmethyl)-piperazine-1-carboxylic acid propyl ester,
4-(5-m-Tolyl-[1,2,4]oxadiazol-3-ylmethyl)-piperazine-1-carboxylic acid butyl ester,
4-[5-(3-Methoxy-phenyl)-[1,2,4]oxadiazol-3-ylmethyl]-2-methyl-piperazine-1-carboxylic acid ethyl ester,
4-(5-m-Tolyl-[1,2,4]oxadiazol-3-ylmethyl)-piperazine-1-carboxylic acid isopropyl ester,
4-[1-(5-(3-Methyl-phenyl)-[1,2,4]oxadiazol-3-yl)-ethyl]-piperazine-carboxylic acid ethyl ester or
4-[5-(3-Furan-3-yl-phenyl)-[1,2,4]oxadiazol-3-ylmethyl]-piperazine-1-carboxylic acid ethyl ester,
4-{Cyano-[5-(2-fluoro-5-methyl-phenyl)-isoxazol-3-yl]-methyl}-piperazine-1-carboxylic acid ethyl ester,
4-[5-(3-Chloro-phenyl)-[1,2,4]oxadiazol-3-ylmethyl]-2-oxo-piperazine-1-carboxylic acid ethyl ester,
4-[1-(5-m-Tolyl-[1,2,4]oxadiazol-3-yl)-ethyl]-piperazine-1-carboxylic acid ethyl-methyl-amide,
(R)-and (S)-4-[1-(5-(3-Methyl-phenyl)-[1,2,4]oxadiazol-3-yl)-ethyl]-piperazine-carboxylic acid ethyl ester,
(R)-and (S)-4-[1-(5-(3-Methyl-phenyl)-[1,2,4]oxadiazol-3-yl)-ethyl]-piperazine-carboxylic acid ethyl ester,
4-{1-[5-(3-Chloro-phenyl)-[1,2,4]oxadiazol-3-yl]-propyl}-piperazine-1-carboxylic acid ethyl ester,
(S)-4-{1-[5-(5-Chloro-2-fluoro-phenyl)-[1,2,4]oxadiazol-3-yl]-ethyl}-piperazine-1-carboxylic acid ethyl ester,
(S)-{1-[5-(2-Fluoro-5-methyl-phenyl)-[1,2,4]oxadiazol-3-yl]-ethyl}-piperazine-1-carboxylic acid ethyl ester,
(S)-4-{1-[5-(3-Chloro-phenyl)-[1,2,4]oxadiazol-3-yl]-ethyl}-piperazine-1-carboxylic acid ethyl ester,
(R)-4-[5-(2-Fluoro-5-methyl-phenyl)-[1,2,4]oxadiazol-3-ylmethyl]-2-methyl-piperazine-1-carboxylic acid ethyl ester,
(S)-4-[5-(2-Fluoro-5-methyl-phenyl)-[1,2,4]oxadiazol-3-ylmethyl]-2-methyl-piperazine-1-carboxylic acid ethyl ester,
(R)-3-Methyl-4-(5-m-tolyl-[1,2,4]oxadiazol-3-ylmethyl)-piperazine-1-carboxylic acid ethyl ester,
(S)-3-Methyl-4-(5-m-tolyl-[1,2,4]oxadiazol-3-ylmethyl)-piperazine-1-carboxylic acid ethyl ester,
4-[5-(3-Methylsulfanyl-phenyl)-[1,2,4]oxadiazol-3-ylmethyl]-piperazine-1-carboxylic acid ethyl ester,
4-[5-(2-Fluoro-5-methyl-phenyl)-[1,2,4]oxadiazol-3-ylmethyl]-piperazine-1-carboxylic acid ethyl ester,
4-[5-(3-Chloro-phenyl)-isoxazol-3-ylmethyl]-piperazine-1-carboxylic acid ethyl ester,
4-[5-(2-Fluoro-5-methyl-phenyl)-[1,2,4]oxadiazol-3-yl-(R)-methyl]-3-methyl-piperazine-1-carboxylic acid ethyl ester,
4-[5-(2-Fluoro-5-methyl-phenyl)-[1,2,4]oxadiazol-3-yl-(S)-methyl]-3-methyl-piperazine-1-carboxylic acid ethyl ester,
4-[5-(5-Bromo-2-fluoro-phenyl)-[1,2,4]oxadiazol-3-ylmethyl]-piperazine-1-carboxylic acid ethyl ester,
4-[5-(2,5-Dichloro-phenyl)-[1,2,4]oxadiazol-3-ylmethyl]-piperazine-1-carboxylic acid ethyl ester,
4-(5-Thiophen-3-yl-isoxazol-3-ylmethyl)-piperazine-1-carboxylic acid ethyl ester,
4-[5-(2-Fluoro-5-methyl-phenyl)-isoxazol-3-ylmethyl]-piperazine-1-carboxylic acid ethyl ester,
4-{1-[5-(3-Chloro-phenyl)-isoxazol-3-yl]-ethyl}-piperazine-1-carboxylic acid ethyl ester, 4-{1-[5-(2-Fluoro-5-methyl-phenyl)-isoxazol-3-yl]-ethyl}-piperazine-1-carboxylic acid ethyl ester,
(R)- and (S)-4-{1-[5-(2-Fluoro-5-methyl-phenyl)-isoxazol-3-yl]-ethyl}-piperazine-1-carboxylic acid ethyl ester enantiomers,
4-{1-[5-(2-Fluoro-5-methyl-phenyl)-isoxazol-3-yl]-propyl}-piperazine-1-carboxylic acid ethyl ester,
4-{Cyclopropyl-[5-(2-fluoro-5-methyl-phenyl)-isoxazol-3-yl]-methyl}-piperazine-1-carboxylic acid ethyl ester,
4-{1-[5-(2-Fluoro-5-methyl-phenyl)-isoxazol-3-yl]-ethyl}-3-(R)-methyl-piperazine-1-carboxylic acid ethyl ester, (2 diastereomers)
4-{1-[5-(2-Fluoro-5-methyl-phenyl)-isoxazol-3-yl]-ethyl}-3-(S)-methyl-piperazine-1-carboxylic acid ethyl ester, (2 diastereomers)
4-{1-[5-(3-Chloro-phenyl)-isoxazol-3-yl]-ethyl}-3-(R)-methyl-piperazine-1-carboxylic acid ethyl ester, (2 diastereomers)
4-{1-[5-(3-Chloro-phenyl)-isoxazol-3-yl]-ethyl}-3-(S)-methyl-piperazine-1-carboxylic acid ethyl ester, (2 diastereomers)
4-{1-[5-(3-Chloro-phenyl)-isoxazol-3-yl]-ethyl}-2-(R)-methyl-piperazine-1-carboxylic acid ethyl ester, (2 diastereomers)
4-{1-[5-(3-Chloro-phenyl)-isoxazol-3-yl]-ethyl}-2-(S)-methyl-piperazine-1-carboxylic acid ethyl ester, (2 diastereomers)
(R)-4-[5-(3-Chloro-phenyl)-isoxazol-3-ylmethyl]-3-methyl-piperazine-1-carboxylic acid ethyl ester,
(R)-4-[5-(2-Fluoro-5-methyl-phenyl)-isoxazol-3-ylmethyl]-3-methyl-piperazine-1-carboxylic acid ethyl ester,
(S)-4-[5-(3-Chloro-phenyl)-isoxazol-3-ylmethyl]-3-methyl-piperazine-1-carboxylic acid ethyl ester,
(S)-4-[5-(2-Fluoro-5-methyl-phenyl)-isoxazol-3-ylmethyl]-3-methyl-piperazine-1-carboxylic acid ethyl ester,
4-[5-(3-Chloro-phenyl)-oxazol-2-ylmethyl]-piperazine-1-carboxylic acid ethyl ester,
4-[5-(5-Chloro-2-fluoro-phenyl)-[1,2,4]oxadiazol-3-ylmethyl]-piperazine-1-carboxylic acid ethyl ester,
4-[5-(2-Chloro-5-methyl-phenyl)-[1,2,4]oxadiazol-3-ylmethyl]-piperazine-1-carboxylic acid ethyl ester,
4-{1-[5-(3-Chloro-phenyl)-[1,2,4]oxadiazol-3-yl]-ethyl}-piperazine-1-carboxylic acid ethyl ester,
4-{1-[5-(3-Chloro-phenyl)-[1,2,4]oxadiazol-3-yl]-ethyl}-3-(S)-methyl-piperazine-1-carboxylic acid ethyl ester,
4-{1-[5-(3-Chloro-phenyl)-[1,2,4]oxadiazol-3-yl]-ethyl}-3-(R)-methyl-piperazine-1-carboxylic acid ethyl ester,
4-{1-[5-(3-Chloro-phenyl)-[1,2,4]oxadiazol-3-yl]-ethyl}-3-(R)-methyl-piperazine-1-carboxylic acid ethyl ester,
4-[5-(5-Chloro-2-fluoro-phenyl)-[1,3,4]oxadiazol-2-ylmethyl]-piperazine-1-carboxylic acid ethyl ester,
4-{1-[5-(5-Chloro-2-fluoro-phenyl)-[1,3,4]oxadiazol-2-yl]-ethyl}-piperazine-1-carboxylic acid ethyl ester,
4-[5-(2-Fluoro-5-methyl-phenyl)-[1,3,4]oxadiazol-2-ylmethyl]-piperazine-1-carboxylic acid ethyl ester,
4-{1-[5-(2-Fluoro-5-methyl-phenyl)-[1,3,4]oxadiazol-2-yl]-ethyl}-piperazine-1-carboxylic acid ethyl ester,
4-(5-m-Tolyl-isoxazol-3-ylmethyl)-piperazine-1-carboxylic acid ethyl ester,
4-[5-(3-methoxy-phenyl)-isoxazol-3-ylmethyl]-piperazine-1-carboxylic acid ethyl ester,
4-[5-(3-cyano-phenyl)-isoxazol-3-ylmethyl]-piperazine-1-carboxylic acid ethyl ester,
4-[5-(3-Formyl-phenyl)-isoxazol-3-ylmethyl]-piperazine-1-carboxylic acid ethyl ester,
4-[5-(5-Cyano-2-fluoro-phenyl)-isoxazol-3-ylmethyl]-piperazine-1-carboxylic acid ethyl ester,
4-[5-(5-Chloro-2-fluoro-phenyl)-isoxazol-3-ylmethyl]-piperazine-1-carboxylic acid ethyl ester,
4-{1-[5-(5-Chloro-2-fluoro-phenyl)-isoxazol-3-yl]-ethyl}-piperazine-1-carboxylic acid ethyl ester,
4-[1-(5-m-Tolyl-isoxazol-3-yl)-ethyl]-piperazine-1-carboxylic acid ethyl ester,
4-{1-[5-(3-Methoxy-phenyl)-isoxazol-3-yl]-ethyl}-piperazine-1-carboxylic acid ethyl ester,
4-{1-[5-(3-Cyano-phenyl)-isoxazol-3-yl]-ethyl}-piperazine-1-carboxylic acid ethyl ester,
4-{1-[5-(5-Cyano-2-fluoro-phenyl)-isoxazol-3-yl]-ethyl}-piperazine-1-carboxylic acid ethyl ester,
4-{1-[5-(2-Methyl-pyridin-4-yl)-isoxazol-3-yl]-ethyl}-piperazine-1-carboxylic acid ethyl ester,
4-{1-[5-(5-Chloro-2-fluoro-phenyl)-isoxazol-3-yl]-2,2,2-trifluoro-ethyl}-piperazine-1-carboxylic acid ethyl ester,
4-[5-(2-Fluoro-5-iodo-phenyl)-[1,2,4]oxadiazol-3-ylmethyl]-piperazine-1-carboxylic acid ethyl ester,
4-[5-(2-Hydroxy-5-methyl-phenyl)-[1,2,4]oxadiazol-3-ylmethyl]-piperazine-1-carboxylic acid ethyl ester,
4-[5-(5-Chloro-2-hydroxy-phenyl)-[1,2,4]oxadiazol-3-ylmethyl]-piperazine-1-carboxylic acid ethyl ester,
or salt thereof.

9. A pharmaceutical formulation comprising as active ingredient a therapeutically effective amount of a compound according to any one of claims 1 to 8 in association with one or more pharmaceutically acceptable diluent, excipients and/or inert carrier.

10. The pharmaceutical formulation according to claim 9, for use in the prevention and/or treatment of mGluR5 receptor-mediated disorders.

11. A compound according to any one of claims 1 to 8 for use in therapy.

12. The compound according to claim 11, for use in prevention and/or treatment of mGluR5 receptor-mediated disorders.

13. The use of a compound according to any one of claims 1 to 8 in the manufacture of a medicament for the use in the prevention and/or treatment of mGluR5 receptor-mediated disorders.

14. A method of prevention and/or treatment of mGluR5 receptor-mediated disorders, comprising administering to a mammal, including man in need of such prevention and/or treatment, a therapeutically effective amount of a compound according to any one of claims 1 to 8.

15. The method according to claim 14, for use in prevention and/or treatment of neurological disorders.

16. The method according to claim 14, for use in prevention and/or treatment of psychiatric disorders.

17. The method according to claim 14, for use in prevention and/or treatment of chronic and acute pain disorders.

18. A method for inhibiting activation of mGluR5 receptors, comprising treating a cell containing said receptor with an effective amount of a compound according to any one of claims 1 to 8.

19. Processes for the preparation of a compound according to claim 1, comprising; 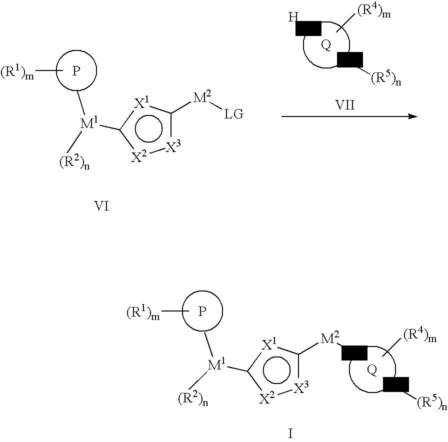
wherein LG is any suitable leaving group such as chloro or mesylate or a group which may subsequently be transformed into a leaving group and P, Q, X¹, X², X³, X⁴, X⁵, R¹, R², R⁴, R⁵, M¹, M², m and n are as defined in claim 1.

20. A compound which is,
N,N-Bis-(2-trifluoromethanesolfonyl-ethyl)-2-nitrobenzenesulfonamide,
(Cyano-methyl-methyl)-carbamic acid tert-butyl ester,
2-Chloro-N-hydroxy-acetamidine,
[1-(N-Hydroxycarbamimidoyl)-ethyl]-1-carbamic acid tert-butyl ester,
3-Chloromethyl-5-m-tolyl-[1,2,4]oxadiazole,
3-(3-Chloromethyl-[1,2,4]oxadiazol-5-yl)-benzonitrile,
3-Chloromethyl-5-(3-fluoro-phenyl)-[1,2,4]oxadiazole,
3-Chloromethyl-5-(3-iodo-phenyl)-[1,2,4]oxadiazole,
3-Chloromethyl-5-(3-chloro-phenyl)-[1,2,4]oxadiazole,
3-Chloromethyl-5-(3-trifluoromethoxy-phenyl)-[1,2,4]oxadiazole,
5-(3-Bromo-phenyl)-3-chloromethyl-[1,2,4]oxadiazole,
1-(5-(3-Methylphenyl-[1,2,4]oxadiazol-3-yl)-ethylamine,
1-[1-(5-(3-Methyl-phenyl)-[1,2,4]oxadiazol-3-yl)-ethyl]-piperazine,
1-(5-m-Tolyl-[1,2,4]oxadiazol-3-ylmethyl)-piperazine or
1-[5-(3-Methoxy-phenyl)-[1,2,4]oxadiazol-3-ylmethyl]-3-methyl-piperazine for use as an intermediate in the preparation of a compound according to claim 1.
